(19) United States
(12) Patent Application Publication
(10) Pub. No.: US 2014/0234357 A1
(43) Pub. Date: Aug. 21, 2014

(54) H5 PROTEINS OF H5N1 INFLUENZA VIRUS FOR USE AS A MEDICAMENT

(71) Applicant: Egbert Siegfried MUNDT, Isernhagen (DE)

(72) Inventor: Egbert Siegfried MUNDT, Isernhagen (DE)

(73) Assignee: BOEHRINGER INGELHEIM VETMEDICA GMBH, Ingelheim am Rhein (DE)

(21) Appl. No.: 14/184,919

(22) Filed: Feb. 20, 2014

(30) Foreign Application Priority Data
Feb. 21, 2013 (EP) PCT/EP2013/053505

(51) Int. Cl. A61K 39/145; C07K 14/005

(52) U.S. Cl. CPC A61K 39/145; C07K 14/005
USPC 424/186.1; 530/350

(57) ABSTRACT

The present invention is based on the surprising finding that H5 protein of clade 1 H5N1 induces, in particular by a single-shot vaccination, a cross-clade protective immune response to influenza viruses with H5N1 HA. In one aspect, the invention is thus directed to H5 protein of clade 1 H5N1 virus for use in a method of treating or preventing infections with H5N1 virus of a different clade, wherein the H5N1 virus of a different clade comprises a particular polynucleotide and/or H5 protein.

### SEQUENCE LISTING

This application contains a sequence listing in accordance with 37 C.F.R. 1.821-1.825. The sequence listing accompanying this application is hereby incorporated by reference in its entirety.

### FIELD OF THE INVENTION

The present invention relates to the field of medicine, preferably to the field of infectious diseases. In particular the present invention relates to influenza proteins and vaccines. Most particularly, the present invention relates to the use of any of such proteins or vaccines for the treatment and prevention of influenza infections, furthermore for the prevention of intra- and inter-species transmission of influenza virus.

### BACKGROUND OF THE INVENTION

Influenza infection remains an important infection in animals and humans. Influenza is caused by viruses that undergo continuous antigenic changes/modifications and that possess an animal reservoir. Thus new epidemics and pandemics may occur in the future, and eradication of the disease will be difficult to achieve. Influenza viruses are well known in the art and described more in detail for example by P. Palese, Nature Medicine, vol. 10, no. 12, pp. S 82 to S 86 of December 2004, with further references. Briefly, the genome of the influenza A virus consists of eight single-stranded segments and the viral particles has two major glycoproteins on its surface: hemagglutinin (H) and neuraminidase (N). With at least 16 different hemagglutinin (H1 to H16) and 9 different neuraminidase (N1 to N9) subtypes, there is a considerable antigenic variation among influenza viruses.

Influenza virus of type H5N1 Fowl Plague virus has been demonstrated to infect poultry, pigs and man. The viruses can also be transmitted directly from avian species to humans (Claas et al., Lancet 1998, 351: 472; Suarez et al., J. Virol. 1998, 72: 6678; Subbarao et al., Science 1998, 279: 393; Shortridge, Vaccine 1999, 17 (Suppl. 1): S26-S29). Mortality in known human clinical cases approaches about 50%.

Over the last century pigs have been an important vector for influenza pandemics. Pigs, camels, and seals, preferably pigs, can serve as a ‘mixing chamber’ for avian influenza viruses, and therefore represent a potential risk factor for overcoming the species hurdles from poultry, the naturally reservoir of influenza viruses, to mammals. This normally occurs by double infections of the susceptible animals, e.g. pig, with both, an established mammalian (porcine), as well as an avian influenza virus. This double infection may create new recombinant viruses that may be the cause of human or porcine pandemics. Recent evidence would, however, indicate that a recombination of current avian H5 strains with mammalian influenza viruses will not result in highly virulent recombinants. On the other hand, avian influenza virus can infect pigs and by spontaneous mutations can become adapted to pigs. The critical hurdle will be overcome as soon as the virus can cause horizontal infections within a pig (or other mammalian) population.

Yet, a major part of Southeast Asian pigs have been infected with avian (H5) influenza virus strains originating from neighboring poultry husbandry. As those infections have so far been sub-clinical, they can only be diagnosed by laboratory methods and thus are frequently overlooked. There is a high risk that those sub-clinically-infected pigs will serve as an opportunity for the virus to adapt to the mammalian system, spread within the porcine population, and also infect human beings.

Current influenza vaccines include a subunit vaccine (Babai et al., Vaccine 1999, 17(9-10):1223-1238; Crawford et al., Vaccine 1999, 17(18):2265-2274; Johansson et al., Vaccine 1999, 17(15-16):2073-2080) attenuated vaccine (Horimoto et al., Vaccine 2004, 22(17-18):2244-2247), DNA vaccine (Watabe et al., Vaccine 2001, 19(31):4434-4444) and inactivated influenza vaccine (Cao et al., Vaccine 1992, 10(4):238-242), with the latter being the most widely used on a commercial scale (Lipatov et al., J Virol 2004, 78(17):8951-8959).

Subunit vaccines, recombinant hemagglutinin and neuraminidase (Babai et al., Vaccine 1999, 17(9-10):1223-1238; Crawford et al., Vaccine 1999, 17(18):2265-2274; Johansson et al., Vaccine 1999, 17(15-16):2073-2080) may be an attractive alternative to the inactivated vaccine, although none are currently in use as commercial vaccines. The preparation of such vaccines is obviously safer than for an inactivated vaccine. Moreover, subunit vaccines do not generate antibody responses to internal influenza viral proteins and thus allow distinction between vaccinated and infected animals (Crawford et al., Vaccine 1999, 17(18):2265-2274).

Hemagglutinin protein is the receptor-binding and membrane fusion glycoprotein of influenza virus and the target for infectivity-neutralizing antibodies. The entire hemagglutinin protein (HA) from the H5N1 is composed of 568 amino acids, with a molecular weight of 56 kDa. The HA molecule consists of HA1 and HA2 subunits, with the HA1 subunit mediating initial contact with the cell membrane and HA2 being responsible for membrane fusion (Chizmadzhev, Bioelectrochemistry 2004, 63(1-2):129-136).

Baculovirus/insect cell systems have been used to express hemagglutinin genes isolated from avian influenza subtypes (Babai et al., Vaccine 1999, 17(9-10):1223-1238; Crawford et al., Vaccine 1999, 17(18):2265-2274; Johansson et al., Vaccine 1999, 17(15-16):2073-2080); Nwe et al., BMC Microbiology 2006, 6(16):doi:10.1186/1471-2180-6-16). However, those recombinant proteins seem not to be protective in any case, or only less effective at least for some species (Treanor et al., Vaccine 2001, 19: 1732-1737).

The document Lin et al. (J Vet Med. Sci. 2008 70(11):1147-52) discloses the use of a baculovirus/insect cell system for the production of H5 protein of clade 2 H5N1 virus A/duck/China/E319-2/03, which is usable for a prime-booster vaccination for preventing an infection with the clade 2 virus A/duck/China/E319-2/03.

Bright et al. (PLoS One. 2008 30;3(1):e1501) describes the use of a baculovirus/insect cell system for generating virus-like particles (VLPs) which include neuraminidase, hemagglutinin and matrix 1 protein from clade 2 H5N1 virus for inducing a cross-clade protective immune response against a challenge with clade 1 H5N1 virus A/VN/1203/2004 in mice. However, the production of VLPs is not without problems, since in order to generate a functional VLP that effectively mimic a real virus, multiple virus structural proteins are needed which must then be correctly assembled into a particle that reproduces the confirmation of the outer shell (capsid) of the infectious virus. Further, study also reals that in vitro assembly of VLPs competes with aggregation (Ding et al. Biotechnology and Bioengineering 107 (3): 550-560).

Thus, there is a need to increase availability of improved vaccines and new vaccination approaches to provide better approaches to control influenza infections and to have a positive impact on disease load. In particular, there is a strong need for a simple, effective and easy-to-handle system inducing, preferably by a single-shot vaccination, a cross-clade protective immune response to influenza viruses with H5N1 HA.

### DESCRIPTION OF THE INVENTION

Before the embodiments of the present invention it shall be noted that as used herein and in the appended claims, the singular forms “a”, “an”, and “the” include plural reference unless the context clearly dictates otherwise. Thus, for example, reference to “a preparation” includes a plurality of such preparations; reference to the “carrier” is a reference to one or more carriers and equivalents thereof known to those skilled in the art, and so forth. Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was omitted from the description. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

The solution to the above technical problem is achieved by the description and the embodiments characterized in the claims.

### Influenza Proteins and Nucleic Acid Molecules Coding for Those

The present invention is based on the surprising finding that H5 protein of clade 1 H5N1 induces, in particular by a single-shot vaccination, a cross-clade protective immune response to influenza viruses with H5N1 HA. As one feature, the H5 protein of clade 1 H5N1 virus, which is for reasons of clarity also termed “H5 protein (1)” herein, comprises or consists of a polypeptide sequence having at least 98% sequence identity with the polypeptide sequence set forth in SEQ ID NO:1.

Sequence identity with a sequence, as mentioned herein, in particular means sequence identity over the length of said sequence or sequence identity over the whole length of said sequence, respectively.

A “single-shot vaccination” refers to an immunogenic composition that is effective at reducing the incidence of or severity of infection after a single dose thereof, without the need for a booster.

In one aspect, the invention is thus directed to H5 protein (1) of clade 1 H5N1 virus for use in a method of treating or preventing infections with H5N1 virus of a different clade, namely of a clade different from clade 1 or from any clade with the exception of clade 1, respectively, wherein said H5 protein (1) comprises or consists of a polypeptide sequence having at least 98% sequence identity with the polypeptide sequence of SEQ ID NO:1.

The term “clade” or “clades” as used herein relates to the clade(s) of the WHO Nomenclature System for the highly Pathogenic Avian Influenza Virus (H5N1), which is summarized at the WHO website URL:

who.int/csr/disease/avian_influenza/guidelines/nomenclature/en/ (12 Aug. 2011), which is incorporated herein by reference
10 distinct initial clades of viruses (numbered 0-9) are defined (WHO/OIE/FAO H5N1 Evolution Working Group, 2008), which are called first order clades. Clades are strictly defined on the nucleotide level as meeting the following three specific clade definition criteria developed by the WHO/OIE/FAO H5N1 Evolution Working Group:
sharing of a common (clade-defining) node;
monophyletic grouping with a bootstrap value of ≧60 at the clade-defining node (after 1000 neighbor-joining bootstrap replicates); and
average percentage pairwise nucleotide distances between and within clades of >1.5% and <1.5%, respectively.

As the viruses within these 10 clades continue to evolve, new sublineages (potential H5N1 clades) periodically emerge. Once these sublineages meet the same three specific clade definition criteria as the initial 10 clades (numbered 0-9), they are designated as separate clades (WHO/OIE/FAO H5N1 Evolution Working Group Emerg. Inf. Dis. 14, 7 (2008). These new clades are defined as second (or third, etc.) order clades and assigned a numerical ‘address’ which links them to their original clade using a hierarchical decimal numbering system. For example, within the antigenically distinct clade 2.3, third order clades meeting the clade definition are designated as clades 2.3.1 and 2.3.2 and so on. This logical hierarchal numbering system is objectively related to HA phylogeny.

The criteria used for the clade designation according to the WHO Nomenclature System for H5N1 are:
1 Maintain previously designated clade numbers where possible (i.e., clade 2.2 remains 2.2 and clade 1 remains 1)
2 New clade designations based on phylogenetic tree topology derived from all available sequences (the large tree)
   H5N1 progenitors (closest to Gs/Guangdong/1/96) re-designated as clade 0 Subsequent clades numbered starting from clade 3 (i.e., clades 3-9)
   Clades designated by presence of a distinct common node shared by at least 4 isolates (in a monophyletic group)
   Additional branches designated as a single clade evolves into more than one distinct lineage (i.e., clade 2.2 or clade 2.3.1; based on sharing of a common node and monophyletic grouping)
3 Average percentage pairwise distances between and within clades (using Kimura 2-parameter)
   Distinct clades should have >1.5% average distances between other clades
   Distinct clades should have <1.5% average distances within the clade (may be slightly higher in clades with highly evolved outliers; i.e., Ck/Shanxi/2/2006 in clade 7)
4 Bootstrap (based on 1,000 neighbor-joining bootstrap replicates)>60% bootstrap value at clade-defining node
   (taken from Table 1 of: WHO/OIE/FAO H5N1 Evolution Working Group Emerg. Inf. Dis. 14, 7 (2008)).

The prototype strain for each clade is listed in the following Table:

| Clade | Prototype strain |
|---|---|
| 0 | Gs/Guangdong/1/96 |
| 3 | Ck/Hong Kong/YU562/2001 |
| 4 | Gs/Guiyang/337/2006 |
| 5 | Gs/Guangxi/914/2004 |
| 6 | Ck/Hunan/01/2004 |
| 7 | Ck/Shanxi/2/2006 |
| 8 | Ck/Hong Kong/YU777/2002 |
| 9 | Dk/Guangxi/2775/2005 |
| 1 | Vietnam/1203/2004 |
| 2.1.1 | Ck/Indonesia/BL/2003 |
| 2.1.2 | Indonesia/538H/2006 |
| 2.1.3 | Indonesia/5/2005 |
| 2.2 | BHGs/Qinghai/1A/2005 |
| 2.3.1 | Dk/Hunan/303/2004 |
| 2.3.2 | Ck/Guangxi/2461/2004 |
| 2.3.3 | Ck/Guiyang/3055/2005 |
| 2.3.4 | Dk/Fujian/1734/2005 |
| 2.4 | Ck/Yunnan/115/2004 |
| 2.5 | Ck/Korea/ES/2003 |
| 2.5 | Ck/Korea/ES/2003 |

(taken from Table 2 of: WHO/OIE/FAO H5N1 Evolution Working Group Emerg. Inf. Dis. 14, 7 (2008)).

The publication WHO/OIE/FAO H5N1 Evolution Working Group Emerg. Inf. Dis. 14, 7 (2008), which is incorporated herein by reference, is found at the CDC website URL: cdc.gov/EID/content/14/7/el.htm (12 Aug. 2011).

An overview of the clade classification of known H5N1 viruses is provided by the phylogenetic tree at the WHO website URL: who.int/csr/disease/avian_influenza/H5CompleteTree.pdf (15 Aug. 2011), which is hereby incorporated by reference.

For determining the clade of a H5 protein of H5N1, for example, the web based tool “Highly Pathogenic Avian Influenza (HPAI) H5N1 HA clade prediction” can be used, which is described by Lu, Davis, Rowley, and Donis: “A Web-based tool for the clade designation of highly pathogenic avian influenza H5N1 viruses” in Options for the Control of Influenza VI. J. M. Katz, N. Cox & A. W. Hampson (Eds.) London: Blackwell, 2007, herein incorporated by reference, and which is found at the website URL: h5n1.flugenome.org/grouping.php (12 Aug. 2011).

For example, a H5 protein of clade 1 H5N1 virus (H5 protein (1)) is thus a HA with an amino acid sequence encoded by a nucleotide sequence of a clade 1 according to the above-mentioned WHO Nomenclature System for H5N1.

A clade 2.3.1 H5N1 virus, for instance, is hence a H5N1 falling under the criteria of a clade 2.3.1 according to the above-mentioned WHO Nomenclature System for H5N1.

In a preferred embodiment, the H5 protein (1) according to the invention, namely the H5 protein of clade 1 H5N1 virus as described herein, comprises or consists of a polypeptide sequence having at least 98.1%, preferably at least 98.2%, more preferably at least 98.3%, and most preferably at least 98.4% sequence identity with the polypeptide sequence of SEQ ID NO:1.

Sequence identity in the context of the invention is understood as being based on determined pairwise similarity between protein sequences. The determination of percent similarity between two sequences is preferably accomplished using a computational algorithm, in particular the well-known Basic Local Alignment Search Tool (Altschul S F, Gish W, Miller W, Myers E W, Lipman D J: Basic local alignment search tool. J Mol Biol 1990, 215(3):403-410). For purposes of the present invention, percent sequence identity of an amino acid sequence is determined using the BLAST blastp homology search algorithm using the following parameters: an expected threshold of 10, word size 3, BLOSUM62 matrix, gap opening penalty of 11, a gap extension penalty of 1, and conditional compositional score matrix adjustment. The database to search against is the set of non-redundant protein sequences (nr). The BLAST homology search algorithm is described in Altschul S F (1990), J Mol Biol 1990, 215(3):403-410, which is herein incorporated by reference.

A variant may, for example, differ from the reference accession number BAE07201 molecule without signal peptide (N-terminal 16 amino acid residues are not shown in SEQ ID NO:1) by as few as 1 to 15 amino acid residues, as few as 1 to 10 amino acid residues, such as 6-10, as few as 5, as few as 4, 3, 2, or even 1 amino acid residue.

In one exemplary embodiment, the H5 protein (1) according to the invention, i.e. the H5 protein (1) of clade 1 H5N1 virus for use in a method of treating or preventing infections with H5N1 virus of a different clade, is preferably a H5 protein of influenza virus, wherein the H5 protein having the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted. Said preferred H5 protein (1) is also termed Mut k+ or mutK+ in the following. Preferably, such H5 protein and any further H5 protein according to the invention is an isolated H5 protein.

The term “H5 protein (1) of clade 1 H5N1”, as used herein, preferably means “H5 protein (1) as single antigen of clade 1 H5N1 virus” or in particular “H5 protein (1) as single antigen”.

The terms “hemagglutinin 5 (H5)” or “H5 of avian influenza virus” or “H5 protein” as used herein are equivalent and mean, but are not limited to any naturally occurring H5 protein and any modified forms of H5 protein, including any deletion, substitution and/or insertion mutant of H5 protein.

The numbering of the amino acid positions of the H5 protein (1) Mut k+ as used herein refers to the amino acid position as exemplarily given in SEQ ID NO:2. SEQ ID NO:2 represents the amino sequence of the hemagglutinin of strain duck/China/E319-2/03 but lacking the amino terminal signal peptide. In other words, if reference is made to the amino acid at position 223 (amino acid 223), the amino acid residue is meant which corresponds to amino acid 223 of SEQ ID NO:2. However, this does not mean that the H5 protein Mut k+ according to the invention has the identical amino acid sequence with SEQ ID NO:2. It only says, that the corresponding amino acids of the H5 proteins according to the inventions code for the amino acid residue, as explicitly mentioned. In the current case, amino acid 223 would be Serine (S). The terms “223N”, or “155N” exemplarily mean, that the amino acid at positions 223 and 155, respectively—numbering according to the amino acid positions of SEQ ID NO:2—that amino acid shall code for the amino acid Asparagine (N). In other words, if reference is made to “H5 protein (1) having the amino acid 223N”, a H5 amino acid molecule that normally codes for Serine at amino acid position 223—numbering according to the amino acid positions of SEQ ID NO:2—that amino acid shall be substituted by an Asparagine (N). The term “328K+” or “modification 328K+” means, that at amino acid position 328 of H5 protein—numbering according to the amino acid positions of SEQ ID NO:2—, a second Lysine (K+) is inserted. In cases were amino acids sequences at positions 328 and 329 naturally codes for Lysine-Lysine, no further Lysine (K) shall be inserted. However, most of the known H5 sequences code at amino acid positions 328 and 329 for Lysine-Arginine. In any such cases, the term 328K+ modification means that a second Lysine (K) shall be inserted between Lysine at position 328 and Arginine at position 329. The modified sequence would read then Lysine-Lysine-Arginine (KKR).

Regarding the present example, the hemagglutinin of strain duck/China/E319-2/03 is shifted to a H5 protein (1) of clade 1 H5N1, since it resembles the H5 sequence of the clade 1 H5N1 virus A/HongKong/213/2003, the year/location/host of this HK isolate, and shows reactivity with clade-1-specific antibodies. Hence the Mut K+ sequence is classified as a H5 sequence of a clade 1 H5N1. Within the context of the invention, the designed Mut K+ sequence is thus understood and defined to be a H5 protein of clade 1 H5N1 virus.

Thus, in particular also any designed H5 protein is understood and defined as a H5 protein of clade 1 H5N1 virus according to the invention, if it is encoded by a nucleotide sequence which fulfils the criteria of a nucleotide sequence of a clade 1 according to the above-mentioned WHO Nomenclature System for H5N1.

Thus, in one embodiment, the present invention is implemented with a H5 protein and any modified forms of H5 protein, including any deletion, substitution and/or insertion mutant of H5 protein, wherein those H5 proteins having the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted. It is self-explanatory, that any of the H5 proteins as provided herewith are antigenic, which mean they show antigenic properties in a standard hemagglutinin inhibition assay for influenza viruses.

According to a further embodiment, the present invention also relates to any part of the H5 protein (1), which means any peptide-fragment which shows antigenic properties in an standard hemagglutinin inhibition assay, having in one embodiment at least the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted.

A H5 protein (1) shows antigenic properties if it inhibits hemagglutination in a standard hemagglutinin inhibition assay, for example, as described in Example 2. Normally said antigenic part of H5 protein (1) comprises 200, 180, 160, 150, 140, 130, 120, 110 or most preferably 105 contiguous amino acids of the amino acid sequence that codes for the H5 protein as mentioned above, modified or non-modified, which shows antigenic properties in a standard hemagglutinin inhibition assay as described in Example 2. A standard hemagglutinin inhibition assay for example is also described in Stephenson et al., Virus Research vol. 103, pp. 91-95 (2004) with further references. However, the HI assay as described in Example 2 shall be understood to be the relevant reference assay in connection with all aspects of the invention as described herein:

Briefly, HI assay was performed to detect the presence of HA-specific antibodies. A heterologous H5N2 virus, A/chicken/Mexico/232/94, was used at a concentration of four hemagglutinating units [4 HA units] in the HI assay. In U-bottomed microtiter plates serial two-fold serum dilutions in PBS were subsequently mixed with equal volumes (25 μL) containing 4 HA units of virus, and incubated at room temperature (about 25° C.) for 30 min Chicken red blood cells, at a concentration of 0.5% in PBS, were added to the serum-virus containing wells and incubated for 40 min at room temperature. The HI titers were determined as reciprocals of the highest serum dilutions in which inhibition of hemagglutination was observed.

Of note, Haesebrouck and Pensaert (1986) found “that there may exist a correlation between the HI titers against the challenge virus and protection from challenge”. Haesebrouck and Pensaert (1986) also determined that pigs with HI titers of ≧40 were “completely resistant to challenge and no replication of the virus occurred in the respiratory tract at challenge”. Thus, the development of HI titers ≧40 in the vaccinated swine would correlate to protection. (F. Haesebrouck and M. B. Pensaert, 1986). Effect of intratracheal challenge of fattening pigs previously immunized with an inactivated influenza H1N1 vaccine (Veterinary Microbiology, 11 (1986) 239-249. It has to assume that equivalent or at least nearly equivalent H5 HI titers will also result in a complete immune protection of swine against avian influenza virus. Lower titers, at least result in a seroconversion of the vaccinated animals and result in partial immune protection of those animals, which also can dramatically reduce the risk of a pandemic.

Moreover, an antigenic part of the H5 protein (1) according to the invention includes, but is not limited to deletion mutants of H5 protein, which comprises:
i. at least 35, 30, 25, 20, 18, 15, 13, 10, 9, or most preferably 8 contiguous amino acids of the amino acid sequence that surrounds and includes the amino acid 223N; and

ii. at least 35, 30, 25, 20, 18, 15, 13, 10, 9, or most preferably 8 contiguous amino acids of the amino acid sequence that surrounds and includes the amino acid modification 328K+, and
iii. wherein any of such antigenic part of H5 protein shows hemagglutinin inhibition in a standard hemagglutinin inhibition assay as described in Example 2.

Preferably, those surrounding amino acids of amino acid 223N and/or 328K+ are encoded by SEQ ID NO:2 or SEQ ID NO:5.

Furthermore preferred H5 proteins (1) according to the invention are:
i. any of those mentioned above having the amino acid 223N and the modification 328K+;
ii. any of those mentioned above having the amino acid 94N/223N and the modification 328K+;
iii. any H5 protein of avian origin having the amino acid 223N, and the modification 328K+, wherein avian origin means that the H5 sequence derived form a virus isolate that was originally isolated from a poultry infected with avian influenza virus type 5; or
iv. any H5 protein of avian origin having the amino acids 94N/223N and the modification 328K+, wherein avian origin means that the H5 sequence derived from a virus isolate that was originally isolated from poultry infected with avian influenza virus type 5; or
v. any H5 protein of avian origin having the amino acids 155N/223N and the modification 328K+, wherein avian origin means that the H5 sequence derived from a virus isolate that was originally isolated from poultry infected with avian influenza virus type 5; or
vi. any H5 protein of avian origin having the amino acid 120N/155N/223N and the modification 328K+, wherein avian origin means that the H5 sequence derived from a virus isolate that was originally isolated from poultry infected with avian influenza virus type 5; or
vii. any H5 protein having the modifications 94N/223N and the modification 328K+; or
viii. any H5 protein having the modifications 94N/155N/223N and the modification 328K+; or;
ix. any H5 protein having the modifications 94N/120N/155N/223N and the modification 328K+; or
x. any H5 protein having the modifications 223N, the modification 328K+, and one or more of the following amino acid clusters selected from the group consisting of:
   a. aa 93-95: GNF
   b. aa 123-125: SDH
   c. aa 128-130: SSG
   d. aa 138-140: GSS
   e. aa 226-228: MDF
   f. aa 270-272: EVE
   g. aa 309-311: NKL; or
xi. any H5 protein having the amino acid 223N, and the modification 328K+, and one or more of the following amino acid clusters selected from the group consisting of:
   a. aa 93-95: GNF
   b. aa 128-130: SSG
   c. aa 138-140: GSS; or
xii. any H5 protein having the amino acid sequence of SEQ ID NO:5.

Furthermore preferred H5 proteins (1) as provided herewith include the H5 proteins as described by Hoffmann et al, PNAS, vol. 106, no. 36, pp. 12915-12920 of Sep. 6, 2005, wherein that H5 proteins includes one or more of the modifications as described above, at least the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted. The disclosure of this reference shall be entirely included herein by reference.

Furthermore preferred H5 proteins (1) as provided herewith include H5 proteins which comprise a peptide that comprises the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted, and:
i. the amino acid sequences of SEQ ID NO:2, SEQ ID NO:3, SEQ ID NO:4; SEQ ID NO:5; SEQ ID NO:6 or SEQ ID NO:7 or;
ii. any peptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of i) that comprises hemagglutinin inhibition in a standard hemagglutinin inhibition as described above; or
iii. any antigenic part of the polypeptides of i) or ii) comprising at least 35, 30, 25, 20, 18, 15, 13, 10, 9, or most preferably 8 contiguous amino acids of any of peptides of i) or ii).
iv. any peptides of i), ii) or iii) having the amino acids 36T, 36K, 83A, 83T, 83D, 86A, 86V, 120N, 120S, 155N, 155S, 156A, 156T, 189R, 189K, 212K, 212R, 212E, 223N, 223N, or 120N/155N.
v. any peptide of i), ii), iii) or iv) having one or more of the following amino acid clusters selected from the group consisting of:
   a. aa 93-95: GNF
   b. aa 123-125: SDH
   c. aa 128-130: SSG
   d. aa 138-140: GSS
   e. aa 226-228: MDF
   f. aa 270-272: EVE
   g. aa 309-311: NKL; or
vi. any peptide of i), ii) iii) or iv) having one or more of the following amino acid clusters selected from the group consisting of:
   a. aa 93-95: GNF
   b. aa 128-130: SSG
   c. aa 138-140: GSS.

“Sequence homology”, as used herein, refers to a method of determining the relatedness of two sequences. To determine sequence homology, two or more sequences are optimally aligned, and gaps are introduced if necessary. In contrast to sequence identity, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homolog sequence comprises at least a stretch of 50, even more preferred of 100, even more preferred of 250, even more preferred of 500 nucleotides. Upon such alignment, sequence homology is ascertained on a position-by-position basis, e.g., the sequences are “homolog” at a particular position if at that position, the nucleotides or amino acid residues are identical. The total number of such position identities is then divided by the total number of nucleotides or amino acid residues in the reference sequence to give % sequence homology. Sequence homology can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence homology are designed to give the largest match between the sequences tested. Methods to determine sequence homology are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence homology between the given and reference sequences.

Furthermore preferred H5 proteins (1) include H5 proteins which comprise the 328K+ modification as mentioned above, and the amino acid sequence provided in TABLE 1, or any immunogenic part thereof:

TABLE 1
H5 antigens

| | Basic- | Amino acid positions^{#} | | | | | | | | | |
|---|---|---|---|---|---|---|---|---|---|---|---|
| Sequence name | sequence | 36 | 83 | 86 | 120 | 155 | 156 | 189 | 212 | 223 | 263 |
| 223N/328K+ | any HA H5 | — | — | — | — | — | — | — | — | N | — |
| 36T/223N/328K+ | any HA H5 | T | — | — | — | — | — | — | — | N | — |
| 36K/223N/328k+ | any HA H5 | K | — | — | — | — | — | — | — | N | — |
| 83A/223N/328k+ | any HA H5 | — | A | — | — | — | — | — | — | N | — |
| 83T/223N/328k+ | any HA H5 | — | T | — | — | — | — | — | — | N | — |
| 83D/223N/328k+ | any HA H5 | — | D | — | — | — | — | — | — | N | — |
| 86A/223N/328k+ | any HA H5 | — | — | A | — | — | — | — | — | N | — |
| 86V/223N/328k+ | any HA H5 | — | — | V | — | — | — | — | — | N | — |
| 120N/223N/328k+ | any HA H5 | — | — | — | N | — | — | — | — | N | — |
| 120S/223N/328k+ | any HA H5 | — | — | — | S | — | — | — | — | N | — |
| 155N/223N/328k+ | any HA H5 | — | — | — | — | N | — | — | — | N | — |
| 155S/223N/328k+ | any HA H5 | — | — | — | — | S | — | — | — | N | — |
| 156A/223N/328k+ | any HA H5 | — | — | — | — | — | A | — | — | N | — |
| 156T/223N/328k+ | any HA H5 | — | — | — | — | — | T | — | — | N | — |
| 189R/223N/328k+ | any HA H5 | — | — | — | — | — | — | R | — | N | — |
| 189K/223N/328k+ | any HA H5 | — | — | — | — | — | — | K | — | N | — |
| 212K/223N/328k+ | any HA H5 | — | — | — | — | — | — | — | K | N | — |
| 212R/223N/328k+ | any HA H5 | — | — | — | — | — | — | — | R | N | — |
| 212E/223N/328k+ | any HA H5 | — | — | — | — | — | — | — | E | N | — |
| 223N/263A/328k+ | any HA H5 | — | — | — | — | — | — | — | — | N | A |
| 223N/263T/328k+ | any HA H5 | — | — | — | — | — | — | — | — | N | T |
| 120N/155N/223N/328k+ | any HA H5 | — | — | — | N | N | — | — | — | N | — |
| A/duck/China/E319- | AAR99628 | T | A | A | S | D | A | R | K | N | A |
| 2/03/328k+ | | | | | | | | | | | |
| A/duck/China/E319- | AAR99628 | T | A | A | S | D | A | R | K | N | A |
| 2/03_223N/328k+ | | | | | | | | | | | |
| A/duck/China/E319- | AAR99628 | T | A | A | N | D | A | R | K | N | A |
| 2/03_120N/223N/328k+ | | | | | | | | | | | |
| A/duck/China/E319- | AAR99628 | T | A | A | S | N | A | R | K | N | A |
| 2/03_155N/223N/328k+ | | | | | | | | | | | |
| A/duck/China/E319- | AAR99628 | T | A | A | S | N | N | R | K | N | A |
| 2/03_120N/155N/223N/ | | | | | | | | | | | |
| 328k+ | | | | | | | | | | | |
| HA/HK/213/03/328k+ | AY518362 | T | A | A | N | N | A | R | K | N | A |
| HA/Vietnam/1203/04 | | K | T | V | S | S | T | K | R | N | T |
| HA/Vietnam/1203/04_223N/ | | K | T | V | S | S | T | K | R | N | T |
| 328k+ | | | | | | | | | | | |
| HA//Vietnam/3046/04_223N/ | | T | A | V | S | S | T | K | R | N | T |
| 328k+ | | | | | | | | | | | |
| HA/Vietnam/3062/04_223N/ | | T | A | V | S | S | T | K | R | N | T |
| 328k+ | | | | | | | | | | | |
| HA/chicken/Vietnam/ | | T | A | V | S | S | T | K | R | N | T |
| 39/04_223N/328k+ | | | | | | | | | | | |
| HA/falcon/HK- | | T | A | A | S | S | A | K | E | N | A |
| D0028/04_223N/328k+ | | | | | | | | | | | |
| HA/duck/Singapore/3/97_223N/ | | T | D | V | S | N | A | K | E | N | A |
| 328k+ | | | | | | | | | | | |
| HA/HK/156/97/328k+ | | T | A | A | S | S | A | K | E | N | T |

^{#}the amino acid positions given in TABLE 1 refer to the positions as exemplarily defined in SEQ ID NO: 2. In other words amino acid 223 of TABLE 1 refers to the amino acid 223 of the sequence of SEQ ID NO: 2.
— means that the amino acids at this positions are variable as compared to the reference sequence.

Furthermore, the present invention also relates to H5 proteins (1) having at least the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted, and comprises:
i. a peptide having the sequences of NCBI Accession No. AAT65209, CAJ32556, ABC47656, CAF21874, CAF21870, AAC58998, AAC58997, AAC58996, AAC58994, AAC58993, AAC58992, AAC58991, AAC58990, AAC58995, AAS45134, AAN17270, AAN17269, AAN17268, AAN17267, AAN17266, AAN17265, AAN17264, AAN17263, AAN17262, AAN17261, AAN17260, AAN17259, AAN17257, AAN17256, AAN17255, AAN17254, AAA43083, AAA43082, AAB19079, BAE48696, BAE48693, BAE48696, BAE48695, BAE48694, BAE48692, BAE48691, BAE48690, BAE48689, BAE48688, BAE48687, BAE48686, BAE48685, BAE48684, BAE48683, AAC58999, ABC72082, AAV91149, AAP71993, AAP71992, AAP71991, AAP71990, AAP71989, AAP72011, AAP72010, AAP72009, AAP72008, AAP72007, AAP72006, AAP72005, AAP72004, AAP72003, AAP72002, AAP72001, AAP72000, AAP71999, AAP71998, AAP71997, AAP71996, AAP71995, AAP71994, AAF99718, ABF58847, AAG38534, AAC32102, AAC32099, AAL75847, AAC32101, AAC32098, AAC32088, AAC32078, AAR99628, AAC32100, AAM49555, AAL75843, AAL75839, AAD13573, AAD13568, AAF04720, AAF04719, AAC34263, AAR16155, AAD13574, AAD13570, AAD13575, AAD13572, AAD13569, AAD13567, AAD13566, AAK57506, AAG01225, AAG01215, AAG01205, AAG01195, or ABD83813 modified in a manner described above, which means that those sequences include the above-mentioned modifications 223N and 328 K+ which are not part of the wild-type sequences; or
ii. any peptide that has at least 85% sequence homology, more preferably at least about 90% sequence homology, still more preferably at least about 95% sequence homology, even more preferably at least about 97% sequence homology, still even more preferably at least about 98% sequence homology, and even more preferably at least about 99% sequence homology to the polypeptide of i) and that show hemagglutinin inhibition in a standard hemagglutinin inhibition as described above;
iii. any of the peptides of i) or ii) having the amino acids 36T, 36K, 83A, 83T, 83D, 86A, 86V, 120N, 120S, 155N, 155S, 156A, 156T, 189R, 189K, 212K, 212R, 212E, 263A, 263T, or 120N/155N; or
iv. any of such peptides of i), ii), or iii) having one or more of the following amino acid clusters selected from the group consisting of:
   a. aa 93-95: GNF
   b. aa 123-125 SDH
   c. aa 128-130: SSG
   d. aa 138-140: GSS
   e. aa 226-228: MDF
   f. aa 270-272: EVE
   g. aa 309-311: NKL; or
v. any peptide of i), ii) iii) or iv) having one or more of the following amino acid clusters selected from the group consisting of:
   a. aa 93-95: GNF
   b. aa 128-130: SSG
   c. aa 138-140: GSS

Preferably, the H5 protein (1) for use in a method of treating or preventing infections with H5N1 virus of a different clade is recombinantly expressed and/or produced by a baculovirus expression system, preferably in cultured insect cells.

The term “H5 protein (1)” as mentioned herein is thus, in particular, equivalent to the term “recombinant H5 protein” used herein.

Regarding the H5N1 virus of a different clade, as mentioned herein, said H5N1 virus of a different clade is preferably selected from the group consisting of clade 0 H5N1 virus, clade 2 H5N1 virus, clade 3 H5N1 virus, clade 4 H5N1 virus, clade 5 H5N1 virus, clade 6 H5N1 virus, clade 7 H5N1 virus, clade 8 H5N1 virus and clade 9 H5N1 virus.

In a further preferred embodiment of the invention, the H5N1 virus of a different clade is a clade 2.2 H5N1 virus or a clade 2.3 H5N1 virus.

In a particular preferred embodiment of the invention, the H5N1 virus of a different clade is a clade 2.2.1 H5N1 virus or a clade 2.3.2 H5N1 virus.

For reasons of clarity, H5 protein of the H5N1 virus of a different clade is termed “H5 protein (2)” hereinafter. Hence, H5 protein (2) as mentioned herein is in particular a H5 protein coded by the genome of a H5N1 of any clade with the exception of clade 1.

In still a further preferred embodiment, the H5N1 virus of a different clade is a H5N1 virus of North African or of Vietnamese origin, wherein said H5N1 virus of North African origin is preferably a H5N1 virus comprising a H5 protein (2) of influenza virus, wherein said H5 protein (2) has
(a) the amino acids 113D, 126H, 145(−), 156R, 160F, 167T, and 181N, wherein the modification 145(−) means that amino acid position 145 of H5 is deleted, or
(b) the amino acids 87P, 145L, 172T, 201E, 206I, 208K, 254T, 341G and 421K, or
(c) the amino acids 145L, 172T, and 254V,
   and wherein the numbering of the amino acid positions of the H5 protein (2) refers to the amino acid position as exemplarily given in SEQ ID NO:8;
   or wherein said H5 protein (2) consists of or comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46.

In yet a further preferred embodiment, the H5N1 virus of a different clade is a H5N1 virus of North African or of Vietnamese origin, wherein said H5N1 virus of North African origin is preferably a H5N1 virus comprising a polynucleotide encoding a H5 protein (2) of influenza virus, wherein said H5 protein (2) has
(a) the amino acids 113D, 126H, 145(−), 156R, 160F, 167T, and 181N, wherein the modification 145(−) means that amino acid position 145 of H5 is deleted, or
(b) the amino acids 87P, 145L, 172T, 201E, 206I, 208K, 254T, 341G and 421K, or
(c) the amino acids 145L, 172T, and 254V,
   and wherein the numbering of the amino acid positions of the H5 protein (2) refers to the amino acid position as exemplarily given in SEQ ID NO:8;
   or wherein said H5 protein (2) consists of or comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46.

In the context of the invention, said H5 protein (2) according to (a) is a Subclade A protein, and said H5 protein according to (b) or (c) is a Subclade B protein.

It is thus understood that such a H5 protein (2) of North African origin could be, for example, a sequence as set forth in SEQ ID NO: 140, which corresponds to SEQ ID NO: 8 without signal peptide (the N-terminal 16 amino acid residues of SEQ ID NO: 8 are not included in SEQ ID NO: 140), and thus starts with the N-terminal amino acid D17, but wherein said sequence has
(a) the amino acids 113D, 126H, 145(−), 156R, 160F, 167T, and 181N, wherein the modification 145(−) means that amino acid position 145 of H5 is deleted, or
(b) the amino acids 87P, 145L, 172T, 201E, 206I, 208K, 254T, 341G and 421K, or
(c) the amino acids 145L, 172T, and 254V,
   wherein the numbering as exemplarily given in SEQ ID NO:8 is used.

As described herein it is understood that the term “H5N1 virus comprising a polynucleotide encoding a H5 protein (2)” is equivalent to “H5N1 virus comprising a polynucleotide which comprises a sequence encoding a H5 protein (2)” or to “H5N1 virus comprising a polynucleotide comprising a sequence which encodes a H5 protein (2)”, respectively.

Within the context of the invention, it is understood that the term “amino acid” in particular refers to an amino acid residue or, respectively, to an amino acid which has been covalently linked via peptide bonds to two further amino acids or, if the amino acid is N- or C-terminally located in the peptide sequence, to one further amino acid.

The H5 protein (2), as described herein, in particular consists of or comprises an amino acid sequence which is at least 95%, preferably 96%, more preferably 97%, still more preferably 98%, yet more preferably 99% or in particular preferably 100% homologous with any one of the sequences as set forth in SEQ ID NOs: 9 to 47. Preferably, the H5 protein (2) consists of or comprises an amino acid sequence which is at least 95%, preferably 96%, more preferably 97%, still more preferably 98%, yet more preferably 99% or in particular preferably 100% homologous with any one of the sequences as set forth in SEQ ID NOs: 15, 20, or 47. In particular, H5 proteins (2) that comprise or consist of the amino acid sequence set forth in SEQ ID NO: 20 are preferred.

In one preferred embodiment, the H5 protein (2), as described herein, has the amino acid 239N. The term “239N” exemplarily means, that the amino acid at position 239—numbering according to the amino acid positions of SEQ ID NO:8—shall code for the amino acid Asparagine (N). In other words, if reference is made to “H5 protein (2) having the amino acid 239N”, a H5 amino acid molecule that normally codes for Serine at amino acid position 239—numbering according to the amino acid positions of SEQ ID NO:8—that amino acid shall be substituted by an Asparagine (N).

In a particular preferred embodiment, the H5 protein (2), as described herein, does not comprise the influenza A H5N1 hemagglutinin signal sequence, wherein the H5 protein (2) influenza A H5N1 hemagglutinin signal sequence preferably comprises or consists of the first 16 N-terminal amino acids (one letter code) MEKIVLLLAIVSLVKS (SEQ ID NO:51) or M1 E2 K3 I4 V5 L6 L7 L8 A9 I10 V11 S12 L13 V14 K15 S16, respectively, wherein the numbering of said first N-terminal 16 amino acid positions refers to the amino acid positions 1 to 16 as exemplarily given in SEQ ID NO 8, namely referring to the sequence (three letter code) Met Glu Lys Ile Val Leu Leu Leu Ala Ile Val Ser Leu Val Lys Ser (SEQ ID NO:51) as set forth in SEQ ID NO:8.

In a further preferred embodiment the H5 protein (2), as described herein, thus consists of or comprises an amino acid sequence which is at least 95%, preferably 96%, more preferably 97%, still more preferably 98%, yet more preferably 99% or in particular preferably 100% homologous with any one of the sequences as set forth in SEQ ID NOs: 9 to 47, wherein the sequences as set forth in SEQ ID NOs: 9 to 47 start with amino acid D17, and wherein the numbering of the first N-terminal amino acid position (D17) refers to the amino acid position 17 as exemplarily given in SEQ ID NO 8.

Table B provides the respective sequences SEQ ID NOs: 62 to 99 that correspond to variants of the sequences as set forth in SEQ ID NOs: 9 to 46, wherein SEQ ID NOs: 62 to 99 start with the respective amino acid D17 of SEQ ID NOs: 9 to 46, as mentioned herein, and Table D provides the respective variant of sequence SEQ ID NO: 47, i.e. SEQ ID NO: 138, wherein the variant sequence starts with amino acid D17, as mentioned herein.

In one example, the H5 protein (2), as described herein, thus preferably consists of or comprises an amino acid sequence which is at least 95%, preferably 96%, more preferably 97%, still more preferably 98%, yet more preferably 99% or in particular preferably 100% homologous with the polypeptide sequence DQICIGYHANNSTEQVDTIMEKNVTVTHAQDILEKTHNGKLCNLDGVKPLILRDCSV AGWLLGNPMCDEFLNVPEWSYIVEKINPTNDLCYPGNFNDYEELKHLLSRINHFEKI QIIPKNSWSDHEASGVSSACPYQGRSSFFRNVVWLTKKNNAYPTIKKSYNNTNQEDL LVLWGIHHPNDAAEQTRLYQNPTTYISVGTSTLNQRLVPKIATRSKVNGQSGRMEFF WTILKSNDAINFESNGNFIAPENAYKIVKKGDSTIMKSELEYGDCNTKCQTPIGAINSS MPFHNIHPLTIGECPKYVKSNRLVLATGLRNSPQGERRRKKRGLFGAIAGFIEGGWQ GMVDGWYGYHHSNEQGSGYAADKESTQKAIDGVTNKVNSIIDKMNTQFEAVGREF NNLERRIENLNKKMEDGFLDVWTYNAELLVLMENERTLDFHDSNVKNLYDKVRLQ LRDNAKELGNGCFEFYHRCDNECMESVRNGTYDYPQYSEEARLKREEISGVKLESIG TYQILSIYSTVASSLALAIMV (SEQ ID NO:52), wherein this sequence corresponds to a variant of SEQ ID NO:15, wherein the variant starts with amino acid D17 of SEQ ID NO: 15, wherein the numbering of the N-terminal amino acid position (D17) refers to the amino acid position 17 as exemplarily given in SEQ ID NO 8.

In another example, the H5 protein (2), as described herein, thus preferably consists of or comprises an amino acid sequence which is at least 95%, preferably 96%, more preferably 97%, still more preferably 98%, yet more preferably 99% or in particular preferably 100% homologous with the polypeptide sequence DQICIGYHANNSTEQVDTIMEKNVTVTHAQDILEKTHNGKLCDLGGVKPLILRDCSV AGWLLGNPMCDEFPNVSEWSYIVEKINPANDLCYPGNFNNYEELKHLLSRINRFEKI QIIPKSSWPDHEASLGVSSAC PYQGGPSFYRNVVWLIKKNDTYPTIKESYHNTNQEDL LVLWGIHHPNNEEEQKRIYKNPTTYVSVGTSTLNQRLVPKIATRSKVNGQSGRVEFF WTILKSNDTINFESNGNFIAPENAYKIVKKGDSTIMKSELEYGNCSTKCQTPIGAINTS MPFHNIHPLTIGECPKYVKSNRLVLATGLRNSPQGEGRRKKRGLFGAIAGFIEGGWQ GMVDGWYGYHHSNEQGSGYAADKESTQKAIDGVTNKVNSIIDKMNTQFEAVGREF NNLEKRIENLNKKMEDGFLDVWTYNAELLVLMENERTLDFHDSNVRNLYDKVRLQ LRDNAKELGNGCFEFYHRCDNECMESVRNGTYDYPQYSEEARLKREEISGVKLESIG TYQILSIYSTVASSLALAIMVAGLFLWMCSNGSLQCRICI, (SEQ ID NO: 53) wherein the sequence corresponds to SEQ ID NO:20 starting with amino acid D17, and wherein the numbering of the first N-terminal amino acid position (D17) refers to the amino acid position 17 as exemplarily given in SEQ ID NO: 8.

It is thus understood, that the term “variants of SEQ ID NOs: 9 to 47” is preferably also directed to the variant sequences that start with amino acid D17 of SEQ ID NOs: 9 to 47, and wherein the variant sequences of SEQ ID NOs 9 to 46 starting with amino acid D17 are given in Table B and correspond to SEQ ID NOs: 62 to 99, and wherein the sequence of SEQ ID NO: 47 starting with amino acid D17 has the sequence DQICIGYHANNSTEQVDTIMEKNVTVTHAQDILEKTHNGKLCDLDGVKPLILRDCSV AGWLLGNPMCDEFPNVSEWSYIVEKINPANDLCYPGNFNNYEELKHLLSRINRFEKI QIIPKSSWPDHEASLGVSSAC PYQGGPSFYRNVVWLIKKNNTYPTIKESYHNTNQEDL LVLWGIHHPNDEEEQTRIYKNPTTYISVGTSTLNQRLVPKIATRSKVNGQSGRVEFFW TILKSNDTINFESNGNFIAPENAYKIVKKGDSTIMKSELEYGNCNTKCQTPIGAINSSM PFHNIHPLTIGECPKYVKSNRLVLATGLRNSPQGEGRRKKRGLFGAIAGFIEGGWQG MVDGWYGYHHSNEQGSGYAADKESTQKAIDGVTNKVNSIIDKMNTQFEAVGREFN NLEKRIENLNKKMEDGFLDVWTYNAELLVLMENERTLDFHDSNVKNLYDKVRLQL RDNAKELGNGCFEFYHRCDNECMESVRNGTYDYPQYSEEARLKREEISGVKLESIGT YQILSIYSTVASSLALAIMVAGLFLWMCSNGSLQCRICI (SEQ ID NO: 54).

In a further preferred embodiment the H5 protein (2), as described herein, comprises a full length C-terminal sequence, wherein the full length C-terminal sequence preferably corresponds to amino acids 527 to 568, wherein the numbering of said amino acid positions refers to the amino acid positions 527 to 568 as exemplarily given in SEQ ID NO 47. The full length C-terminal sequence is thus preferably the sequence IGTYQILSIYSTVASSLALAIMVAGLFLWMCSNGSLQCRICI (SEQ ID NO: 55), wherein in a further preferred embodiment the Phenyl alanine residue (F) is a Serine (S) residue or wherein in another preferred embodiment the sequence IMVA (SEQ ID NO: 56) is substituted by the sequence IMMA (SEQ ID NO: 57). Hence the full length C-terminal sequence is thus preferably the sequence: IGTYQILSIYSTVASSLALAIMVAGLFLWMCSNGSLQCRICI (SEQ ID NO: 58) or the sequence IGTYQILSIYSTVASSLALAIMVAGLSLWMCSNGSLQCRICI (SEQ ID NO: 59) or the sequence IGTYQILSIYSTVASSLALAIMMAGLSLWMCSNGSLQCRICI (SEQ ID NO: 60).

In yet a further preferred embodiment the H5 protein (2), as described herein, thus consists of or comprises an amino acid sequence which is at least 95%, preferably 96%, more preferably 97%, still more preferably 98%, yet more preferably 99% or in particular preferably 100% homologous with any one of the sequences as set forth in SEQ ID NOs: 9 to 47, sequences as set forth in SEQ ID NOs: 54, 100-137 that correspond to variants of SEQ ID NOs: 9 to 47 that start with amino acid D17 and additionally comprise a full length C-terminal sequence, wherein the numbering of the first N-terminal amino acid position (D17) refers to the amino acid position 17 as exemplarily given in SEQ ID NO 8, and wherein the full length C-terminal sequence corresponds to amino acids 527 to 568, wherein the numbering of these amino acid positions refers to the amino acid positions 527 to 568 as exemplarily given in SEQ ID NO 47.

Table C provides the respective sequences SEQ ID NOs 100 to 137, wherein the sequences start with amino acid D17 and comprise a full-length C-terminal sequence, as described herein, and Table D provides the respective sequence SEQ ID NO: 138, wherein the sequence starts with amino acid D17 of SEQ ID NO: 47 and comprises a full-length C-terminal sequence, as described herein.

In one example, the H5 protein (2), as described herein, thus preferably consists of or comprises an amino acid sequence which is at least 95%, preferably 96%, more preferably 97%, still more preferably 98%, yet more preferably 99% or in particular preferably 100% homologous with the polypeptide sequence DQICIGYHANNSTEQVDTIMEKNVTVTHAQDILEKTHNGKLCNLDGVKPLILRDCSV AGWLLGNPMCDEFLNVPEWSYIVEKINPTNDLCYPGNFNDYEELKHLLSRINHFEKI QIIPKNSWSDHEASGVSSACPYQGRSSFFRNVVWLTKKNNAYPTIKKSYNNTNQEDL LVLWGIHHPNDAAEQTRLYQNPTTYISVGTSTLNQRLVPKIATRSKVNGQSGRMEFF WTILKSNDAINFESNGNFIAPENAYKIVKKGDSTIMKSELEYGDCNTKCQTPIGAINSS MPFHNIHPLTIGECPKYVKSNRLVLATGLRNSPQGERRRKKRGLFGAIAGFIEGGWQ GMVDGWYGYHHSNEQGSGYAADKESTQKAIDGVTNKVNSIIDKMNTQFEAVGREF NNLERRIENLNKKMEDGFLDVWTYNAELLVLMENERTLDFHDSNVKNLYDKVRLQ LRDNAKELGNGCFEFYHRCDNECMESVRNGTYDYPQYSEEARLKREEISGVKLESIG TYQILSIYSTVASSLALAIMVAGLFLWMCSNGSLQCRICI (SEQ ID NO:52), wherein said sequence corresponds to SEQ ID NO:15 starting with amino acid D17, and additionally comprising a full length C-terminal sequence, wherein the numbering of said first N-terminal amino acid position (D17) refers to the amino acid position 17 as exemplarily given in SEQ ID NO 8, and wherein the full length C-terminal sequence corresponds to amino acids 527 to 568, more particular to amino acids 534 to 568, wherein the numbering of said amino acid positions refers to the amino acid positions 527 to 568, or more particular to amino acid positions 534 to 568, as exemplarily given in SEQ ID NO 47. Herein, this sequence, SEQ ID NO:52 and sequences having different C-terminal sequences may be referred to as a “variant of SEQ ID NO:15”.

It is thus further understood, that the term “variants of SEQ ID NOs 9 to 47” is preferably also directed to the sequences SEQ ID NOs: 9 to 47 starting with amino acid D17 and having the full length C-terminal sequence, and wherein said sequences SEQ ID No 9 to 46 starting with amino acid D17 and having the full length C-terminal sequence are given in Table C, and said sequence SEQ ID NO: 47 starting with amino acid D17 and having the full length C-terminal sequence is given in Table D.

The terms “variant sequences as set forth in SEQ ID NOs: 9 to 47” or “variant sequences of SEQ ID NOs: 9 to 47”, as used herein, preferably also relate to the sequences as set forth in Table B or Table C, respectively, and, additionally the sequence as set forth in Table D, respectively.

In a still more preferred embodiment of the invention, the H5N1 virus of a different clade comprises H5 protein (2) having
(a) the amino acids 87L, 113D, 126H, 145(−), 156R, 160F, 167T, and 181N, or
(b) the amino acids 87P, 113N, 126R, 145L, 160Y, 172T, 181H, 201E, 206I, 208K, 254T, 341G and 421K, or
(c) the amino acids 87L, 113N, 126R, 145L, 156G, 160Y, 172T, 181H, and 254V,
   and/or wherein such H5 protein (2) comprises a peptide that comprises:
i. any one of the amino acid sequences of SEQ ID NOs: 62 to 137 or 9 to 46;
ii. any peptide that has at least 85%, preferably at least 95%, even more preferably at least 96%, even more preferably at least 97%, even more preferably at least 98%, even more preferably at least 99%, most preferably 100% sequence homology to the polypeptide of i) and that comprises hemagglutinin inhibition in a standard hemagglutinin inhibition assay; or
iii. any part of the polypeptides of i) or ii) comprising at least 334 contiguous amino acids of any of such peptides of i) or ii) and wherein any of such peptide comprises hemagglutinin inhibition in a standard hemagglutinin inhibition assay,
   and/or wherein such H5 protein (2) consists of or comprises a contiguous amino acid sequence which has at least 95% even more preferably at least 96%, even more preferably at least 97%, even more preferably at least 98%, even more preferably at least 99%, most preferably 100% sequence identity with any one of the sequences as set forth in SEQ ID NOs: 62 to 137 or 9 to 46.

In a yet more preferred embodiment of the invention, the H5N1 virus of a different clade comprises a polynucleotide encoding a H5 protein (2) having:
(a) the amino acids 87L, 113D, 126H, 145(−), 156R, 160F, 167T, and 181N, or
(b) the amino acids 87P, 113N, 126R, 145L, 160Y, 172T, 181H, 201E, 206I, 208K, 254T, 341G and 421K, or
(c) the amino acids 87L, 113N, 126R, 145L, 156G, 160Y, 172T, 181H, and 254V,
   and/or wherein such H5 protein (2) comprises a peptide that comprises:
i. any one of the amino acid sequences of SEQ ID NOs: 62 to 137 or 9 to 46;
ii. any peptide that has at least 85%, preferably at least 95%, even more preferably at least 96%, even more preferably at least 97%, even more preferably at least 98%, even more preferably at least 99%, most preferably 100% sequence homology to the polypeptide of i) and that comprises hemagglutinin inhibition in a standard hemagglutinin inhibition assay; or
iii. any part of the polypeptides of i) or ii) comprising at least 334 contiguous amino acids of any of such peptides of i) or ii) and wherein any of such peptide comprises hemagglutinin inhibition in a standard hemagglutinin inhibition assay,
   and/or wherein such H5 protein (2) consists of or comprises a contiguous amino acid sequence which has at least 95% even more preferably at least 96%, even more preferably at least 97%, even more preferably at least 98%, even more preferably at least 99%, most preferably 100% sequence identity with any one of the sequences as set forth in SEQ ID NOs: 62 to 137 or 9 to 46.

More particular, the H5N1 virus of a different clade preferably comprises H5 protein (2) which consists of or comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 68, 73, 106, 111, 15, or 20, and wherein such H5 protein (2) comprising or consisting of the amino acid sequence set forth in SEQ ID NO: 73, 111, or 20 are in particular more preferred.

Preferably, the H5N1 virus of a different clade in particular comprises a polynucleotide encoding a H5 protein (2) which consists of or comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 68, 73, 106, 111, 15, or 20, and wherein such H5 protein (2) comprising or consisting of the amino acid sequence set forth in SEQ ID NO: 73, 111, or 20 are in particular more preferred.

In particular, the present invention is directed to the H5 protein (1) described herein for use in a method of treating or preventing infections
(A) with Subclade A H5N1 virus of North African origin, namely an infection with a H5N1 virus comprising a H5 protein (2) having the amino acids according to alternative (a) as described herein, in particular according to (a) of claim 15 or 16, or comprising a H5 protein according to claim 15, 16 or 17 relating to any one of the sequences as set forth in SEQ ID NOs: 62 to 72, 100 to 110, 9 to 19, or 95 or 96, 133 or 134, 42 or 43, or
(B) with Subclade B H5N1 virus of North African origin, namely an infection with a H5N1 virus comprising a H5 protein having the amino acids according to alternative (b) or (c) as described herein, in particular according to (b) or (c) of claim 15 or 16, or comprising a H5 protein according to claim 15, 16 or 17 relating to any one of the sequences as set forth in SEQ ID NOs: 82 to 94, 111 to 132, 20 to 41, or 97 to 99, 135 to 137, or 44 to 46.

Particularly, the present invention is directed to the H5 protein (1) described herein for use in a method of treating or preventing infections
(A) with Subclade A H5N1 virus of North African origin, namely an infection with a H5N1 virus comprising a polynucleotide encoding a H5 protein (2) having the amino acids according to alternative (a) as described herein, in particular according to (a) of claim 15 or 16, or an infection with a H5N1 virus comprising a polynucleotide encoding a H5 protein (2) according to claim 15, 16 or 17 relating to any one of the sequences as set forth in SEQ ID NOs: 62 to 72, 100 to 110, 9 to 19, or 95 or 96, 133 or 134, 42 or 43, or
(B) with Subclade B H5N1 virus of North African origin, namely an infection with a H5N1 virus comprising a polynucleotide encoding a H5 protein (2) having the amino acids according to alternative (b) or (c) as described herein, in particular according to (b) or (c) of claim 15 or 16, or an infection with a H5N1 virus comprising a polynucleotide encoding a H5 protein (2) according to claim 15, 16 or 17 relating to any one of the sequences as set forth in SEQ ID NOs: 82 to 94, 111 to 132, 20 to 41, or 97 to 99, 135 to 137, or 44 to 46.

According to a further embodiment, the present invention also relates to nucleic acid molecules, which code for any of the H5 proteins (1), as described supra, for use in a method of treating or preventing infections with H5N1 virus of a different clade. Preferably, those nucleic acid molecules are RNA, DNA or copy cDNA molecules. Thus, the present invention relates to a nucleic acid molecule, preferably a cDNA molecule coding for a H5 protein or any modified forms of H5 protein, including any deletion, substitution and/or insertion mutant of H5 protein, wherein those H5 proteins having the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted.

According to a further embodiment, the present invention also relates to a nucleic acid molecule, preferably a cDNA molecule coding for any part of the H5 protein (1), which means encoding for any peptide-fragment which shows antigenic properties in an standard hemagglutinin inhibition assay as described supra, and having at least the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted. Normally such nucleic acid molecules, which code for an antigenic part of H5 protein, comprise 600, 540, 480, 450, 420, 390, 360, 330 or most preferably 315 contiguous nucleotides of the nucleotide sequence that codes for the H5 protein as mentioned above, modified or non-modified, and which shows antigenic properties in a standard hemagglutinin inhibition assay as described herein.

Further embodiments of antigenic parts of the H5 protein (1) are described supra. It is in the common knowledge of a person skilled in the art to construct any such nucleic acid molecules, preferably cDNA molecules which codes for the antigenic part of the H5 protein as described supra. This also include but is not limited to the construction of nucleic acid molecules, preferably of cDNA molecules, which codes for antigenic parts of the H5 protein as mentioned above including deletion mutants of H5 protein, which comprises:
i. at least 105, 90, 75, 60, 48, 45, 39, 30, 27, or most preferably 24 contiguous amino nucleotides of the nucleotide sequence that surrounds and includes the coding sequence that codes for amino acid 223N; and
ii. at least 105, 90, 75, 60, 48, 45, 39, 30, 27, or most preferably 24 contiguous amino nucleotides of the nucleotide sequence that surrounds and includes the coding sequence that codes for modification 328K+, and
iii. wherein any of such antigenic part of H5 protein show hemagglutinin inhibition in a standard hemagglutinin inhibition assay as described in Example 2.

Preferably, those surrounding nucleotides of the nucleotides, which code for amino acids 223N and/or 328K+, coding for SEQ ID NO:2 or SEQ ID NO:5.

Furthermore preferred nucleic acid molecules encoding for the H5 protein (1) according to the invention are:
i. any of those mentioned supra encoding for the amino acid 223N and the modification 328K+;
ii. any of those mentioned supra encoding for the amino acid 94N/223N and the modification 328K+;
iii. any nucleic acid molecules of avian origin encoding for the amino acid 223N, and the modification 328K+, wherein avian origin means that the H5 sequence derived from a virus isolate that was originally isolated from poultry infected with avian influenza virus type 5; or
iv. any nucleic acid molecules of avian origin encoding for the amino acids 94N/223N and the modification 328K+, wherein avian origin means that the H5 sequence derived from a virus isolate that was originally isolated from poultry infected with avian influenza virus type 5; or.
v. any nucleic acid molecules of avian origin encoding for the amino acids 155N/223N and the modification 328K+, wherein avian origin means that the H5 sequence derived from a virus isolate that was originally isolated from poultry infected with avian influenza virus type 5; or
vi. any nucleic acid molecule encoding for H5 protein of avian origin having the amino acid 120N/155N/223N and the modification 328K+, wherein avian origin means that the H5 sequence derived from a virus isolate that was originally isolated from poultry infected with avian influenza virus type 5; or
vii. any nucleic acid molecule encoding for H5 protein having the modifications

94N/223N and the modification 328K+; or
viii. any nucleic acid molecule encoding for H5 protein having the modifications

94N/155N/223N and the modification 328K+; or;
ix. any nucleic acid molecule encoding for H5 protein having the modifications 94N/120N/155N/223N and the modification 328K+; or
x. any nucleic acid molecule encoding for H5 protein having the modifications 223N, the modification 328K+, and one or more of the following amino acid clusters selected from the group consisting of:
   a. aa 93-95: GNF
   b. aa 123-125: SDH
   c. aa 128-130: SSG
   d. aa 138-140: GSS
   e. aa 226-228: MDF
   f. aa 270-272: EVE
   g. aa 309-311: NKL; or
xi. any nucleic acid molecule encoding for H5 protein having the amino acid 223N, the modification 328K+, and one or more of the following amino acid clusters selected from the group consisting of:
   a. aa 93-95: GNF
   b. aa 128-130: SSG
   c. aa 138-140: GSS; or
xii. any nucleic acid molecule encoding for H5 protein having the amino acid sequence of SEQ ID NO:5.

Furthermore preferred H5 proteins (1) as provided herewith include the H5 proteins as described by Hoffmann et al, PNAS, vol. 106, no. 36, pp. 12915-12920 of Sep. 6, 2005, wherein that H5 proteins includes one or more of the modifications as described above, at least the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted. The disclosure of this reference shall be entirely included herein by reference. Thus according to a further embodiments, the present invention also relates to any nucleic acid molecule, preferably a cDNA molecule coding for any of such proteins described by Hoffmann et al, PNAS, vol. 106, no. 36, pp. 12915-12920 of Sep. 6, 2005, wherein that H5 proteins includes one or more of the modifications as described above, at least the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted.

Methods, of how to introduce any of the above-mentioned modifications within the nucleotide sequence, including the encoding sequence of the H5 protein of an influenza virus, are well known in the art. The genomic sequence of the entire influenza virus can be modified according to the invention, for example according to the methods described in U.S. Pat. No. 6,951,754, with further references.

Furthermore, there may be employed conventional molecular biology, microbiology, and recombinant DNA techniques within the skill of the art to modify a nucleic acid sequence coding for an antigen as described herein. Such techniques are explained fully in the literature. See, e.g., Sambrook et al., Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. 1994).

According to a further embodiment, the present invention also relates to a vector that comprises any of such nucleic acid molecules as described supra. In other words, the present invention relates to a vector that includes the coding sequence of any such H5 protein (1), or part thereof as described supra. Preferably, said vector is an expression vector, which allows the expression of any such H5 protein (1) or part thereof as described supra. Vectors according to the invention are those which are suitable for the transfection or infection of bacterial, yeast or animal cells, in vitro or in vivo.

Vectors and methods for making and/or using vectors (or recombinants) for expression can be by or analogous to the methods disclosed in: U.S. Pat. Nos. 4,603,112, 4,769,330, 5,174,993, 5,505,941, 5,338,683, 5,494,807, 4,722,848, 5,942,235, 5,364,773, 5,762,938, 5,770,212, 5,942,235, 382,425, PCT publications WO 94/16716, WO 96/39491, WO 95/30018, Paoletti, “Applications of pox virus vectors to vaccination: An update, “PNAS USA 93: 11349-11353, October 1996, Moss, “Genetically engineered poxviruses for recombinant gene expression, vaccination, and safety,” PNAS USA 93: 11341-11348, October 1996, Smith et al., U.S. Pat. No. 4,745,051, (recombinant baculovirus), Richardson, C. D. (Editor), Methods in Molecular Biology 39, “Baculovirus Expression Protocols” (1995 Humana Press Inc.), Smith et al., “Production of Human Beta Interferon in Insect Cells Infected with a Baculovirus Expression Vector”, Molecular and Cellular Biology, December, 1983, Vol. 3, No. 12, p. 2156-2165; Pennock et al., “Strong and Regulated Expression of Escherichia coli B-Galactosidase in Infect Cells with a Baculovirus vector, “Molecular and Cellular Biology March 1984, Vol. 4, No. 3, p. 399-406; EPAO 370 573, U.S. application No. 920,197, filed Oct. 16, 1986, EP Patent publication No. 265785, U.S. Pat. No. 4,769,331 (recombinant herpesvirus), Roizman, “The function of herpes simplex virus genes: A primer for genetic engineering of novel vectors,” PNAS USA 93:11307-11312, October 1996, Andreansky et al., “The application of genetically engineered herpes simplex viruses to the treatment of experimental brain tumors,” PNAS USA 93: 11313-11318, October 1996, Robertson et al. “Epstein-Barr virus vectors for gene delivery to B lymphocytes”, PNAS USA 93: 11334-11340, October 1996, Frolov et al., “Alphavirus-based expression vectors: Strategies and applications,” PNAS USA 93: 11371-11377, October 1996, Kitson et al., J. Virol. 65, 3068-3075, 1991; U.S. Pat. Nos. 5,591,439, 5,552,143, WO 98/00166, allowed U.S. application Ser. Nos. 08/675,556, and 08/675,566 both filed Jul. 3, 1996 (recombinant adenovirus), Grunhaus et al., 1992, “Adenovirus as cloning vectors,” Seminars in Virology (Vol. 3) p. 237-52, 1993, Ballay et al. EMBO Journal, vol. 4, p. 3861-65, Graham, Tibtech 8, 85-87, April, 1990, Prevec et al., J. Gen Virol. 70,42434, PCT WO 91/11525, Felgner et al. (1994), J. Biol. Chem. 269, 2550-2561, Science, 259: 1745-49, 1993 and McClements et al., “Immunization with DNA vaccines encoding glycoprotein D or glycoprotein B, alone or in combination, induces protective immunity in animal models of herpes simplex virus-2 disease”, PNAS USA 93: 11414-11420, October 1996, and U.S. Pat. Nos. 5,591,639, 5,589,466, and 5,580,859, as well as WO 90/11092, WO93/19183, WO94/21797, WO95/11307, WO95/20660, Tang et al., Nature and Furth et al. Analytical Biochemistry, relating to DNA expression vectors, inter alia. See also WO 98/33510; Ju et al., Diabetologia, 41: 736-739, 1998 (lentiviral expression system); Sanford et al., U.S. Pat. No. 4,945,050; Fischbach et al. (Intracel), WO 90/01543; Robinson et al., seminars in Immunology vol. 9, pp. 271-283 (1997), (DNA vector systems); Szoka et al., U.S. patent No. (method of inserting DNA into living cells); McCormick et al., U.S. Pat. No. 5,677,178 (use of cytopathic viruses); and U.S. Pat. No. 5,928,913 (vectors for gene delivery), as well as other documents cited herein, each of which is incorporated by reference herein.

A viral vector, for instance, selected from pig herpes viruses, such as Aujeszky's disease virus, porcine adenovirus, poxviruses, especially vaccinia virus, avipox virus, canarypox virus, and swinepox virus, as well as DNA vectors (DNA plasmids) are advantageously employed in the practice of the invention.

### Methods of Producing the 115 Proteins (1) According to the Present Invention

According to another aspect, the present invention provides methods of producing and/or recovering high amounts of recombinant H5 protein: i) by permitting infection of susceptible cells in culture with a recombinant viral vector containing H5 DNA coding sequences, wherein H5 protein is expressed by the recombinant viral vector, and ii) thereafter recovering the H5 protein from cell culture. High amounts of H5 protein means, but are not limited to, more than about 20 μg/mL cell culture, preferably more than about 25 μg/mL, even more preferred more than about 30 μg/mL, even more preferred more than about 40 μg/mL, even more preferred more than about 50 μg/mL, even more preferred more than about 60 μg/mL, even more preferred more than about 80 μg/mL, even more preferred more than about 100 μg/mL, even more preferred than about 150 μg/mL, most preferred more than about 190 μg/mL.

According to a preferred embodiment, the H5 protein (1) is recovered by harvesting the whole (i.e. intact) SF+ cells expressing the H5 protein.

Preferred cells are those susceptible for infection with an appropriate recombinant viral vector, containing a H5 DNA and expressing the H5 protein (1). Preferably the cells are insect cells, and more preferably, they include the insect cells sold under the trademark SF+ insect cells (Protein Sciences Corporation, Meriden, Conn.). Preferred cell cultures have a cell count between about 0.3-2.0×10⁶ cells/mL, more preferably from about 0.35-1.9×10⁶ cells/mL, still more preferably from about 0.4-1.8×10⁶ cells/mL, even more preferably from about 0.45-1.7×10⁶ cells/mL, and most preferably from about 0.5-1.5×10⁶ cells/mL.

Preferred viral vectors include baculovirus such as BaculoGold (BD Biosciences Pharmingen, San Diego, Calif.), in particular provided that the production cells are insect cells. Although the baculovirus expression system is preferred, it is understood by those of skill in the art that other expression systems will work for purposes of the present invention, namely the expression of H5 into the supernatant of a cell culture. Such other expression systems may require the use of a signal sequence in order to cause H5 expression into the media.

Appropriate growth media will also be determinable by those of skill in the art with a preferred growth media being serum-free insect cell media such as Excell 420 (JRH Biosciences, Inc., Lenexa, Kans.) and the like.

The recombinant viral vector containing the H5 DNA sequences has a preferred multiplicity of infection (MOI) of between about 0.03-1.5, more preferably from about 0.05-1.3, still more preferably from about 0.09-1.1, and most preferably from about 0.1-1.0, when used for the infection of the susceptible cells. Preferably the MOIs mentioned above relates to one mL of cell culture fluid. Preferably, the method described herein comprises the infection of 0.35-1.9×10⁶ cells/mL, still more preferably of about 0.4-1.8×10⁶ cells/mL, even more preferably of about 0.45-1.7×10⁶ cells/mL, and most preferably of about 0.5-1.5×10⁶ cells/mL with a recombinant viral vector containing a H5 DNA and expressing the H5 protein having a MOI (multiplicity of infection) of between about 0.03-1.5, more preferably from about 0.05-1.3, still more preferably from about 0.09-1.1, and most preferably from about 0.1-1.0.

The infected cells are then incubated over a period of up to ten days, more preferably from about two days to about ten days, still more preferably from about four days to about nine days, and most preferably from about five days to about eight days. Preferred incubation conditions include a temperature between about 22-32° C., more preferably from about 24-30° C., still more preferably from about 25-29° C., even more preferably from about 26-28° C., and most preferably about 27° C. Preferably, the SF+ cells are observed following inoculation for characteristic baculovirus-induced changes. Such observation may include monitoring cell density trends and the decrease in viability during the post-infection period. It was found that peak viral titer is observed 3-5 days after infection and peak H5 protein expression in the cells is obtained between days 5 and 8, and/or when cell viability decreases to less than 10%.

Thus, one aspect of the present invention provides a method of producing and/or recovering recombinant H5 protein, preferably in amounts described above, by i) permitting infection of a number of susceptible cells (see above) in culture with a recombinant viral vector with a MOI as defined above, ii) expressing H5 protein by the recombinant viral vector, and iii) thereafter recovering the H5 protein from the cells obtained between days 5 and 8 after infection and/or cell viability decreases to less than 10%. Preferably, the recombinant viral vector is a recombinant baculovirus containing H5 DNA coding sequences and the cells are SF+ cells. Additionally, it is preferred that the culture be periodically examined for macroscopic and microscopic evidence of contamination or for atypical changes in cell morphology during the post-infection period. Any culture exhibiting any contamination should be discarded.

For recovery of H5 protein (1) that will be used in an immunogenic or immunological composition such as a vaccine, the inclusion of an inactivation step is preferred in order to inactivate the viral vector.

An “immunogenic or immunological composition” refers to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein, “vaccine” refers to that term as it is used by those of skill in the art. More particularly, “vaccine” refers to an immunogenic composition that, when administered to an animal in need thereof, results in a reduction in the incidence of or severity of clinical signs of influenza infection up to an including the complete prevention of such clinical signs. Preferably, the reduction in incidence or severity is at least 10%, more preferably at least 20%, still more preferably at least 30%, even more preferably at least 40%, more preferably at least 50%, still more preferably at least 60%, even more preferably at least 70%, more preferably at least 80%, still more preferably at least 90%, even more preferably at least 95%, and most preferably 100% in comparison to an animal or group of animals that did not receive the compositions of the present invention but that were exposed to infectious levels of influenza virus that would normally result in influenza infection resulting in exhibiting clinical signs.

Thus, the present invention also relates to a method of producing and/or recovering recombinant H5 protein, preferably in amounts described above, by i) permitting infection of a number of susceptible cells (see above) in culture with a recombinant viral vector with a MOI as defined above, ii) expressing H5 protein by the recombinant viral vector, iii) recovering the H5 expressed in cells obtained between days 5 and 8 after infection and/or cell viability decreases to less than 10%, and iv) inactivating the recombinant viral vector.

Preferably, this inactivation is done either just before or just after the filtration step, with after the filtration step being the preferred time for inactivation. Any conventional inactivation method can be used for purposes of the present invention. Thus, inactivation can be performed by chemical and/or physical treatments. In preferred forms, the volume of harvest fluids is determined and the temperature is brought to between about 32-42° C., more preferably between about 34-40° C., and most preferably between about 35-39° C. Preferred inactivation methods include the addition of cyclized binary ethylenimine (BEI), preferably in a concentration of about 1 to about 20 mM, preferably of about 2 to about 10 mM, still more preferably of about 2 to about 8 mM, still more preferably of about 3 to about 7 mM, most preferably of about 5 mM. For example the inactivation includes the addition of a solution of 2-bromoethyleneamine hydrobromide, preferably of about 0.4M, which has been cyclized to 0.2M binary ethylenimine (BEI) in 0.3N NaOH, to the fluids to give a final concentration of about 5 mM BEI. Preferably, the fluids are then stirred continuously for 72-96 hours and the inactivated harvest fluids can be stored frozen at −40° C. or below or between about 1-7° C. After inactivation is completed a sodium thiosulfate solution, preferably at 1.0M is added to neutralize any residual BEI. Preferably, the sodium thiosulfate is added in equivalent amount as compared to the BEI added prior to for inactivation. For example, in the event BEI is added to a final concentration of 5 mM, a 1.0M sodium thiosulfate solution is added to give a final minimum concentration of 5 mM to neutralize any residual BEI.

Thus, one further aspect of the present invention relates to a method of producing recombinant H5 protein, preferably in amounts described above, by i) permitting infection of a number of susceptible cells (see above) in culture with a recombinant viral vector with a MOI as defined above, ii) expressing H5 protein by the recombinant viral vector, iii) recovering the H5 expressed in the cells obtained between days 5 and 8 after infection and/or cell viability decreases to less than 10%, and iv) inactivating the recombinant viral vector. Preferably, the recombinant viral vector is a baculovirus containing H5 DNA coding sequences and the cells are SF+ cells. Preferred inactivation steps are those described above. Preferably, inactivation is performed between about 35-39° C. and in the presence of 2 to 8 mM BEI, still more preferred in the presence of about 5 mM BEI.

According to one further aspect of the present invention, the method described above also includes a neutralization step after step iv). This step v) comprises adding of an equivalent amount of an agent that neutralizes the inactivation agent within the solution. Preferably, if the inactivation agent is BEI, addition of sodium thiosulfate to an equivalent amount is preferred. Thus, according to a further aspect, step v) comprises adding of a sodium thiosulfate solution to a final concentration of about 1 to about 20 mM, preferably of about 2 to about 10 mM, still more preferably of about 2 to about 8 mM, still more preferably of about 3 to about 7 mM most preferably of about 5 mM, when the inactivation agent is BEI.

In preferred forms and especially in forms that will use the recombinant H5 protein in an immunogenic composition such as a vaccine, each lot of harvested H5 protein will be tested for inactivation by passage in the anchorage dependent, baculovirus susceptible insect cells, such as Sf9 cells. In a preferred form of this testing, 150 cm² of appropriate cell culture monolayer is inoculated with 1.0 mL of inactivated H5 fluids and maintained at 25-29° C. for 14 days with at least two passages. At the end of the maintenance period, the cell monolayers are examined for cytopathogenic effect (CPE) typical of H5 baculovirus. Preferably, positive virus controls are also used. Such controls can consist of one culture of Sf9 cells inoculated with a non-inactivated reference H5 baculovirus and one flask of Sf9 cells that remain non-inoculated. After incubation and passage, the absence of virus-infected cells in the BEI treated viral fluids would constitute a satisfactory inactivation test. The control cells inoculated with the reference virus should exhibit CPE typical of H5 baculovirus and the non-inoculated flask should not exhibit any evidence of H5 baculovirus CPE. Alternatively, at the end of the maintenance period, the supernatant samples could be collected and inoculated onto a Sf9 96 well plate, which has been loaded with Sf9 cells, and then maintained at 25-29° C. for 5-6 days. The plate is then fixed and stained with anti-H5 antibody conjugated to FITC or any labeled antibody directed to baculovirus specific proteins (i.e. gp64). The absence of CPE, H5 expression, or expression of baculovirus specific proteins (i.e. gp64) in the BEI treated viral fluids constitutes a satisfactory inactivation test. The control cells inoculated with the reference virus should exhibit CPE and IFA activity and the non-inoculated flask should not exhibit any evidence of H5 baculovirus CPE and contain no IFA activity.

Thus a further aspect described herein, relates to an inactivation test for determining the effectiveness of the inactivation of the recombination viral vector expressing H5 protein (1), comprises the steps: i) contacting at least a portion of the culture fluid containing the recombinant viral vector with an inactivating agent, preferably as described above, ii) adding a neutralization agent to neutralize the inactivation agent, preferably as described above, and iii) determining the residual infectivity by the assays as described above.

After inactivation, the relative amount of recombinant H5 protein in a sample can be determined in a number of ways. Preferred methods of quantitation include SDS-PAGE densitometry, ELISA, and animal vaccination studies that correlate known quantities of vaccine with clinical outcomes (serology, etc.). When SDS-PAGE is utilized for quantitation, the sample material containing an unknown amount of recombinant H5 protein is run on a gel, together with samples that contain different known amounts of recombinant H5 protein. A standard curve can then be produced based on the known samples and the amount of recombinant H5 in the unknown sample can be determined by comparison with this standard curve. Because ELISAs are generally recognized as the industry standard for antigen quantitation, they are preferred for quantitation.

### Vaccines Comprising 115 Proteins (1) or Nucleic Acid Molecules or Vectors Coding for Those

The invention further provides a combination of (a) the H5 protein (1) described herein and (b) an inactivated Newcastle disease virus for use in a method of treating or preventing infections with H5N1 virus of a different clade, in particular for use in any method of treating or preventing infections with H5N1 virus of a different clade as described herein.

Said combination is also termed “the combination described herein” hereinafter.

According to the invention it is understood that the combination described herein is preferably included in a multivalent combination vaccine or the combination described herein is in particular directed to a combined vaccination, more particular to an administration of the H5 protein (1) described herein and of the inactivated Newcastle disease virus within a maximum of 24 hours to an animal, in particular poultry, or human being in need thereof.

Preferably, the inactivated Newcastle disease virus is an inactivated whole Newcastle disease virion.

In another preferred embodiment, the inactivated Newcastle disease virus is an inactivated Newcastle disease virus obtained by inactivation of a Newcastle disease virus comprising a RNA polynucleotide having at least 70%, preferably at least 80%, more preferably at least 90%, still more preferably at least 95% or in particular 100% sequence identity with a RNA copy of the polynucleotide set forth in SEQ ID NO: 139 (cDNA sequence of LaSota strain virus), which has been inactivated.

In particular, the inactivated Newcastle disease virus is an inactivated Newcastle disease LaSota strain virus.

In one preferred embodiment the inactivated Newcastle Disease Virus is a Newcastle Disease Virus which has been inactivated with a reagent selected from the group consisting of Formaldehyde, binary ethyleneimine (BEI), Beta-Propio-Lactone (BPL), and combinations thereof.

The amount of inactivated Newcastle disease virus in the combination described herein is preferably between 10² and 10¹⁰ equivalents of egg infectious doses (EID50), preferably between 10⁶ and 10⁹ EID50, in particular preferably between 10⁷ and 10⁹ EID50. The amount of the H5 protein (1) in the combination described herein is preferably the same as mentioned hereinafter.

The amount of the H5 protein (1) according to the invention is preferably between 10 and 1000 Hemagglutination units (HAU's) per dose, more preferably between 50 and 950 HAU's per dose, even more preferably between 100 and 900 HAU's per dose, even more preferably between 200 and 800 HAU's per dose, even more preferably between 300 and 700 HAU's per dose, still more preferably between 300 and 500 HAU's per dose.

According to a further aspect, the present invention relates to vaccines or pharmaceutical compositions in general, that comprises,
i. one or more of the H5 proteins (1) as described herein or the combination described herein;
ii. one or more of the nucleic acid molecules as described herein, coding for any such H5 proteins (1); and/or
iii. one or more of the vectors as described herein, including any such nucleic acid molecules and coding for any such H5 proteins (1) as described herein; and
iv. a pharmaceutical acceptable carrier and/or excipient.

The term “pharmaceutical composition” “Pharmaceutical/vaccine composition” as described herein, includes but is not limited to, vaccines for the reduction or prevention of an infection or to a composition of matter for the treatment and lessening of an infection.

The preparation of nucleic acid based vaccines, preferably cDNA vaccines, coding for influenza hemagglutinin are described for example in Deck et al, Vaccine 1997; 15(1):71-78; Ulmer et al., Science 1993; 259:1745-1749; Ulmer et al., Vaccine 1994; 12(16):1541-1544. Any of those methods can be used for the production of nucleic acid based vaccines, preferably cDNA vaccines, coding for an influenza H5 protein as described herein.

Moreover, a vaccine, which comprises H5 protein (1) or parts thereof as described herein, can be produced by conventional approaches, e.g. by recombinant expression techniques or by biochemical purification and separation techniques. Recombinant expression techniques, including the expression in insect cells are well known in the art, and described for example in Sambrook et al., Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. 1994). Further examples of well established recombinant expression systems are bacterial expression systems such as E. coli or B. subtilis, yeast-based expression systems such as S. cerevisiae or S. pombe, or mammalian cell expression systems such as the BHK-, CHO- and/or NS0-based expression systems. Such systems are well known in the art and generally available, e.g. commercially through Clontech Laboratories, Inc. 4030 Fabian Way, Palo Alto, Calif. 94303-4607, USA. Further expression strategies are for example described in Liischow et al., Vaccine no. 19 (2001), pp. 4249-4259, or Veit et al., PNAS vol. 103 (2006), pp. 8197-8202. Furthermore, recombinant adeno-associated virus systems are well established and for example described in U.S. Pat. No. 5,436,146 or WO200203872 with further references. Moreover, vaccinia (pox) virus based expression systems, for example as described in U.S. Pat. No. 6,265,183 with further references, are also well established and suitable to produce recombinant antigen(s), antigenic composition(s) as used according to the invention. Further suitable expression systems make use of recombinant popova viruses, such as SV40, fowl pox virus, pseudorabies viruses and retroviruses.

The relevant pharmaceutical/vaccine compositions as described herein, can also comprise inactivated virus which comprises H5 protein (1) as described herein, an apathogenic version of a live virus comprising H5 protein (1) as described herein, preparation and/or fragments of a virus, wherein said preparation and/or fragment comprise the H5 protein (1) as described herein.

The skilled person knows additional components which may be comprised in said compositions/vaccines together with antigen (see for example, Remington's Pharmaceutical Sciences. (1990). 18th ed. Mack Publ., Easton). The expert may use known injectable, physiologically acceptable sterile solutions. For preparing a ready-to-use solution, aqueous isotonic solutions, such as e.g. saline or corresponding plasma protein solutions, are readily available. The pharmaceutical composition/vaccine may be present as lyophylisates or dry preparations, which can be reconstituted with a known injectable solution directly before use under sterile conditions, e.g. as a kit of parts.

In addition the pharmaceutical/vaccine compositions of the present invention can include one or more veterinary-acceptable carriers. As used herein, “a veterinary-acceptable carrier” includes but is not limited to any and all solvents, dispersion media, coatings, adjuvants, stabilizing agents, diluents, preservatives, antibacterial and antifungal agents, isotonic agents, adsorption delaying agents, and the like.

Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

A preservative as used herein refers to an anti-microbiological active agent, such as for example Gentamycin, Merthiolate, and the like. In particular adding of a preservative is most preferred for the preparation of a multi-dose composition. Those anti-microbiological active agents are added in concentrations effective to prevent the composition of interest for any microbiological contamination or for inhibition of any microbiological growth within the composition of interest.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion.

The emulsion can be based in particular on light liquid paraffin oil (European Pharmacopoeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp 51-94 (1995) and Todd et al., Vaccine 15:564-570 (1997). Examples for suitable oil-in water emulsions are Emulsigen-based adjuvants, such as EMULSIGEN®, EMULSIGEN-D®, EMULSIGEN-P®, EMULSIGEN-75® (MVP Laboratories, Inc. Omaha, Nebr., USA). It has been surprisingly found, that pharmaceutical/vaccine compositions that comprise H5 protein, preferably recombinant H5 protein as described herein, have been effectively adjuvanted with oil-in water emulsions, preferably with such Emulsigen-based adjuvants, more preferably with EMULSIGEN® and EMULSIGEN-D®.

Moreover, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars orpolyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with allyl sucrose or with allyl pentaerythritol. Among then, there may be mentioned Carbopol 974P, 934P and 971P. Most preferred is the use of Carbopol 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, or muramyl dipeptide among many others.

Preferably, the adjuvant is added in an amount of about 100 μg to about 10 mg per dose. Even more preferred the adjuvant is added in an amount of about 100 μg to about 10 mg per dose. Even more preferred the adjuvant is added in an amount of about 500 μg to about 5 mg per dose. Even more preferred the adjuvant is added in an amount of about 750 μg to about 2.5 mg per dose. Most preferred the adjuvant is added in an amount of about 1 mg per dose.

The pharmaceutical/vaccine compositions can further include one or more other immunomodulatory agents such as, e.g., interleukins, interferons, or other cytokines. The pharmaceutical/vaccine compositions can also include Gentamicin and Merthiolate. While the amounts and concentrations of adjuvants and additives useful in the context of the present invention can readily be determined by the skilled artisan, the present invention contemplates compositions comprising from about 50 μg to about 2000 μg of adjuvant and preferably about 250 ug/1 ml dose of the vaccine composition. In another preferred embodiment, the present invention contemplates vaccine compositions comprising from about 1 ug/ml to about 60 μg/ml of antibiotics, and more preferably less than about 30 μg/ml of antibiotics.

Thus, according to a further embodiment, the present invention also relates to a pharmaceutical/vaccine composition comprising
i. a therapeutically effective amount of any one of the H5 proteins of influenza virus as described herein, wherein the H5 protein having the amino acid 223N and the modification 328K+, wherein numbering of the amino acid positions of the H5 protein refers to the amino acid position as exemplarily given in SEQ ID NO:2 and wherein the modification 328K+ means that at amino acid position 328 of H5 protein a second Lysine (K+) is inserted; and
ii. pharmaceutically acceptable adjuvants as described above.
   Preferably, the adjuvant is selected from the group consisting of:
a) EMULSIGEN®, an oil-in-water emulsion (o/w);
b) EMULSIGEN-D®, an oil-in-water (o/w) with dimethyldioctadecylammonum bromide (DDA);
c) a Polygen, a copolymer
d) EMULSIGEN-P®, an oil-in-water (o/w) with a proprietary immunostimulant
e) Carbigen is a cross-linked polymer
f) EMULSIGEN-75®, a double adjuvants comprise of an oil-in-water (o/w) with a cross-linked polymer
g) ISA 70 is a water-in-oil (w/o)

Most preferably, the adjuvants is an oil-in-water emulsion such as an emulsigen-based adjuvant selected from the group consisting of EMULSIGEN®, EMULSIGEN-D®, EMULSIGEN-P®, EMULSIGEN-75®, EMULSIGEN® and EMULSIGEN-P®. Most preferably EMULSIGEN® and EMULSIGEN-P® are used in the formulation of the current invention.

According to a further aspect, the pharmaceutical/vaccine compositions as provided herewith comprise one or more antigen. Preferably, that further antigen is an antigen of a poultry or mammalian pathogen. According to a further embodiments, that additional antigen is an further influenza antigen such as hemagglutinin H5, H7, H9, or any other hemagglutinin of influenza virus, wherein the H5 is preferably a H5 protein of a H5N1 virus of a clade different than clade 1, in particular of a H5N1 virus of North African origin, such as the H5 protein (2) described herein. The additional antigen(s) can be added in a purified form, as part of an antigenic preparation, in the form of a killed microorganism or in the form of a modified live microorganism.

The term “antigen”, as used herein means, but is not limited to, peptides, polypeptides, glycopeptides, or polysaccharides which are capable of specifically interacting with an antigen recognition molecule of the immune system, such as an immunoglobulin (antibody) or T cell antigen receptor in order to elicit, activate or stimulate an immune response directed to said antigen in a host to which said antigen is administered. The term “antigen” also refers to nucleic acid molecules, preferably DNA- or RNA-molecules, each of which codes for and express a peptide, polypeptide, or glycopeptide that is capable of specifically interacting with an antigen recognition molecule of the immune system, such as an immunoglobulin (antibody) or T cell antigen receptor in order to elicit, activate or stimulate an immune response against the antigen that is coded by the nucleic acid molecule. The antigen used for the preparation of the pharmaceutical composition which is used according to the invention is a microorganism or an antigenic part and/or preparation of said microorganism. In this connection, the term “immunization”, as used herein, means but is not limited to, any cause or enhancement of an immune response. The term “immune response” is already described supra.

Administration strategies for influenza vaccines are well known in the art. Mucosal vaccination strategies for inactivated and attenuated virus vaccines are contemplated. While the mucosa can be targeted by local delivery of a vaccine, various strategies have been employed to deliver immunogenic proteins to the mucosa.

In a specific embodiment, the vaccine can be administered in an admixture with, or as a conjugate or chimeric fusion protein with, cholera toxin, such as cholera toxin B or a cholera toxin A/B chimera (Hajishengallis, J. Immunol., 154:4322-32, 1995; Jobling and Holmes, Infect Immun., 60:4915-24, 1992). Mucosal vaccines based on use of the cholera toxin B subunit have been described (Lebens and Holmgren, Dev Biol Stand 82:215-27, 1994). In another embodiment, an admixture with heat labile enterotoxin (LT) can be prepared for mucosal vaccination.

Other mucosal immunization strategies include encapsulating the virus in microcapsules (U.S. Pat. No. 5,075,109, U.S. Pat. No. 5,820,883, and U.S. Pat. No. 5,853,763) and using an immunopotentiating membranous carrier (WO 98/0558) Immunogenicity of orally administered immunogens can be enhanced by using red blood cells (rbc) or rbc ghosts (U.S. Pat. No. 5,643,577), or by using blue tongue antigen (U.S. Pat. No. 5,690,938).

According to another aspect, the present invention relates to a method for preparing a pharmaceutical/vaccine composition as described above, preferably a method for producing a vaccine which comprises a recombinant, baculovirus expressed H5 protein as described supra. Generally, this method includes the steps of transfecting a construct into a virus, wherein the construct comprises i) recombinant H5 cDNA as described herein, ii) infecting cells in growth media with the transfected virus, iii) causing the virus to express the recombinant H5 protein as described herein iv) recovering the expressed H5 protein from the culture v) and preparing the composition by blending the expressed H5 protein with a suitable adjuvant and/or other pharmaceutically acceptable carrier.

Preferred adjuvants are those described above. Thus according to a further aspect, the method for preparing an antigenic composition, such as for example a vaccine, for invoking an immune response against influenza infections comprises i) preparing and recovering H5 protein, and ii) admixing this with a suitable adjuvants.

In addition, the vaccine composition of the present invention can also include diluents, isotonic agents, stabilizers, and/or preservatives. Diluents can include water, saline, dextrose, ethanol, glycerol, and the like. Isotonic agents can include as non-limiting examples inorganic or organic salts, e.g., sodium chloride, dextrose, mannitol, sorbitol, and lactose, saccharides, trehalose, mannitol, saccharose among others. Stabilizers include albumin and alkali salts of ethylendiamintetracetic acid, among others. Suitable adjuvants are those described above.

### Medicinal Use of any of Such 115 Proteins (1), Nucleic Acid Molecules, Vectors, Vaccines, and Combinations Described Herein

The H5 proteins (1) as provided herewith, the nucleic acid molecules coding for any such H5 proteins (1), the vectors comprising any such nucleic acid molecules coding for any such H5 proteins (1) as described herein, and any pharmaceutical/vaccine composition comprising any of such H5 protein (1), nucleic acid molecule or vector or the combination described herein can be used as a medicine, preferably for the treatment and prophylaxis of infections, caused by influenza virus, most preferably by influenza A virus. The H5 proteins (1) as provided herewith, the nucleic acid molecules encoding for any such H5 proteins, the vectors comprising any such nucleic acid molecules encoding for any such H5 proteins (1) as described herein, and any pharmaceutical/vaccine composition comprising any of such H5 protein (1), nucleic acid molecule or vector, as described herein, or the combination described herein can be used for the treatment or prophylaxis of human beings as well as in veterinary medicine. When used in veterinary medicine, the treatment of poultry, preferably bird, chicken, duck, turkey and the like as well as mammals, preferably pigs, cattle, horses, seals, camels, dogs, cats, hamsters, mice and the like, is preferred.

In terms of the present invention, “prophylaxis” refers to the reduction in the incidence of or severity of clinical signs of influenza infection up to an including the complete prevention of such clinical signs. Preferably, the reduction in incidence or severity is at least 10%, more preferably at least 20%, still more preferably at least 30%, even more preferably at least 40%, more preferably at least 50%, still more preferably at least 60%, even more preferably at least 70%, more preferably at least 80%, still more preferably at least 90%, even more preferably at least 95%, and most preferably 100% in comparison to an animal or group of animals that did not receive the compositions of the present invention but that were exposed to infectious levels of influenza virus that would normally result in influenza infection resulting in exhibiting clinical signs.

Thus, according to another aspect the present invention relates to the use of H5 proteins (1) as provided herewith, the nucleic acid molecules encoding for any such H5 proteins (1), the vectors comprising any such nucleic acid molecules encoding for any such H5 proteins (1) as described herein and any pharmaceutical/vaccine compositions comprising any of such H5 protein (1), nucleic acid molecule or vector as described herein or the combination described herein, can be used as a medicine, preferably as a medicine for human beings and/or as veterinary medicine, preferably for poultry, in particular for chicken.

Moreover, H5 proteins (1) as provided herewith, the nucleic acid molecules coding for any such H5 proteins (1), the vectors comprising any such nucleic acid molecules coding for any such H5 protein (1), as described herein, or the combination described herein can be used for the preparation of a pharmaceutical composition, as described herein, preferably of a single-shot vaccine or a one dose vaccine, for the prophylaxis or treatment of infections caused by H5N1 virus of a clade other than clade 1, wherein said H5N1 virus of a clade other than clade 1 is preferably the H5N1 virus of a different clade as described herein. As mentioned above, those pharmaceutical compositions/vaccine compositions can be used for the treatment and/or prophylaxis of human beings as well as for the treatment and/or prophylaxis of animals, such as poultry, preferably bird, chicken, duck, turkey and the like as well as mammals, preferably pigs, cattle, horses, seals, camels, dogs, cats, hamsters, mice and the like.

According to a further aspect, the present invention also relates to a method for the treatment or prophylaxis of influenza virus infections caused by H5N1 virus of a clade other than clade 1, wherein said H5N1 virus of a clade other than clade 1 is preferably the H5N1 virus of a different clade as described herein, wherein the method comprising administration of a therapeutically effective amount of the H5 protein (1) as described herein or of the combination described herein, to a subject in need of such a treatment. Moreover, the present invention also relates to a method for the treatment or prophylaxis of influenza virus infections caused by H5N1 virus of a clade other than clade 1, wherein said H5N1 virus of a clade other than clade 1 is preferably the H5N1 virus of a different clade as described herein, wherein the method comprising administration of a therapeutically effective amount of any H5 nucleic acid molecule or vector as described herein, that codes for any H5 protein (1) as described herein, to a subject in need of such a treatment. Furthermore, the present invention also relates to a method for the treatment or prophylaxis of influenza virus infections caused by H5N1 virus of a clade other than clade 1, wherein said H5N1 virus of a clade other than clade 1 is preferably the H5N1 virus of a different clade described herein, wherein the method comprising administration of a therapeutically effective amount of the vaccine comprising any such H5 protein (1), nucleic acid molecule or vector, as described herein, to a subject in need of such a treatment. The subject in need thereof can be a human being as well as an animal, preferably poultry, even more preferably bird, chicken, duck, turkey or a mammal, preferably pig, cattle, horse, seal, camel, dog, cat, hamster, mouse and the like.

Preferably, the administration, as described herein, is a single-shot administration or a one dose administration.

Preferably, when chicken are vaccinated, the H5 protein as described herein can be used for vaccination at day 1 of age or later, e.g. at day 10, or at day 1 to 10, or at day 10 or later.

Preferably the influenza infection that can be treated by the administration of any H5 protein (1), the nucleic acid molecule or vector encoding for any such H5 protein, or any pharmaceutical/vaccine compositions as described herein, is caused by H5N1 virus of a clade other than clade 1, wherein said H5N1 virus of a clade other than clade 1 is preferably the H5N1 virus of a different clade as described herein and, as the case may be, also in combination with another avian, swine or human influenza virus or any combination or hybrid thereof.

A further advantage of the present invention is that it benefits a “DIVA” (Differentiation of Infected and Vaccinated Animals) concept with specific Elisa Kits for differentiating between vaccinated human beings or animals and human beings or animals infected with H5N1 virus.

According to another aspect, the present invention relates to a kit of parts, that comprises i) any of such H5 protein (1) as described herein, the nucleic acid molecule or vector encoding for any such H5 protein, or any pharmaceutical/vaccine composition comprising any of such H5 protein, nucleic acid molecule or vector as described herein, and ii) a package leaflet indicating the use of such H5 protein, nucleic acid molecule, vector or vaccine for the treatment or prophylaxis of infections caused by H5N1 virus of a clade other than clade 1, wherein said H5N1 virus of a clade other than clade 1 is preferably the H5N1 virus of a different clade as described herein. When chicken are vaccinated, the H5 protein (1) as described herein can be used for vaccination at day 1 on age or later.

It is thus understood that the kit of parts as mentioned herein is for the use, or is used, respectively, for the treatment or prophylaxis of infections caused by H5N1 virus of a clade other than clade 1, wherein said H5N1 virus of a clade other than clade 1 is preferably the H5N1 virus of a different clade as described herein.

According to a further embodiment, that kit in parts comprises at least one further antigen of poultry or mammalian pathogen and the information indication the medicinal, human or veterinary use of that additional antigen, in particular the further antigen as mentioned above.

The invention further provides a method for reducing viral shedding in a subject, comprising administering the H5 protein (1) described herein or the combination as described herein to a subject infected with or at risk of a viral infection with H5N1 virus of a clade other than clade 1, wherein said H5N1 virus of a clade other than clade 1 is preferably the H5N1 virus of a different clade as described herein.

The invention also relates to the H5 protein (1) described herein or the combination as described herein for use in a method for reducing viral shedding in a subject, wherein said H5 protein (1) or said combination is to be administered to a subject infected with or at risk of a viral infection with H5N1 virus of a clade other than clade 1, and wherein said H5N1 virus of a clade other than clade 1 is preferably the H5N1 virus of a different clade as described herein.

Also, the invention provides the use of the H5 protein (1) described herein or of the combination as described herein for the preparation of a medicament for reducing viral shedding in a subject infected with or at risk of a viral infection with H5N1 virus of a clade other than clade 1, wherein said H5N1 virus of a clade other than clade 1 is preferably the H5N1 virus of a different clade as described herein.

Preferably, the H5 protein (1) according to the invention, the combination described herein, the vaccine as described herein or the kit mentioned herein is for use as a single-shot vaccine or in a one-dose vaccination.

### EXAMPLES

The following examples set forth preferred materials and procedures in accordance with the present invention. It is to be understood, however, that these examples are provided by way of illustration only, and nothing therein should be deemed a limitation upon the overall scope of the invention.

### Example 1

### Construction of a Recombinant Baculoviruses Coding for and Expressing HA 115 Antigens

The recombinant baculovirus containing the H5 HA antigen was generated as follows: the coding sequences of the H5 HA (SEQ ID NO:3) was chemically synthesized and subcloned into the transfer vector pVL1392 (BD Biosciences Pharmingen, San Diego, Calif.). The H5 HA MutK+(SEQ ID NO:5) was generated by using oligonucleotide primers and the QUIKCHANGE® Site-Directed Mutagenesis Kit (Stratagene, La Jolla, Calif.) and subcloned into the transfer vector pVL1392 (BD Biosciences Pharmingen, San Diego, Calif.). The pVL1392 plasmids containing the genes coding for H5 HA antigen (SEQ ID NO:3) and H5 HA MutK+(SEQ ID NO:5) were then co-transfected with DIAMONDBAC® (Sigma) baculovirus DNA into Sf9 insect cells (BD Biosciences Pharmingen) to generate the recombinant baculovirus containing the genes H5 HA coding for SEQ ID NO:3 and H5 HA mutK+ coding for SEQ ID NO:5. The recombinant baculoviruses containing the genes coding for H5 HA (SEQ ID NO:3) and H5 HA MutK+(SEQ ID NO:5) were plaque-purified and Master Seed Viruses (MSVs) were propagated on the SF+ cell line, aliquoted, and stored at −70° C. Insect cells infected with H5 HA baculoviruses as described above to generate MSV or Working Seed Viruses express H5 HA antigen (SEQ ID NO:3) and H5 HA MutK+ (SEQ ID NO:5) antigen as detected by polyclonal serum or monoclonal antibodies in an indirect fluorescent antibody assay or Western blot.

After being seeded with the appropriate amounts of recombinant baculoviruses (H5 HA and H5 HA MutK+, respectively), spinner flasks containing SF+ cells (Protein Sciences, Inc., Meriden, Conn.) were then incubated at 27±2° C. for 7 days and with stirring 100 rpm during that time. The flasks used ventilated caps to allow for air flow. The crude whole cell culture containing baculovirus infected SF+ cells and the cell culture supernatants of each culture were harvested.

### Example 2

### Preparation Of Pharmaceutical Compositions (Vaccines) Comprising HA H5 Antigens

The crude whole cell H5 HA protein and H5 HA Mutk+ protein expressed in insect cells by baculovirus-based expression system were harvested. Baculoviruses were inactivated in the presence of 5 mM cyclized binary ethylenimine (BEI) (final concentration) between about 32 and 39° C. for 72 to 96 hours. After inactivation is completed, a 0.3 M sodium thiosulfate solution was added to a final concentration of 5 mM to neutralize any residual BEI. After neutralization, various adjuvants were added and the following vaccine/pharmaceutical compositions were generated.

### Vaccines

| | |
|---|---|
| Generic | 501 |
| product | |
| name | |
| Antigen | Crude whole-cell H5 HA protein expressed in insect cells by |
| | a baculovirus-based expression system. |
| Formula- | An experimental vaccine comprised of cultured insect cells |
| tion | and supernatant expressing recombinant H5 HA. The vaccine |
| | was adjuvanted with Emulsigen. |
| Generic | 502 |
| product | |
| name | |
| Antigen | Crude whole-cell H5 HA protein expressed in insect cells by |
| | a baculovirus-based expression system. |
| Formula- | An experimental vaccine comprised of cultured insect cells |
| tion | and supernatant expressing recombinant H5 HA. The vaccine |
| | was adjuvanted with Emulsigen-D. |
| Generic | 503 |
| product | |
| name | |
| Antigen | Crude whole-cell H5 HA protein expressed in insect cells by |
| | a baculovirus-based expression system. |
| Formula- | An experimental vaccine comprised of cultured insect cells |
| tion | and supernatant expressing recombinant H5 HA. The vaccine |
| | was adjuvanted with Polygen. |
| Generic | 504 |
| product | |
| name | |
| Antigen | Crude whole-cell H5 HA protein expressed in insect cells by |
| | a baculovirus-based expression system. |
| Formula- | An experimental vaccine comprised of cultured insect cells |
| tion | and supernatant expressing recombinant H5 HA. The vaccine |
| | was adjuvanted with Emulsigen-P. |
| Generic | 505 |
| product | |
| name | |
| Antigen | Crude whole-cell H5 HA protein expressed in insect cells by |
| | a baculovirus-based expression system. |
| Formula- | An experimental vaccine comprised of cultured insect cells |
| tion | and supernatant expressing recombinant H5 HA. The vaccine |
| | was adjuvanted with Carbigen. |
| Generic | 506 |
| product | |
| name | |
| Antigen | Crude whole-cell H5 HA protein expressed in insect cells by |
| | a baculovirus-based expression system. |
| Formula- | An experimental vaccine comprised of cultured insect cells |
| tion | and supernatant expressing recombinant H5 HA. The vaccine |
| | was adjuvanted with Emulsigen-75. |
| Generic | 507 |
| product | |
| name | |
| Antigen | Crude whole-cell H5 HA protein expressed in insect cells by |
| | a baculovirus-based expression system. |
| Formula- | An experimental vaccine comprised of cultured insect cells |
| tion | and supernatant expressing recombinant H5 HA. The vaccine |
| | was adjuvanted with ISA 70. |
| Generic | 508 |
| product | |
| name | |
| Antigen | Crude whole-cell H5 HA mutK+ protein expressed in insect |
| | cells by a baculovirus-based expression system. |
| Formula- | An experimental vaccine comprised of cultured insect cells |
| tion | and supernatant expressing recombinant H5 HA. The vaccine |
| | was adjuvanted with Emulsigen. |
| Generic | 509 |
| product | |
| name | |
| Antigen | Crude whole-cell H5 HA mutK+ protein expressed in insect |
| | cells by a baculovirus-based expression system. |
| Formula- | An experimental vaccine comprised of cultured insect cells |
| tion | and supernatant expressing recombinant H5 HA. The vaccine |
| | was adjuvanted with Emulsigen-D. |
| Generic | 510 |
| product | |
| name | |
| Antigen | Crude whole-cell H5 HA mutK+ protein expressed in insect |
| | cells by a baculovirus-based expression system. |
| Formula- | An experimental vaccine comprised of cultured insect cells |
| tion | and supernatant expressing recombinant H5 HA. The vaccine |
| | was adjuvanted with Polygen. |
| Generic | 511 |
| product | |
| name | |
| Antigen | Crude whole-cell H5 HA mutK+ protein expressed in insect |
| | cells by a baculovirus-based expression system. |
| Formula- | An experimental vaccine comprised of cultured insect cells |
| tion | and supernatant expressing recombinant H5 HA. The vaccine |
| | was adjuvanted with Emulsigen-P. |
| Generic | 512 |
| product | |
| name | |
| Antigen | Crude whole-cell H5 HA mutK+ protein expressed in insect |
| | cells by a baculovirus-based expression system. |
| Formula- | An experimental vaccine comprised of cultured insect cells |
| tion | and supernatant expressing recombinant H5 HA. The vaccine |
| | was adjuvanted with Carbigen. |
| Generic | 513 |
| product | |
| name | |
| Antigen | Crude whole-cell H5 HA mutK+ protein expressed in insect |
| | cells by a baculovirus-based expression system. |
| Formula- | An experimental vaccine comprised of cultured insect cells |
| tion | and supernatant expressing recombinant H5 HA. The vaccine |
| | was adjuvanted with Emulsigen-75. |
| Generic | 514 |
| product | |
| name | |
| Antigen | Crude whole-cell H5 HA K+ protein expressed in insect cells |
| | by a baculovirus-based expression system. |
| Formula- | An experimental vaccine comprised of cultured insect cells |
| tion | and supernatant expressing recombinant H5 HA. The vaccine |
| | was adjuvanted with ISA 70. |

### Example 3

### Vaccination of Chicken Against Avian Influenza

A combination vaccine comprising H5 HA Mutk+ (Fraction 1) and inactivated Newcastle disease virus (Fraction 2), named “BACULO AI+ND KV” has been evaluated in animal trials. The vaccine was formulated with the haemmagglutinin H5 produced in the Baculovirus expression system based on the MutK+ construct (Examples 1 and 2). The origin of the Newcastle Disease (ND) virus fraction is the whole virus.

Fraction 1: Avian Influenza (AI) fraction.

Recombinant, baculovirus-expressed, H5 hemagglutinin (H5 HA) from Avian Influenza H5N1 virus. AI fraction is inactivated with binary ethyleneimine (BEI). No residual infectivity coming from Baculovirus vector is allowed.

Fraction 2: Newcastle Disease Fraction.

Whole virion, Newcastle Disease Virus (ND), LaSota Strain. ND fraction is inactivated with Formaldehyde, BEI or Beta-Propio-Lactone (BPL). No residual infectivity coming from ND virus is allowed.

Formula Composition:

Inactivated harvest material from H5 HA protein and ND are blended into a water/oil emulsion. The mixture includes mineral oil as an adjuvant.

For evaluation of vaccine efficacy, three clinical parameters were considered: 1) Morbility/mortality. 2) Antibodies levels. 3) Viral shedding.

In all studies SPF chickens were vaccinated, administration of the vaccine was by subcutaneous route, in the back of the neck. A dose of 0.5 ml was administered unless otherwise stated.

Chickens were maintained inside isolator units during the whole duration of the studies. Studies were compliant with OIE international guidelines for evaluation of Avian Influenza vaccines.

Challenge was conducted to evaluate the Avian Influenza (AI) antigenic fraction. Chickens were inoculated 3 weeks after vaccination by the intra-nasal (50 n1) and oral (50 n1) route administering a total of 100 n1 of allantoic fluid containing 10⁶ EID₅₀ of the challenge virus.

To evaluate protection from challenge against HPAI H5N1 two studies were conducted:

(1) Protectotypes study: Using a single or double vaccination (evaluating boosting effect), ages of 1 day old or 10 days old chickens (evaluating age effect), and doses of 0.5 or 0.2 ml (evaluating dose effect).

Two different challenge strains were used for this study: a) A subclade 2.3.2 Vietnamese strain (isolated in 2006) which has been recently causing disease in South-East-Asia (China, Vietnam) Poultry production. b) A subclade 2.2.1 group B1 Egyptian strain (isolated in 2010), which has been recently causing disease in Egyptian Poultry production. Challenge strains are not genetically close to the vaccine baculovirus construct (MutK+). Results are interpreted in the context of protectotypes as broadening up the protection conferred for two immunizations with similar or different vaccines.

Conclusions Protectotypes Study:

1) Protection between 80 and 100% was observed depending on the age or dose. 100% protection was observed when administered as 0.5 ml dose at 10 days old of the bivalent formulation.

2) When administered as a single 0.5 ml immunization of BACULO AI+ND KV at 10 days of age, the same protection is observed than administering two shots of the inactivated traditionally-produced commercial Volvac AI KV vaccine.

3) When administered as a single 0.5 ml immunization of BACULO AI+ND KV at 10 days of age, similar level of H5-specific antibodies were detected in comparison with administering two shots of the inactivated traditionally-produced commercial Volvac AI KV vaccine.

4) Low levels of viral shedding were observed until 3 days post-challenge, when the vaccine was administered as a single 0.5 ml immunization of BACULO AI+ND KV at 10 days of age.

(2) BACULO efficacy study: using a single, unique vaccination at 10 days of age.

Three different challenge strains were used for this study: a) A subclade 2.2.1 Egyptian strain (isolated in 2008). b) A subclade 2.2.1 group A1 Egyptian strain (isolated in 2010). c) A subclade 2.2.1 group B1 Egyptian strain (isolated in 2010). The last two have been recently causing disease in Egyptian Poultry production.

Conclusions BACULO efficacy study:

1) Protection between 90 and 100% was observed.

2) Vaccine BACULO AI+ND KV showed performance compliant with European Medicine Agency (EMA) guidelines for vaccines against HPAI virus in birds.

3) This is the first report available demonstrating efficacy with a single shot administration for a baculovirus-based vaccine including a hemagglutinin genetically distant from those of the viruses used for challenge.

### Example 4

(1) Experimental design: This experiment was designed and conducted similar to the above described Example 3: For evaluation of vaccine efficacy, three clinical

parameters were considered: 1) Morbility/mortality. 2) Antibodies levels. 3) Viral shedding.

In all studies SPF chickens were vaccinated, the administration of the vaccine was by subcutaneous route, in the back of the neck. A vaccine prototype containing a clade 1 H5 protein was used (called Mut K+) formulated as a bivalent product with a second, ND (Newcastle disease virus) antigenic fraction.

A dose of 0.5 ml was administered unless otherwise stated Animals were vaccinated at 10 days of age.

Chickens were maintained inside isolator units during the whole duration of the studies. Studies were compliant with OIE international guidelines for evaluation of Avian Influenza vaccines.

Challenge was conducted to evaluate the Avian Influenza (AI) antigenic fraction. Chickens were inoculated 3 weeks after vaccination by the intra-nasal (50 μl) and oral (50 μl) route administering a total of 100 μl of allantoic fluid containing 10⁶ EID₅₀ of the challenge virus.

This is also summarized in the table (Table a) below (Vaccination was performed at 10 days of age, column 1 (ID of experimental groups according to the vaccine applied), column 2 (Vaccine dose), and column 3 (Challenge age)).

Challenge virus was A/Chicken/Egypt/1063/2010, which is classified as subclade 2.2.1.1 HP AIV H5N1 subtype. This is the official challenge strain used in Egypt for evaluation of vaccine batches. The challenge dose was 10⁶ EID₅₀.

(2) Results & Data analysis: Results & Data analysis are summarized in the table below (Table a): Column 4 (HI GMT (Geometric Mean Titer) 3 weeks post-vaccination, pre-challenge), column 5 (Percentage of survival, 2 weeks post-challenge), and column 6 (Detection of viral shedding, RT-PCR positive samples).

Results & Data analysis are summarized in the table below (Table a): Column 4 (HI GMT (Geometric Mean Titer) 3 weeks post-vaccination, pre-challenge), column 5 (Percentage of survival, 2 weeks post-challenge), and column 6 (Detection of viral shedding, RT-PCR positive samples).

TABLE a
Summary of the experimental design and of the results and data analysis of
Example 4.

| Experimental | | | | | | |
|---|---|---|---|---|---|---|
| group | | | | | Percentage | |
| (10 | | Challenge | GMT measured at | | of | Viral Shedding- |
| chickens | | dose | 31 days of age | | survival | Detection of |
| each) | Vaccine | (age) | Homologous | Heterologous | post- | viral RNA using |
| -Vaccine | Dose | -Strain | (vaccine | (challenge | challenge | RT-PCR- |
| ID- | (age) | 1063- | strain) | virus) | (%) | (#positives/total) |
| Mut K+ | 0.5 ml | 10⁶ | 9.1 | 0.9 | 100 | 2/10 |
| No vaccine | (10 days | EID₅₀ | — | — | 0 | 10/10 |
| | of age) | (31 days | | | | |
| | | of age) | | | | |

(3) Conclusions:

The vaccinated group survived the challenge. The vaccine prototype triggered an efficient immune response, as measured as HI titration using the homologous antigen.

The Mut K+ vaccine prototype provided good virological protection, as measured as ability to reduce viral shedding. RT-PCR Ct values were far too low to represent infectious virus but only residual genetic material.

### Example 5

(1) Experimental Design: The described experiment is designed to show the efficacy of a vaccine prototype only containing a clade 1 H5 protein (called Mut K+).

For evaluation of vaccine efficacy, three clinical parameters are considered: 1) Morbility/mortality. 2) Antibodies levels. 3) Viral shedding.

In all studies SPF chickens are vaccinated, the administration of the vaccine is by the subcutaneous route, in the back of the neck. A vaccine prototype containing a clade 1 H5 protein is used (called Mut K+) formulated as a monovalent product.

A dose of 0.5 ml is administered unless otherwise stated. Animals are vaccinated at 10 days of age.

Chickens are maintained inside isolator units during the whole duration of the studies. Studies are compliant with OIE international guidelines for evaluation of Avian Influenza vaccines.

Challenge is conducted to evaluate the efficacy of the vaccine in regard to the Avian Influenza (AI) antigenic fraction. Chickens are inoculated 3 weeks after vaccination by the intra-nasal (50 μl) and oral (50 μl) route administering a total of 100 μl of allantoic fluid containing 10⁶ EID₅₀ of the challenge virus.

This is also summarized in the tables below (See Table b and Table c). Vaccination was performed at 10 days of age. Column 1 (ID of experimental groups according to the vaccine applied), column 2 (Vaccine dose), and column 3 (Challenge age)).

Challenge virus is either A/Chicken/Egypt/1063/2010 (classified as subclade B or subclade 2.2.1.1 HP AIV H5N1 subtype) or A/chicken/Egypt/3982-8/2010 (classified as subclade A or subclade 2.2.1 HP AIV H5N1 subtype)

The A/Chicken/Egypt/1063/2010 was the official challenge strain used in Egypt for evaluation of vaccine batches until the end of 2012. The challenge dose is in any case 10⁶ EID₅₀.

TABLE b
Summary of the experimental design of Example 5.

| 1 | | |
|---|---|---|
| Experimental group | 2 | 3 |
| (10 chickens each) | Vaccine Dose | Challenge dose (age) |
| -Vaccine ID- | (age) | -Strain 1063- |
| Mut K+ | 0.5 ml | 10⁶ EID₅₀ |
| No vaccine | (10 days of age) | (31 days of age) |

TABLE c
Summary of the experimental design of Example 5.

| 1 | | |
|---|---|---|
| Experimental group | 2 | 3 |
| (10 chickens each) | Vaccine Dose | Challenge dose (age) |
| -Vaccine ID- | (age) | -Strain 3982-8 |
| Mut K+ | 0.5 ml | 10⁶ EID₅₀ |
| No vaccine | (10 days of age) | (31 days of age) |

(2) Conclusions:

The chickens of the vaccinated group (Table c) survive the challenge with A/chicken/Egypt/3982-8/2010 and nearly all chickens of the vaccinated group (Table b) survive the challenge with A/Chicken/Egypt/1063/2010. The vaccine prototype induces an efficient immune response, as measured by determination of the HI titer using the homologous antigen.

The Mut K+ vaccine prototype provides sufficient protection in regard to shedding of the challenge virus, as measured as ability to reduce viral shedding. The RT-PCR Ct values are very low and represent rather only residual virus genetic material instead infectious virus.

In the sequence listing (SEQ ID NOs: 1 to 50 and 139):

SEQ ID NO: 1 corresponds to H5 of A/Hong Kong/213/2003(H5N1) without signal peptide,
SEQ ID NOs: 2-7 correspond to SEQ ID NOs: 1-6 of the international (PCT) application number PCT/US2007/082699,
SEQ ID NO: 8 corresponds to H5 sequence of H5N1 “1709-6”,
SEQ ID NO: 9 corresponds to H5 sequence of H5N1 “1553-1/A1”,
SEQ ID NO: 10 corresponds to H5 sequence of H5N1 “1553-15/A1”,
SEQ ID NO: 11 corresponds to H5 sequence of H5N1 “2095-50/A1”,
SEQ ID NO: 12 corresponds to H5 sequence of H5N1 “3982-2/A1”,
SEQ ID NO: 13 corresponds to H5 sequence of H5N1 “3982-5/A1”,
SEQ ID NO: 14 corresponds to H5 sequence of H5N1 “3982-7/A1”,
SEQ ID NO: 15 corresponds to H5 sequence of H5N1 “3982-8/A1”,
SEQ ID NO: 16 corresponds to H5 sequence of H5N1 “3982-9/A1”,
SEQ ID NO: 17 corresponds to H5 sequence of H5N1 “3982-12/A1”,
SEQ ID NO: 18 corresponds to H5 sequence of H5N1 “3982-20/A1”,
SEQ ID NO: 19 corresponds to H5 sequence of H5N1 “3982-44/A1”,
SEQ ID NO: 20 corresponds to H5 sequence of H5N1 “1553-2B1”,
SEQ ID NO: 21 corresponds to H5 sequence of H5N1 “1553-6/B1”,
SEQ ID NO: 22 corresponds to H5 sequence of H5N1 “1553-13/B2”,
SEQ ID NO: 23 corresponds to H5 sequence of H5N1 “1553-26/B2”,
SEQ ID NO: 24 corresponds to H5 sequence of H5N1 “1553-28/B1”,
SEQ ID NO: 25 corresponds to H5 sequence of H5N1 “2095-39/B2”,
SEQ ID NO: 26 corresponds to H5 sequence of H5N1 “2095-46/B1”,
SEQ ID NO: 27 corresponds to H5 sequence of H5N1 “2095-49/B1”,
SEQ ID NO: 28 corresponds to H5 sequence of H5N1 “2095-65/B1”,
SEQ ID NO: 29 corresponds to H5 sequence of H5N1 “2095-68/B2”,
SEQ ID NO: 30 corresponds to H5 sequence of H5N1 “2095-70/B2”,
SEQ ID NO: 31 corresponds to H5 sequence of H5N1 “2095-73/B2”,
SEQ ID NO: 32 corresponds to H5 sequence of H5N1 “2095-75/B2”,
SEQ ID NO: 33 corresponds to H5 sequence of H5N1 “3982-3/B1”,
SEQ ID NO: 34 corresponds to H5 sequence of H5N1 “3982-4/B1”,
SEQ ID NO: 35 corresponds to H5 sequence of H5N1 “3982-13/B1”,
SEQ ID NO: 36 corresponds to H5 sequence of H5N1 “3982-14/B2”,
SEQ ID NO: 37 corresponds to H5 sequence of H5N1 “3982-19/B3”,
SEQ ID NO: 38 corresponds to H5 sequence of H5N1 “3982-21/B2”,
SEQ ID NO: 39 corresponds to H5 sequence of H5N1 “3982-43/B1”,
SEQ ID NO: 40 corresponds to H5 sequence of H5N1 “3982-50/B1”,
SEQ ID NO: 41 corresponds to H5 sequence of H5N1 “3982-52/B1”,
SEQ ID NO: 42 corresponds to H5 sequence of H5N1 “3982-55/A1”,
SEQ ID NO: 43 corresponds to H5 sequence of H5N1 “3982-56/A1”,
SEQ ID NO: 44 corresponds to H5 sequence of H5N1 “3982-78/B2”,
SEQ ID NO: 45 corresponds to H5 sequence of H5N1 “4794-17/B”,
SEQ ID NO: 46 corresponds to H5 sequence of H5N1 “4794-18/B”,
SEQ ID NO: 47 corresponds to H5 sequence translated from SEQ ID NO: 50,
SEQ ID NO: 48 codes for a H5 sequence of H5N1 “3982-8/A1” (SEQ ID NO: 15),
SEQ ID NO: 49 codes for a H5 sequence of H5N1 “1553-2/B1” (SEQ ID NO: 20),
SEQ ID NO: 50 corresponds to the consensus sequence obtained after analysis of the 38 H5 HA gene sequences coding for SEQ ID NOs: 9 to 46,
SEQ ID NO: 139 corresponds to the cDNA of Newcastle Disease Virus LaSota strain.

TABLE A
The following list is an alignment of the sequences as set forth in SEQ
ID Nos: 9 to 46, wherein the single letter code for amino acids is used:

| | ....\|....\| | ....\|....\| | ....\|....\| | ....\|....\| | ....\|....\| | ....\|....\| |
|---|---|---|---|---|---|---|
| | 5 | 15 | 25 | 35 | 45 | 55 |
| 1553-1/A1 | MEKIMLLLAI | VSLVKSDQIC | IGYHANNSTE | QVDTIMEKNV | TVTHAQDILE | KTHNGKLCNL |
| 1553-15/A1 | MEKIVLLLAI | VSIVKSDQIC | IGYHANNSTE | QVDTIMEKNV | TVTHAQDILE | KTHNGKLCNL |
| 2095-50/A1 | MEKIVLLLAI | VSLVKGDQIC | IGYHANNSTE | QVDTIMEKNV | TVTHAQDILE | KTHNGKLCNL |
| 3982-2/A1 | -----XLLAI | VSIVKSDQIC | IGYHANNSTE | QVDTIMEKNV | TVTHAQDILE | KTHNGKLCNL |
| 3982-5/A1 | ---------- | --XVKSDQIC | IGYHANNSTE | QVDTIMEKNV | TVTHAQDILE | KTHNGKLCNL |
| 3982-7/A1 | ---------- | --XVKSDQIC | IGYHANNSTE | QVDTIMEKNV | TVTHAQDILE | KTHNGKLCNL |
| 3982-8/A1 | --------XI | VSIVKSDQIC | IGYHANNSTE | QVDTIMEKNV | TVTHAQDILE | KTHNGKLCNL |
| 3982-9/A1 | ---------- | --XVKSDQIC | IGYHANNSTE | QVDTIMEKNV | TVTHAQDILE | KTHNGKLCNL |
| 3982-12/A1 | ---------- | XSIVKSDQIC | IGYHANNSTE | QVDTIMEKNV | TVTHAQDILE | KTHNGKLCNL |
| 3982-20/A1 | --------XI | VSIVKSDQIC | IGYHANNSTE | QVDTIMEKNV | TVTHAQDILE | KTHNGKLCNL |
| 3982-44/A1 | ---------- | --XVKSDQIC | IGYHANNSTE | QVDTIMEKNV | TVTHAQDILE | KTHNGKLCNL |
| 1553-2/B1 | MEKIVLLLAI | VSLVKSDQIC | IGYHANNSTE | QVDTIMEKNV | TVTHAQDILE | KTHNGKLCDL |
| 1553-6/B1 | MEKIVLLLAI | VSLVKSDQIC | IGYHANNSTE | QVDTIMEKNV | TVTHAQDILE | KTHNGKLCDL |
| 1553-13/B2 | MEKIVLLLAI | VSLVKSDQIC | IGYHANNSTE | QVDTIMEKNV | TVTHAQDILE | KTHNGKLCDL |
| 1553-26/B2 | MEKIVLLLAI | VSLVKSDQIC | IGYHANNSTE | QVDTIMEKNV | TVTHAQDILE | KTHNGKLCDL |
| 1553-28/B1 | MEKIVLLLAI | VSLVKSDQIC | IGYHANNSTE | QVDTIMEKNV | TVTHAQDILE | KTHNGKLCDL |
| 2095-39/B2 | MEKIVLLLAI | VSLVKSDQIC | IGYHANNSTE | QVDTIMEKNV | TVTHAQDILE | KTHNGKLCDL |
| 2095-46/B1 | -----XLLAI | VSLVKSDQIC | IGYHANNSTE | QVDTIMEKNV | TVTHAQDILE | KTHNGKLCDL |
| 2095-49/B1 | ----VLLLAI | VSLVKSDQIC | IGYHANNSTE | QVDTIMEKNV | TVTHAQDILE | KTHNGKLCDL |
| 2095-65/B1 | ---XVLLLAI | ISLVKSDQIC | IGYHANNSTE | QVDTIMEKNV | TVTHAQDILE | KTHNGKLCDL |
| 2095-68/B2 | ---IVLLLAI | VSLVKSDQIC | IGYHANNSTE | QVDTIMEKNV | TVTHAQDILE | KTHNGKLCDL |
| 2095-70/B2 | ------LLAI | VSLVKSDQIC | IGYHANNSTE | QVDTIMEKNV | TVTHAQDILE | KTHNGKLCDL |
| 2095-73/B2 | ---IVLLLAI | VSLVKSDQIC | IGYHANNSTE | QVDTIMEKNV | TVTHAQDILE | KTHNGKLCDL |
| 2095-75/B2 | ---IVLLLAI | VSLVKSDQIC | IGYHANNSTE | QVDTIMEKNV | TVTHAQDILE | KTHNGKLCDL |
| 3982-3/B1 | ----XLLLAI | VSLVKSDQIC | IGYHANNSTE | QVDTIMEKNV | TVTHAQDILE | KTHNGKLCDL |
| 3982-4/B1 | ---------- | --XVKSDQIC | IGYHANNSTE | QVDTIMEKNV | TVTHAQDILE | KTHNGKLCDL |
| 3982-13/B1 | ---------- | ----XSDQIC | IGYHANNSTE | QVDTIMEKNV | TVTHAQDILE | KTHNGKLCDL |
| 3982-14/B2 | ---------- | ----KGDQIC | IGYHANNSTE | QVDTIMEKNV | TVTHAQDILE | KTHNGKLCDL |
| 3982-19/B3 | ---------- | ---------- | --XHANNSTE | QVDTIMEKNI | TVTHAQDILE | KTHNGKLCDL |
| 3982-21/B2 | ---------- | -XLVKSDQIC | IGYHANNSTE | QVDTIMEKNV | TVTHAQDILE | KTHNGKLCDL |
| 3982-43/B1 | ---------- | ----KSDQIC | IGYHANNSTE | QVDTIMEKNV | TVTHAQDILE | KTHNGKLCDL |
| 3982-50/B1 | ---------- | --XVKSDQIC | IGYHANNSTE | QVDTIMEKNV | TVTHAQDILE | KTHNGKLCDL |
| 3982-52/B1 | ---------- | -XLVKSDQIC | IGYHANNSTE | QVDTIMEKNV | TVTHAQDILE | KTHNGKLCDL |
| 3982-55/A1 | ---------- | --XVKSDQIC | IGYHANNSTE | QVDTIMEKNV | TVTHAQDILE | KTHNGKLCNL |
| 3982-56/A1 | ---------- | ---------- | --XHANNSTE | QVDTIMEKNV | TVTHAQDILE | KTHNGKLCNL |
| 3982-78/B2 | ---------- | ---------- | ---HANNSTE | QVDTIMEKNV | TVTHAQDILE | KTHNGKLCDL |
| 4794-17/B | ------LLAI | VSLVKSDQIC | IGYHANNSTE | QVDTIMEKNV | TVTHAQDILE | KTHNGKLCDL |
| 4794-18/B | ------XLAI | VSLVKSDQIC | IGYHANNSTE | QVDTIMEKNV | TVTHAQDILE | KTHNGKLCDL |
| | ....\|....\| | ....\|....\| | ....\|....\| | ....\|....\| | ....\|....\| | ....\|....\| |
| | 65 | 75 | 85 | 95 | 105 | 115 |
| 1553-1/A1 | DGVKPLILRD | CSVAGWLLGN | PMCDEFLNVP | EWSYIVEKIN | PANDLCYPGK | FNDYEELKHL |
| 1553-15/A1 | DGVKPLILRD | CSVAGWLLGN | PMCDEFLNVP | EWSYIVEKIN | PANDLCYPGN | FNDYEELKHL |
| 2095-50/A1 | DGVKPLILRD | CSVAGWLLGN | PMCDEFLNVP | EWSYIVEKIY | PANDLCYPGN | FNDYEELKHL |
| 3982-2/A1 | DGVKPLILRD | CSVAGWLLGN | PMCDEFLDVP | EWSYIVEKIN | PANDLCYPGN | FNDYEELKHL |
| 3982-5/A1 | DGVKPLILRD | CSVAGWLLGN | PMCDEFLNV- | EWSYIVEKIN | PTNDLCYPGN | FNDYEELKHL |
| 3982-7/A1 | DGVKPLILRD | CSVAGWLLGN | PMCDKFLNVP | EWSYIVEKIN | PTNDLCYPGN | FNDYEELKHL |
| 3982-8/A1 | DGVKPLILRD | CSVAGWLLGN | PMCDEFLNVP | EWSYIVEKIN | PTNDLCYPGN | FNDYEELKHL |
| 3982-9/A1 | DGVKPLILRD | CSVAGWLLGN | PMCDEFLNVP | EWSYIVEKIN | PANDLCYPGN | FNDYEELKHL |
| 3982-12/A1 | DGVKPLILRD | CSVAGWLLGN | PMCDEFLNVP | EWSYIVEKIN | PANDLCYPGN | FNDYEELKHL |
| 3982-20/A1 | DGVKPLILRD | CSVAGWLLGN | PMCDEFLNVP | EWSYIVEKIN | PANDLCYPGN | FNDYEELKHL |
| 3982-44/A1 | DGVKPLILRD | CSVAGWLLGN | PMCDEFLNVP | EWSYIVEKIN | PANDLCYPGN | FNDYEELKHL |
| 1553-2/B1 | GGVKPLILRD | CSVAGWLLGN | PMCDEFPNVS | EWSYIVEKIN | PANDLCYPGN | FNNYEELKHL |
| 1553-6/B1 | DGVKPLILRD | CSVAGWLLGN | PMCDEFPNVS | EWSYIVEKTN | PANDLCYPGN | FNNYEELKHL |
| 1553-13/B2 | DGVKPLILRD | CSVAGWLLGN | PMCDEFPNVS | EWSYIVEKIN | PANDLCYPGN | FNNYEELKHL |
| 1553-26/B2 | DGVKPLILRD | CSVAGWLLGN | PMCDEFPNVS | EWSYIVEKIN | PANDLCYPGN | FNNYEELKHL |
| 1553-28/B1 | DGVKPLILRD | CSVAGWLLGN | PMCDEFPNVS | EWSYIVEKIN | PANDLCYPGN | FNNYEELKHL |
| 2095-39/B2 | DGVKPLILRD | CSVAGWLLGN | PMCDEFPNVS | EWSYIVEKTN | PANDLCYPGN | FNNYEELKHL |
| 2095-46/B1 | DGVKPLILRD | CSVAGWLLGN | PMCDEFPNVS | EWSYIVEKIN | PANDLCYPGN | FNNYEELKHL |
| 2095-49/B1 | DGVKPLILRD | CSVAGWLLGN | PMCDEFPNVS | EWSYIVEKIN | PANDLCYPGN | FNNYEELKHL |
| 2095-65/B1 | DGVKPLILRG | CSVAGWLLGN | PMCDEFPNVS | EWSYIVEKIN | PANDLCYPGN | FNNYEELKHL |
| 2095-68/B2 | DGVKPLILRD | CSVAGWLLGN | PMCDEFPNVS | EWSYIVEKIN | PANDLCYPGN | FNNYEELKHL |
| 2095-70/B2 | DGVKPLILRD | CSVAGWLLGN | PMCDEFPNVS | EWSYIVEKIN | PANDLCYPGN | FNNYEELKHL |
| 2095-73/B2 | DGVKPLILRD | CSVAGWLLGN | PMCDEFPNVS | EWSYIVEKIN | PANDLCYPGN | FNNYEELKHL |
| 2095-75/B2 | DGVKPLILRD | CSVAGWLLGN | PMCDEFPNVS | EWSYIVEKIN | PANDLCYPGN | FNNYEELKHL |
| 3982-3/B1 | DGVKPLILRD | CSVAGWLLGN | PMCDEFPNVS | EWSYIVEKIN | PANDLCYPGN | FNNYEELKHL |
| 3982-4/B1 | DGVKPLILRD | CSVAGWLLGN | PMCDEFPNVS | EWSYIVEKIN | PANDLCYPGN | FNNYEELKHL |
| 3982-13/B1 | DGVKPLILRD | CSVAGWLLGN | PMCDEFPNVS | EWSYIVEKIN | PANDLCYPGN | FNNYEELKHL |
| 3982-14/B2 | DGVKPLILRD | CSVAGWLLGN | PMCDEFPNVS | EWSYIVEKIN | PANDLCYPGN | FNNYEELKHL |
| 3982-19/B3 | DGVKPLILRD | CSVAGWLLGN | PMCDEFLNVS | EWSYIVEKIN | PANDLCYPGN | FNNYEELKHL |
| 3982-21/B2 | DGVKPLILRD | CSVAGWLLGN | PMCDEFPNVS | EWSYIVEKIN | PANDLCYPGN | FNNYEELKHL |
| 3982-43/B1 | DGVKPLILRD | CSVAGWLLGN | PMCDEFPNVS | EWSYIVEKIN | PANDLCYPGN | FNNYEELKHL |
| 3982-50/B1 | DGVKPLILRD | CSVAGWLLGN | PMCDEFPNVS | EWSYIVEKTN | PANDLCYPGN | FNNYEELKHL |
| 3982-52/B1 | DGVKPLILRD | CSVAGWLLGN | PMCDEFPNVS | EWSYIVEKIN | PANDLCYPGN | FNNYEELKHL |
| 3982-55/A1 | DGVKPLILRD | CSVAGWLLGN | PMCDKFLNVP | EWSYIVEKIN | PANDLCYPGD | FNDYEELKHL |
| 3982-56/A1 | DGVKPLILRD | CSVAGWLLGN | PMCDEFPNVL | EWSYIVEKIN | PANDLCYPGN | FNDYEELKHL |
| 3982-78/B2 | DGVKPLILRD | CSVAGWLLGN | PMCDEFPNVS | EWSYIVEKIN | PANDLCYPGN | FNNYEELKHL |
| 4794-17/B | DGVKPLILRD | CSVAGWLLGN | PMCDEFPNVS | EWSYIVEKIN | PANDLCYPGN | FNNYEELKHL |
| 4794-18/B | DGVKPLILRD | CSVAGWLLGN | PMCDEFPNVS | EWSYIVEKIN | PANDLCYPGN | FNNYEELKHL |
| | ....\|....\| | ....\|....\| | ....\|....\| | ....\|....\| | ....\|....\| | ....\|....\| |
| | 125 | 135 | 145 | 155 | 165 | 175 |
| 1553-1/A1 | LSRINHFEKI | QIIPRNSWSD | HETS-GVSSA | CQYQGRSSFF | RNVVWLTKKD | NAYSTIKRSY |
| 1553-15/A1 | LSRINHFEKI | QIIPKGSWSD | HEAS-GVSSA | CPYQGRSSFF | RNVVWLTKKN | NAYPTIKKSY |
| 2095-50/A1 | LSRINHFEKI | QIIPKSSWSD | HEAS-GVSSA | CPYQGRSSFF | RNVVWLTKKD | NAYPTIKKSY |
| 3982-2/A1 | LSRINHFEKI | QIIPKNSWSD | HETS-GVSSA | CPYQGRSSFF | RNVVWLTKKN | TAYPTIKKSY |
| 3982-5/A1 | LSRINHFEKI | QIIPKNYWSD | HETS-GVSSA | CPYQGRPSFF | RNVVWLTKKN | NAYPTIKKSY |
| 3982-7/A1 | LSRINHFEKI | QIIPKNSWSD | HEAS-GVSSA | CPYQGRSSFF | RNVVWLTKKN | NAYPTIKKSY |
| 3982-8/A1 | LSRINHFEKI | QIIPKNSWSD | HEAS-GVSSA | CPYQGRSSFF | RNVVWLTKKN | NAYPTIKKSY |
| 3982-9/A1 | LSRINHFEKI | QIIPKNSWSD | HEAS-GVSSA | CPYQGRSSFF | RNVVWLTKKN | NXYPTIKKSY |
| 3982-12/A1 | LSRINHFEKI | QIIPKNSWSD | HEAS-GVSSA | CPYQGRSSFF | RNVVWLTKKN | NAYPTIKKSY |
| 3982-20/A1 | LSRINHFEKI | QIIPKNSWSD | HEAS-GVSSA | CPYQGRSSFF | RNVVWLTKKN | NAYPTIKKSY |
| 3982-44/A1 | LSRINHFEKI | QIIPKNSWSD | HEAS-GVSSA | CPYQGRSSFF | RNVVWLTKKD | NAYPTIKRSY |
| 1553-2/B1 | LSRINRFEKI | QIIPKSSWPD | HEASLGVSSA | CPYQGGPSFY | RNVVWLIKKN | DTYPTIKESY |
| 1553-6/B1 | LSRINRFEKI | KIIPKSSWPD | HEASLGVSSA | CPYQGEPSFY | RNVVWLIKKN | NTYPTIKESY |
| 1553-13/B2 | LSRINRFEKI | KIIPKSSWPD | HEASLGVSSA | CPYQRGPSFY | RNVVWLIKKN | NTYPTIKKSY |
| 1553-26/B2 | LSRINRFEKI | KIIPKSSWPD | HEASLGVSSA | CPYQGGPSFY | RNVVWLIKKN | NTYPTIKESY |
| 1553-28/B1 | LSRINRFEKI | QIIPKSSWPD | HEASLGVSSA | CPYQGEPSFY | RNVVWLIKKN | NTYPTIKESY |
| 2095-39/B2 | LSRINRFEKI | KIIPKSSWPD | HEASLGVSSA | CPYQGRPSFY | RNVVWLIKKN | NTYPTIKESY |
| 2095-46/B1 | LSRINRFEKI | QIIPKSSWPD | HEASLGVSSA | CPYQGEPSFY | RNVVWLIKKN | NTYPTIKENY |
| 2095-49/B1 | LSRINRFEKI | QIIPKSSWPD | HEASLGVSSA | CPYQGEPSFY | RNVVWLIKKN | NTYPTIKENY |
| 2095-65/B1 | LSRINRFEKI | QIIPKSSWPD | HEASLGVSSA | CPYQGEPSFY | RNVVWLIKKN | NTYPTIKESY |
| 2095-68/B2 | LSRINRFEKI | KIIPKSSWPD | HEASLGVSSA | CPYQGGPSFY | RNVVWLIKKN | NTYPTIKKSY |
| 2095-70/B2 | LSRINRFEKI | KIIPKXSWPD | HEASLGVSSA | CPYQGGPSFY | RNVVWLIKKN | NTYPTIKESY |
| 2095-73/B2 | LSRINRFEKI | KIIPKSSWPD | HEASLGVSSA | CPYQEGPSFY | RNVVWLIKKN | NTYPTIKKSY |
| 2095-75/B2 | LSRINRFEKI | KIIPKSSWPD | HEASLGVSSA | CPYQGGXSFY | RNVVWLIKKN | NTYPTIKESY |
| 3982-3/B1 | LSRINRFEKI | QIIPKSSWPD | HEASLGVSSA | CPYQGEPSFY | RNVVWLIKKN | NTYPTIKENY |
| 3982-4/B1 | LSRINRFEKI | QIIPKSSWPD | HEASLGVSSA | CPYQGEPSFY | RNVVWLIKKN | NTYPTIKESY |
| 3982-13/B1 | LSRINRFEKI | QIIPKSSWPD | HEASLGVSSA | CPYQGEPSFY | RNVVWLIKKN | NTYPTIKENY |
| 3982-14/B2 | LSRINRFEKI | KIIPKSSWPD | HEASLGVSSA | CPYQGGPSFY | RNVVWLIKKN | NTYPTIKESY |
| 3982-19/B3 | LSRINRFEKI | QIISKNSWPD | HEASLGVSAA | CPYQGGLSFY | RNVVWLIEKN | NTYPLIKKNY |
| 3982-21/B2 | LSRINRFEKI | KIIPKSSWPD | HEASLGVSSA | CPYQRGPSFY | RNVVWLIKKN | NTYPTIKKSY |
| 3982-43/B1 | LSRINRFEKI | QIIPKSSWPD | HEASLGVSSA | CPYQGGPSFY | RNVVWLIKKN | DTYPTIKESY |
| 3982-50/B1 | LSRINRFEKI | KIIPKSSWPD | HEASLGVSSA | CPYQGEPSFY | RNVVWLIKKN | NTYPTIKESY |
| 3982-52/B1 | LSRINRFEKI | QIIPKSSWPD | HEASLGVSSA | CPYQGGPSFY | RNVVWLIKKN | DTYPTIKESY |
| 3982-55/A1 | LSRINHFEKI | QIIPKNSWSD | HEAS-GVSSA | CPYQGRSSFF | RNVVWLTKKN | NAYPTIKKSY |
| 3982-56/A1 | LSRINHFEKI | QIIPKNSWSD | HEAS-GVSSA | CPYQRRSSFF | RNVVWLTKKN | NAYPTIKKSY |
| 3982-78/B2 | LSRINRFEKI | KIIPKSSWPD | HEASLGVSSA | CPYQGGPSFY | RNVVWLIKKN | NTYPTIKKSY |
| 4794-17/B | LSRINRFEKI | KIIPKSSWPD | HEASLGVSSA | CPYQGGPSFY | RNVVWLIKKN | NTYPTIKESY |
| 4794-18/B | LSRINRFEKI | KIIPKSSWPD | HEASLGVSSA | CPYQGGPSFY | RNVVWLTKKN | NTYPTIKKSY |
| | ....\|....\| | ....\|....\| | ....\|....\| | ....\|....\| | ....\|....\| | ....\|....\| |
| | 185 | 195 | 205 | 215 | 225 | 235 |
| 1553-1/A1 | NNTNQEDLLV | LWGIHHPNDA | AEQTRLYQNP | TTYISVGTST | LNQRLVPKIA | TRSKVNGQSG |
| 1553-15/A1 | NNTNQEDLLV | LWGIHHPNDA | AEQTRLYQNP | TTYISVGTST | LNQRLVPKIA | TRSKVNGQSG |
| 2095-50/A1 | NNTNQEDLLI | LWGIHHPNDA | AEQTRLYQNP | TTYISVGTST | LNQRLVPKIA | TRSKVNGQSG |
| 3982-2/A1 | NNTNQEDLLV | LWGIHHPNDA | AEQTRLYQNP | TTYISVGTST | LNQRLVPKIA | TRSKVNGQSG |
| 3982-5/A1 | NNTNQEDLLV | LWGIHHPNDA | AEQTRLYQNP | TTYISVGTST | LNQRLVPKIA | TRSKVNGQSG |
| 3982-7/A1 | NNTNQEDLLV | LWGIHHPNDA | AEQTRLYQNP | TTYISVGTST | LNQRLVPKIA | TRSKVNGQSG |
| 3982-8/A1 | NNTNQEDLLV | LWGIHHPNDA | AEQTRLYQNP | TTYISVGTST | LNQRLVPKIA | TRSKVNGQSG |
| 3982-9/A1 | NNTNQEDLLV | LWGIHHPNDA | AEQTKLYQNP | TTYISVGTST | LNQRLVPKIA | TRSKVNGQSG |
| 3982-12/A1 | NNTNQEDLLV | LWGIHHPNDE | AEQTRLYQNP | TTYISVGTST | LNQRLVPKIA | TRSKVNGQSG |
| 3982-20/A1 | NNTNQEDLLV | LWGIHHPNDA | AEQTRLYQNP | TTYISVGTST | LNQRLVPKIA | NRSKVNGQSG |
| 3982-44/A1 | NNTNQEDLLV | LWGIHHPNDA | AEQTRLYQNP | TTYISVGTST | LNQRLVPKIA | TRSKVNGQSG |
| 1553-2/B1 | HNTNQEDLLV | LWGIHHPNNE | EEQKRIYKNP | TTYVSVGTST | LNQRLVPKIA | TRSKVNGQSG |
| 1553-6/B1 | HNTNQEDLLV | LWGIHHPNDE | EEQTRIYKNP | TTYISVGTST | LNQRLVPKIA | TRSKVNGQSG |
| 1553-13/B2 | HNTNQEDLLV | LWGIHHPNDE | EEQTRIYKNP | TTYISVGTST | LNQRLVPKIA | TRSKVNGQSG |
| 1553-26/B2 | HNTNQEDLLV | LWGIHHPNDE | EEQTRIYKNP | TTYISIGTST | LNQRLVPKIA | TRSKVNGQRG |
| 1553-28/B1 | HNTNQEDLLV | LWGIHHPNDE | EEQKRIYKNP | TTYISVGTST | LNQRLVPKIA | TRPKVNGQSG |
| 2095-39/B2 | HNTNQEDLLV | LWGIHHPNDE | EEQTRIYKNP | TTYISIGTST | LNQRLIPKIA | TRSKVNGQSG |
| 2095-46/B1 | HNTNQEDLLV | LWGIHHPNDE | EEQKRIYKNP | TTYISVGTST | LNQRLVPKIA | TRPKVNGQSG |
| 2095-49/B1 | HNTNQEDLLV | LWGIHHPNDE | EEQKRIYKNP | TTYISVGTST | LNQRLVPKIA | TRPKVNGQSG |
| 2095-65/B1 | HNTNQEDLLV | LWGIHHPNDE | EEQKRIYKNP | TTYISVGTST | LNQRLVPKIA | TRPKVNGQSG |
| 2095-68/B2 | HNTNQEDLLV | LWGIHHPNDE | EEQTRIYKNP | TTYISVGTST | LNQRLVPKIA | TRSKVNGQSG |
| 2095-70/B2 | HNTNQEDLLV | LWGIHHPNDE | EEQTRIYKNP | TTYISIGTST | LNQRLVPKIA | TRSKVNGQSG |
| 2095-73/B2 | HNTNQEDLLV | LWGIHHPNDE | EEQTRIYKNP | TTYISVGTST | LNQRLVPKIA | TRSKVNGQSG |
| 2095-75/B2 | HNTNQEDLLV | LWGIHHPNDE | EEQTRIYKNP | TTYISIGTST | LNQRLVPKIA | TRSKVNGQSG |
| 3982-3/B1 | HNTNQEDLLV | LWGIHHPNDE | EEQKRIYKNP | TTYISVGTST | LTQRLVPKIA | TRPKVNGQSG |
| 3982-4/B1 | HNTNQEDLLV | LWGIHHPNDE | EEQKRIYKNP | TTYISVGTST | LNQRLVPKIA | TRPKVNGQSG |
| 3982-13/B1 | HNTNQEDLLV | LWGIHHPNDE | EEQKRIYKNP | TTYISVGTST | LNQRLVPKIA | TRPKVNGQSG |
| 3982-14/B2 | HNTNQEDLLV | LWGIHHPNDE | EEQTRIYKNP | NTYISVGTST | LNQRLVPKIA | TRSKVNGQSG |
| 3982-19/B3 | HNTNQEDLLV | LWGIHHPNDE | AEQXRLYKNS | TTYISVGTST | LXQRLVPKIA | TRPKVNGQSG |
| 3982-21/B2 | HNTNQEDLLV | LWGIHHPNDE | EEQTRIYKNP | TTYISVGTST | LNQRLVPKIA | TRSKVNGQSG |
| 3982-43/B1 | HNTNQEDLLV | LWGIHHPNDE | EEQKRIYKNP | TTYISVGTST | LNQRLVPKIA | TRSKVNGQSG |
| 3982-50/B1 | HNTNQEDLLV | LWGIHHPNDE | EEQTRIYKNP | TTYISVGTST | LNQRLVPKIA | TRSKVNGQSG |
| 3982-52/B1 | HNTNQEDLLV | LWGIHHPNDE | EEQKRIYKNP | TTYVSVGTST | LNQRLVPKIA | TRSKVNGQSG |
| 3982-55/A1 | NNTNQEDLLV | LWGIHHPNDA | AEQTMLYQNP | TTYVSVGTST | LNQRLVPKIA | TRSKVNGQSG |
| 3982-56/A1 | NNTNQEDLLV | LWGIHHPNDA | AEQTRLYQNP | TTYISVGTST | LNQRLVPKIA | TRSKVNGQSG |
| 3982-78/B2 | HNTNQEDLLV | LWGIHHPNDE | EEQTRIYKNP | TTYISVGTST | LNQRLVPKIA | TRSKVNGQSG |
| 4794-17/B | HNINKEDLLV | LWGIHHPNDE | EEQIRIYKNP | TTYISVGTST | LNQRLVPKIA | TRSKVNGQSG |
| 4794-18/B | HNINKEDLLV | LWGIHHPNDE | EEQIRIYKNP | TTYISVGTST | LNQRLVPKIA | TRSKVNGQSG |
| | ....\|....\| | ....\|....\| | ....\|....\| | ....\|....\| | ....\|....\| | ....\|....\| |
| | 245 | 255 | 265 | 275 | 285 | 295 |
| 1553-1/A1 | RMEFFWTILK | SNDAINFESN | GNFIAPENAY | KIVKKGDSTI | MKSELEYGNC | NTKCQTPIGA |
| 1553-15/A1 | RMEFFWTILK | SNDAINFESN | GNFIAPENAY | KIVKKGDSTI | MKSELEYGNC | NTKCQTPIGA |
| 2095-50/A1 | RMEFFWTILK | SNDAINFESN | GNFIAPENAY | KIVKKGDSTI | MKSELEYGNC | NTKCQTPIGA |
| 3982-2/A1 | RMEFFWTILK | SNDAINFESN | GNFIAPENAY | KIVKKGDSTI | MKSELEYGNC | NTKCQTPIGA |
| 3982-5/A1 | RMEFFWTILK | SNDAINFESN | GNFIAPENAY | KIVKKGDSTI | MKSELEYGNC | NTKCQTPIGA |
| 3982-7/A1 | RMEFFWTILK | SNDAINFESN | GNFIAPENAY | KIVKKGDSTI | MKSELEYGDC | NTKCQTPIGA |
| 3982-8/A1 | RMEFFWTILK | SNDAINFESN | GNFIAPENAY | KIVKKGDSTI | MKSELEYGDC | NTKCQTPIGA |
| 3982-9/A1 | RMEFFWTILK | SNDAINFESN | GNFIAPENAY | KIVKKGDSTI | MKSELEYGNC | NTKCQTPIGA |
| 3982-12/A1 | RMEFFWTILK | SNDAINFESN | GNFIAPENAY | KIVKKGDSTI | MKSELEYGNC | NTKCQTPIGA |
| 3982-20/A1 | RMEFFWTILK | SNDAINFESN | GNFIAPENAY | KIVKKGDSTI | MKSELEYGNC | NTKCQTPIGA |
| 3982-44/A1 | RMEFFWTILK | SNDAINFESN | GNFIAPENAY | KIVKKGDSTI | MKSELEYGNC | NTKCQTPIGA |
| 1553-2/B1 | RVEFFWTILK | SNDTINFESN | GNFIAPENAY | KIVKKGDSTI | MKSELEYGNC | STKCQTPIGA |
| 1553-6/B1 | RVEFFWTILK | SNDTINFESN | GNFIAPENAY | KIVKKGDSTI | MKSELEYGNC | STKCQTPVGA |
| 1553-13/B2 | RVEFFWTILK | SNDTINFESN | GNFIAPENAY | KIVKKGDSTI | MKSELEYGNC | NTKCQTPIGA |
| 1553-26/B2 | RVEFFWTILK | SNDTINFESN | GNFIAPENAY | KIVKKGDSTI | MKSELEYGNC | NTKCQTPIGA |
| 1553-28/B1 | RVEFFWTILK | SNDTINFESN | GNFIAPKNAY | KIVKKGSSTI | MKSELEYGNC | STKCQTPIGA |
| 2095-39/B2 | RVEFFWTILK | SNDTINFESN | GNFIAPENAY | KIVKKGDSTI | MKSELEYGNC | NTKCQTPIGA |
| 2095-46/B1 | RVEFFWTILK | SNDTINFESN | GNFIAPKNAY | KIVKKGSSTI | MKSELEYGNC | STKCQTPIGA |
| 2095-49/B1 | RVEFFWTILK | SNDTINFESN | GNFIAPKNAY | KIVKKGSSTI | MKSELEYGNC | STKCQTPIGA |
| 2095-65/B1 | RVEFFWTILK | SNDTINFESN | GNFIAPKNAY | KIVKKGSSTI | MKSELEYGNC | STKCQTPIGA |
| 2095-68/B2 | RVEFFWTILK | SNDTINFESN | GNFIAPENAY | KIVKKGDSTI | MKSELEHGNC | NTKCQTPIGA |
| 2095-70/B2 | RVEFFWTILK | SNDTINFESN | GNFIAPENAY | KIVKKGDSTI | MKSELEYGNC | NTKCQTPIGA |
| 2095-73/B2 | RVEFFWTILK | SNDTINFESN | GNFIAPENAY | KIVKKGDSTI | MKSELEYGNC | NTKCQTPIGA |
| 2095-75/B2 | RVEFFWTILK | SNDTINFESN | GNFIAPENAY | KIVKKGDSTI | MKSELEYGNC | NTKCQTPIGA |
| 3982-3/B1 | RVEFFWTILK | SNDTINFESN | GNFIAPKNAY | KIVKKGSSTI | MKSELEYGNC | STKCQTPIGA |
| 3982-4/B1 | RVEFFWTILK | SNDTINFESN | GNFIAPKNAY | KIVKKGSSTI | MKSELEYGNC | STKCQTPIGA |
| 3982-13/B1 | RVEFFWTILK | SNDTINFESN | GNFIAPKNAY | KIVKKGSSTI | MKSELEYGNC | STKCQTPIGA |
| 3982-14/B2 | RVEFFWTILK | SNDTINFESN | GNFIAPENAY | KIVKKGDSTI | MKSELEYGNC | STKCQTPIGA |
| 3982-19/B3 | RVEFFWTILK | SNDVINFESN | GNFIAPENAY | KIVKKGDSTI | MKSDLEYGNC | STKCQTPIGA |
| 3982-21/B2 | RVEFFWTILK | SNDTINFESN | GNFIAPENAY | KIVKKGDSTI | MKSELEYGNC | NTKCQTPIGA |
| 3982-43/B1 | RVEFFWTILK | SNDTINFESN | GNFIAPENAY | KIVKKGDSTI | MKSELEYGNC | STKCQTPIGA |
| 3982-50/B1 | RVEFFWTILK | SNDTINFESN | GNFIAPENAY | KIVKKGDSTI | MKSELEYGNC | STKCQTPVGA |
| 3982-52/B1 | RVEFFWTILK | SNDTINFESN | GNFIAPENAY | KIVKKGDSTI | MKSELEYGNC | STKCQTPIGA |
| 3982-55/A1 | RMEFFWTILK | SNDAINFESN | GNFIAPENAY | KIVKKGDSTI | MKSELEYGNC | NTKCQTPIGA |
| 3982-56/A1 | RMEFFWTILK | SNDAINFESN | GNFIAPENAY | KIVKKGDSTI | MKSELEYGNC | NTKCQTPIGA |
| 3982-78/B2 | RVEFFWTILK | SNDTINFESN | GNFIAPENAY | KIVKKGDSTI | MKSELEYGNC | NTKCQTPIGA |
| 4794-17/B | RVEFFWTILK | SNDTINFESN | GNFIAPENAY | KIVKKGDSTI | MKSELEYGNC | NTKCQTPIGA |
| 4794-18/B | RVEFFWTILK | SNDTINFESN | GNFIAPENAY | KIVKKGDSTI | MKSELEYGNC | NTKCQTPIGA |
| | ....\|....\| | ....\|....\| | ....\|....\| | ....\|....\| | ....\|....\| | ....\|....\| |
| | 305 | 315 | 325 | 335 | 345 | 355 |
| 1553-1/A1 | INSSMPFHNI | HPLTIGECPK | YVKSNRLVLA | TGLRNSPQEE | RRRKKRGLFG | AIAGFIEGGW |
| 1553-15/A1 | INSSMPFHNI | HPLTIGECPK | YVKSNRLVLA | TGLRNSPQGE | RRRKKRGLFG | AIAGFIEGGW |
| 2095-50/A1 | INSSMPFHNI | HPLTIGECPK | YVKSNRLVLA | TGLRNSPQGE | RRRKKRGLFG | AIAGFIEGGW |
| 3982-2/A1 | INSSMPFHNI | HPLTIGECPK | YVKSNRLVLA | TGLRNSPHGE | RRRKKRGLFG | AIAGFIEGGW |
| 3982-5/A1 | INSSMPFHNI | HPLTIGECPK | YVKSNRLVLA | TGLRNSPQGE | RRRKKRGLFG | AIAGFIEGGW |
| 3982-7/A1 | INSSMPFHNI | HPLTIGECPK | YVKSNRLVLA | TGLRNSPQGE | RRRKKRGLFG | AIAGFIEGGW |
| 3982-8/A1 | INSSMPFHNI | HPLTIGECPK | YVKSNRLVLA | TGLRNSPQGE | RRRKKRGLFG | AIAGFIEGGW |
| 3982-9/A1 | INSSMPFHNI | HPLTIGECPK | YVKSNRLVLA | TGLRNSPQGE | RRRKKRGLFG | AIAGFIEGGW |
| 3982-12/A1 | INSSMPFHNI | HPLTIGECPK | YVKSNRLVLA | TGLRNSPQGE | RRRKKRGLFG | AIAGFIEGGW |
| 3982-20/A1 | INSSMPFHNI | HPLTIGECPK | YVKSNRLVLA | TGLRNSPQGE | RRRKKRGLFG | AIAGFIEGGW |
| 3982-44/A1 | INSSMPFHNI | HPLTIGECPK | YVKSNRLVLA | TGLRNSPQGE | RRRKKRGLFG | AIAGFIEGGW |
| 1553-2/B1 | INTSMPFHNI | HPLTIGECPK | YVKSNRLVLA | TGLRNSPQGE | GRRKKRGLFG | AIAGFIEGGW |
| 1553-6/B1 | INSSMPFHNI | HPLTIGECPK | YVKSNRLVLA | TGLRNSPQGE | GRRKKRGLFG | AIAGFIEGGW |
| 1553-13/B2 | INSSMPFHNI | HPLTIGECPK | YVKSNRLVLA | TGLRNSPQGE | GRRKKRGLFG | AIAGFIEGGW |
| 1553-26/B2 | INSSMPFHNI | HPLTIGECPK | YVKSNRLVLA | TGLRNSPQGE | GRRKKRGLFG | AIAGFIEGGW |
| 1553-28/B1 | INSSMPFHNI | HPLTIGECPK | YVKSNRLVLA | TGLRNSPQGE | GRRKKRGLFG | AIAGFIEGGW |
| 2095-39/B2 | INSSMPFHNI | HPLTIGECPK | YVKSNRLVLA | TGLRNSPQGE | GRRKKRGLFG | AIAGFIEGGW |
| 2095-46/B1 | INSSMPFHNI | HPLTIGECPK | YVKSNRLVLA | TGLRNSPQGE | GRRKKRGLFG | AIAGFIEGGW |
| 2095-49/B1 | INSSMPFHNI | HPLTIGECPK | YVKSNRLVLA | TGLRNSPQGE | GRRKKRGLFG | AIAGFIEGGW |
| 2095-65/B1 | INSSMPFHNI | HPLTIGECPK | YVKSNRLVLA | TGLRNSPQGE | GRRKKRGLFG | AIAGFIEGGW |
| 2095-68/B2 | INSSMPFHNI | HPLTIGECPK | YVKSNRLVLA | TGLRNSPQGE | GRRKKRGLFG | AIAGFIEGGW |
| 2095-70/B2 | INSSMPFHNI | HPLTIGECPK | YVKSNRLVLA | TGLRNSPQGE | GRRKKRGLFG | AIAGFIEGGW |
| 2095-73/B2 | INSSMPFHNI | HPLTIGECPK | YVKSNRLVLA | TGLRNSPQGE | GRRKKRGLFG | AIAGFIEGGW |
| 2095-75/B2 | INSSMPFHNI | HPLTIGECPK | YVKSNRLVLA | TXLRNSPQGE | GRRKKRGLFG | AIAGFIEGGW |
| 3982-3/B1 | INSSMPFHNI | HPLTIGECPK | YVKSNRLVLA | TGLRNSPQGE | GRRKKRGLFG | AIAGFIEGGW |
| 3982-4/B1 | INSSMPFHNI | HPLTIGECPK | YVKSNRLVLA | TGLRNSPQGE | GRRKKRGLFG | AIAGFIEGGW |
| 3982-13/B1 | INSSMPFHNI | HPLTIGECPK | YVKSNRLVLA | TGLRNSPQGE | GRRKKRGLFG | AIAGFIEGGW |
| 3982-14/B2 | INSSMPFHNI | HPLTIGECPK | YVKSNRLVLA | TGLRNSPQEE | GRRKKRGLFG | AIAGFIEGGW |
| 3982-19/B3 | INSSMPFHNI | HPLTIGECPK | YVKSNRLVLA | TGLRNSPQGE | RRRKKRGLFG | AIAGFIEGGW |
| 3982-21/B2 | INSSMPFHNI | HPLTIGECPK | YVKSNRLVLA | TGLRNSPQGE | GRRKKRGLFG | AIAGFIEGGW |
| 3982-43/B1 | INTSMPFHNI | HPLTIGECPK | YVKSNRLVLA | TGLRNSPQGE | GRRKKRGLFG | AIAGFIEGGW |
| 3982-50/B1 | INSSMPFHNI | HPLTIGECPK | YVXSNRLVLA | TGLRNSPQGE | GRRKKRGLFG | AIAGFIEGGW |
| 3982-52/B1 | INTSMPFHNI | HPLTIGECPK | YVKSNRLVLA | TGLRNSPQGE | GRRKKRGLFG | AIAGFIEGGW |
| 3982-55/A1 | INSSMPFHNI | HPLTIGECPK | YVKSNRLVLA | TGLRNSPQGE | RRRKKRGLFG | AIAGFIEGGW |
| 3982-56/A1 | INSSMPFHNI | HPLTIGECPK | YVKSNRLVLA | TGLRNSPQGE | RRRKKRGLFG | AIAGFIEGGW |
| 3982-78/B2 | INSSMPFHNI | HPLTIGECPK | YVKSNRLVLA | TGLRNSPQGE | GRRKKRGLFG | AIAGFIEGGW |
| 4794-17/B | INSSMPFHNI | HPLTIGECPK | YVKSNRLVLA | TGLRNSPQGE | GRRKKRGLFG | AIAGFIEGGW |
| 4794-18/B | INSSMPFHNI | HPLTIGECPK | YVKSNRLVLA | TGLRNSPQGE | GRRKKRGLFG | AIAGFIEGGW |
| | ....\|....\| | ....\|....\| | ....\|....\| | ....\|....\| | ....\|....\| | ....\|....\| |
| | 365 | 375 | 385 | 395 | 405 | 415 |
| 1553-1/A1 | QGMVDGWYGY | HHSNEQGSGY | AADKESTQKA | IDGVTNKVNS | IIDKMNTQFE | AVGREFNNLE |
| 1553-15/A1 | QGMVDGWYGY | HHSNEQGSGY | AADKESTQKA | IDGVTNKVNS | IIDKMNTQFE | AVGREFNNLE |
| 2095-50/A1 | QGMVDGWYGY | HHSNEQGSGY | AADKESTQKA | IDGVTNKVNS | IIDKMNTQFE | AVGREFNNLE |
| 3982-2/A1 | QGMVDGWYGY | HHSNEQGSGY | AADKESTQKA | IDGVTNKVNS | IIDKMNTQFE | AVGREFNNLE |
| 3982-5/A1 | QGMVDGWYGY | HHSNEQGSGY | AADKESTQKA | IDGVTNKVNS | IIDKMNTQFE | AVGREFNNLE |
| 3982-7/A1 | QGMVDGWYGY | HHSNEQGSGY | AADKESTQKA | IDGVTNKVNS | IIDKMNTQFE | AVGREFNNLE |
| 3982-8/A1 | QGMVDGWYGY | HHSNEQGSGY | AADKESTQKA | IDGVTNKVNS | IIDKMNTQFE | AVGREFNNLE |
| 3982-9/A1 | QGMVDGWYGY | HHSNEQGSGY | AADKESTQKA | IDGVTNKVNS | IIDKMNTQFE | AVGREFNNLE |
| 3982-12/A1 | QGMVDGWYGY | HHSNEQGSGY | AADKESTQKA | IDGVTNKVNS | IIDKMNTQFE | AVGREFNNLE |
| 3982-20/A1 | QGMVDGWYGY | HHSNEQGSGY | AADKESTQKA | IDGVTNKVNS | IIDKMNTQFE | AVGREFNNLE |
| 3982-44/A1 | QGMVDGWYGY | HHSNEQGSGY | AADKESTQKA | IDGVTNKVNS | IIDKMNTQFE | AVGREFNNLE |
| 1553-2/B1 | QGMVDGWYGY | HHSNEQGSGY | AADKESTQKA | IDGVTNKVNS | IIDKMNTQFE | AVGREFNNLE |
| 1553-6/B1 | QGMVDGWYGY | HHSNXQGSGY | AADRESTQKA | IDGVTNKVNS | IIDKMNTQFE | AVGREFNNLE |
| 1553-13/B2 | QGMVDGWYGY | HHSNEQGSGY | AADKESTQKA | IDGVTNKVNS | IIDKMNTQFE | AVGREFNNLE |
| 1553-26/B2 | QGMVDGWYGY | HHSNEQGSGY | AADKESTQKA | IDGVTNKVNS | IIDKMNTQFE | AVGREFNNLE |
| 1553-28/B1 | QGMVDGWYGY | HHSNEQGTGY | AADKESTQKA | IDGVTNKVNS | IIDKMNTQFE | AVGREFNNLE |
| 2095-39/B2 | QGMVDGWYGY | HHSNEQGSGY | AADKESTQKA | IDGVTNKVNS | IIDKMNTQFE | AVGREFNNLE |
| 2095-46/B1 | QGMVDGWYGY | HHSNEQGTGY | AADKESTQKA | IDGVTNKVNS | IIDKMNTQFE | AVGREFNNLE |
| 2095-49/B1 | QGMVDGWYGY | HHSNEQGTGY | AADKESTQKA | IDGVTNKVNS | IIDKMNTQFE | AVGREFNNLE |
| 2095-65/B1 | QGMVDGWYGY | HHSNEQGTGY | AADKESTQKA | IDGVTNKVNS | IIDKMNTQFE | AVGREFNNLE |
| 2095-68/B2 | QGMVDGWYGY | HHSNEQGSGY | AADKESTQKA | IDGVTNKVNS | IIDKMNTQFE | AVGREFNNLE |
| 2095-70/B2 | QGMVDGWYGY | HHSNEQGSGY | AADKESTQKA | IDGVTNKVNS | IIDKMNTQFE | AVGREFNNLE |
| 2095-73/B2 | QGMVDGWYGY | HHSNEQGSGY | AADKESTQKA | IDGVTNKVNS | IIDKMNTQFE | AVGREFNNLE |
| 2095-75/B2 | QGMVDGWYGY | HHSNEQGSGY | AADKESTQKA | IDGVTNKVNS | IIDKMNTQFE | AVGREFNNLE |
| 3982-3/B1 | QGMVDGWYGY | HHSNEQGTGY | AADKESTQKA | IDGVTNKVNS | IIDKMNTQFE | AVGREFNNLE |
| 3982-4/B1 | QGMVDGWYGY | HHSNEQGTGY | AADKESTQKA | IDGVTNKVNS | IIDKMNTQFE | AVGREFNNLE |
| 3982-13/B1 | QGMVDGWYGY | HHSNEQGTGY | AADKESTQKA | IDGVTNKVNS | IIDKMNTQFE | AVGREFNNLE |
| 3982-14/B2 | QGMVDGWYGY | HHSNEQGSGY | AADKESTQKA | IDGVTNKVNS | IIDKMNTQFE | AVGREFNNLE |
| 3982-19/B3 | QGMVDGWYGY | HHSNEQGSGY | AADKESTQKA | IDGVTNKVNS | IIDKMNTQFE | AVGREFNNLE |
| 3982-21/B2 | QGMVDGWYGY | HHSNEQGSGY | AADKESTQKA | IDGVTNKVNS | IIDKMNTQFE | AVGREFNNLE |
| 3982-43/B1 | QGMVDGWYGY | HHSNEQGSGY | AADKESTQKA | INGVTNKVNS | IIDKMNTQFE | AVGREFNNLE |
| 3982-50/B1 | QGMVDGWYGY | HHSNEQGSGY | AADRESTQKA | IDGVTNKVNS | IIDKMNTQFE | AVGREFNNLE |
| 3982-52/B1 | QGMVDGWYGY | HHSNEQGSGY | AADKESTQKA | IDGVTNKVNS | IIDKMNTQFE | AVGREFNNLE |
| 3982-55/A1 | QGMVDGWYGY | HHSNEQGSGY | AADKESTQKA | IDGVTNKVNS | IIDKMNTQFE | AVGREFNNLE |
| 3982-56/A1 | QGMVDGWYGY | HHSNEQGSGY | AADKESTQKA | IDGVTNKVNS | IIDKMNTQFE | AVGREFNNLE |
| 3982-78/B2 | QGMVDGWYGY | HHSNEQGSGY | AADKESTQKA | IDGVTNKVNS | IIDKMNTQFE | AVGREFNNLE |
| 4794-17/B | QGMVDGWYGY | HHSNEQGSGY | AADKESTQKA | IDGVTNKVNS | IIDKMNTQFE | AVGREFNNLE |
| 4794-18/B | QGMVDGWYGY | HHSNEQGSGY | AADKESTQKA | IDGVTNKVNS | IIDKMNTQFE | AVGREFNNLE |
| | ....\|....\| | ....\|....\| | ....\|....\| | ....\|....\| | ....\|....\| | ....\|....\| |
| | 425 | 435 | 445 | 455 | 465 | 475 |
| 1553-1/A1 | RRIENLNKKM | EDGFLDVWTY | NAELLVLMEN | ERTLDFHDSN | VKNLYDKVRL | QLRDNAKELG |
| 1553-15/A1 | RRIENLNKKM | EDGFLDVWTY | NAELLVLMEN | ERTLDFHDSN | VKNLYDRVRL | QLRDNAKELG |
| 2095-50/A1 | RRIENLNKKM | EDGFLDVWTY | NAELLVLMEN | ERTLDFHDSN | VKNLYDKVRL | QLRDNAKELG |
| 3982-2/A1 | RRIENLNKKM | EDGFLDVWTY | NAELLVLMEN | ERTLDFHDSN | VKNLYDKVRL | QLRDNAKELG |
| 3982-5/A1 | RRIENLNKKM | EDGFLDVWTY | NAELLVLMEN | ERTLDFHDSN | VKNLYDKVRL | QLRDNAKELG |
| 3982-7/A1 | RRIENLNKKM | EDGFLDVWTY | NAELLVLMEN | ERTLDFHDSN | VKNLYDKVRL | QLRDNAKELG |
| 3982-8/A1 | RRIENLNKKM | EDGFLDVWTY | NAELLVLMEN | ERTLDFHDSN | VKNLYDKVRL | QLRDNAKELG |
| 3982-9/A1 | RRIENLNKKM | EDGFLDVWTY | NAELLVLMEN | ERTLDFHDSN | VKNLYDKVRL | QLRDNAKELG |
| 3982-12/A1 | RRIENLNKKM | EDGFLDVWTY | NAELLVLMEN | ERTLDFHDSN | VKNLYDKVRL | QLRDNAKELG |
| 3982-20/A1 | RRIENLNKKM | EDGFLDVWTY | NAELLVLMEN | ERTLDFHDSN | VKNLYDKVRL | QLRDNAKELG |
| 3982-44/A1 | RRIENLNKKM | EDGFLDVWTY | NAELLVLMEN | ERTLDFHDSN | VKNLYDKVRL | QLRDNAKELG |
| 1553-2/B1 | KRIENLNKKM | EDGFLDVWTY | NAELLVLMEN | ERTLDFHDSN | VRNLYDKVRL | QLRDNAKELG |
| 1553-6/B1 | KRIENLNKKM | EDGFLDVWTY | NAELLVLMEN | ERTLDFHDSN | VKNLYDKVRL | QLRDNAKELG |
| 1553-13/B2 | KRIENLNKKM | EDGFLDVWTY | NAELLVLMEN | ERTLDFHDSN | VKNLYDKVRL | QLRDNAKELG |
| 1553-26/B2 | KRIENLNKKM | EDGFLDVWTY | NAELLVLMEN | ERTLDFHDSN | VKNLYDKVRL | QLRDNAKELG |
| 1553-28/B1 | KRIENLNKKM | EDGFLDVWTY | NAELLVLMEN | ERTLDFHDSN | VRNLYDKVRL | QLRDNAKELG |
| 2095-39/B2 | KRIENLNKKM | EDGFLDVWTY | NAELLVLMEN | ERTLDFHDSN | VKNLYDKVRL | QLRDNAKELG |
| 2095-46/B1 | KRIENLNKKM | EDGFLDVWTY | NAELLVLMEN | ERTLDFHDSN | VRNLYDKVRL | QLRDNAKELG |
| 2095-49/B1 | KRIENLNKKM | EDGFLDVWTY | NAELLVLMEN | ERTLDFHDSN | VRNLYDKVRL | QLRDNAKELG |
| 2095-65/B1 | KRIENLNKKM | EDGFLDVWTY | NAELLVLMEN | ERTLDFHDSN | VRNLYDKVRL | QLRDNAKELG |
| 2095-68/B2 | KRIENLNKKM | EDGFLDVWTY | NAELLVLMEN | ERTLDFHDSN | VKNLYDKVRL | QLRDNAKELG |
| 2095-70/B2 | KRIENLNKKM | EDGFLDVWTY | NAELLVLMEN | ERTLDFHDSN | VKNLYDKVRL | QLRDNAKELG |
| 2095-73/B2 | KRIENLNKKM | EDGFLDVWTY | NAELLVLMEN | ERTLDFHDSN | VKNLYDKVRL | QLRDNAKELG |
| 2095-75/B2 | KRIENLNKKM | EDGFLDVWTY | NAELLVLMEN | ERTLDFHDSN | VKNLYDKVRL | QLRDNAKELG |
| 3982-3/B1 | KRIENLNKKM | EDGFLDVWTY | NAELLVLMEN | ERTLDFHDSN | VRNLYDKVRL | QLRDNAKELG |
| 3982-4/B1 | KRIENLNKKM | EDGFLDVWTY | NAELLVLMEN | ERTLDFHDSN | VRNLYDKVRL | QLRDNAKELG |
| 3982-13/B1 | KRIENLNKKM | EDGFLDVWTY | NAELLVLMEN | ERTLDFHDSN | VRNLYDKVRL | QLRDNAKELG |
| 3982-14/B2 | KRIENLNKKM | EDGFLDVWTY | NAELLVLMEN | ERTLDFHDSN | VKNLYDKVRL | QLRDNAKELG |
| 3982-19/B3 | RRIENLNKKM | EDGFLDVWTY | NAELLVLMEN | ERTLDFHDSN | VKNLYDKVRL | QLRDNAKELG |
| 3982-21/B2 | KRIENLNKKM | EDGFLDVWTY | NAELLVLMEN | ERTLDFHDSN | VKNLYDKVRL | QLRDNAKELG |
| 3982-43/B1 | KRIENLNKKM | EDGFLDVWTY | NAELLVLMEN | ERTLDFHDSN | VRNLYDKVRL | QLRDNAKELG |
| 3982-50/B1 | KRIENLNKKM | EDGFLDVWTY | NAELLVLMEN | ERTLDFHDSN | VKNLYDKVRL | QLRDNAKELG |
| 3982-52/B1 | KRIENLNKKM | EDGFLDVWTY | NAELLVLMEN | ERTLDFHDSN | VRNLYDKVRL | QLRDNAKELG |
| 3982-55/A1 | RRIENLNKKM | EDGFLDVWTY | NAELLVLMEN | ERTLDFHDSN | VKNLYDKVRL | QLRDNAKELG |
| 3982-56/A1 | RRIENLNKKM | EDGFLDVWTY | NAELLVLMEN | ERTLDFHDSN | VKNLYDKVRL | QLRDNAKELG |
| 3982-78/B2 | KRIENLNKKM | EDGFLDVWTY | NAELLVLMEN | ERTLDFHDSN | VKNLYDKVRL | QLRDNAKELG |
| 4794-17/B | KRIENLNKKM | EDGFLDVWTY | NAELLVLMEN | ERTLDFHDSN | VKNLYDKVRL | QLRDNAKELG |
| 4794-18/B | KRIENLNKKM | EDGFLDVWTY | NAELLVLMEN | ERTLDFHDSN | VKNLYDKVRL | QLRDNAKELG |
| | ....\|....\| | ....\|....\| | ....\|....\| | ....\|....\| | ....\|....\| | ....\|....\| |
| | 485 | 495 | 505 | 515 | 525 | 535 |
| 1553-1/A1 | NGCFEFYHRC | DNECMESVRN | GTYDYPQYSE | EARLKREEIS | GVKLESIGTY | QILSIYSTVA |
| 1553-15/A1 | NGCFEFYHRC | DNECMESVRN | GTYDYPQYSE | EARLKREEIS | GVKLESIGTY | QILSIYSTVA |
| 2095-50/A1 | NGCFEFYHRC | DNECMESVRN | GTYDYPQYSE | EARLKREEIS | GVKLESIGTY | QILSIYSTVA |
| 3982-2/A1 | NGCFEFYHRC | DNECMESVRN | GTYDYPQYSE | EARLKREEIS | GVKLESIGTY | QILSIYSTVA |
| 3982-5/A1 | NGCFEFYHRC | DNECMESVRN | GTYDYPQYSE | EARLKREEIS | GVKLESIGTY | QILSIYSTVA |
| 3982-7/A1 | NGCFEFYHRC | DNECMESVRN | GTYDYPQYSE | EARLKREEIS | GVKLESIGTY | QILSIYSTVA |
| 3982-8/A1 | NGCFEFYHRC | DNECMESVRN | GTYDYPQYSE | EARLKREEIS | GVKLESIGTY | QILSIYSTVA |
| 3982-9/A1 | NGCFEFYHRC | DNECMESVRN | GTYDYPQYSE | EARLKREEIS | GVKLESIGTY | QIL------- |
| 3982-12/A1 | NGCFEFYHRC | DNECMESVRN | GTYDYPQYSE | EARLKREEIS | GVKLESIGTY | QILSIYSTVA |
| 3982-20/A1 | NGCFEFYHRC | DNECMESVRN | GTYDYPQYSE | EARLKREEIS | GVKLESIGTY | QILSIYX--- |
| 3982-44/A1 | NGCFEFYHRC | DNECMESVRN | GTYDYPQYSE | EARLKREEIS | GVKLESIGTY | QILSIYSTVA |
| 1553-2/B1 | NGCFEFYHRC | DNECMESVRN | GTYDYPQYSE | EARLKREEIS | GVKLESIGTY | QILSIYSTVA |
| 1553-6/B1 | NGCFEFYHRC | DNECIESVRN | GTYDYPQYSE | EARLKREEIS | GVKLESIGTY | QILSIYSTVA |
| 1553-13/B2 | NGCFEFYHRC | DNECMESVRN | GTYDYPQYSE | EARLKREEIS | GVKLESIGTY | QILSIYSTVA |
| 1553-26/B2 | NGCFEFYHRC | DNECMESVRN | GTYDYPQYSE | EARLKREEIS | GVKLESIGTY | QILSIYSTVA |
| 1553-28/B1 | NGCFEFYHKC | DNECMESVRN | GTYDYPQYSK | EARLKREEIS | GVKLESIGTY | QILSIYSTVT |
| 2095-39/B2 | NGCFEFYHRC | DNECMESVRN | GTYDYPQYSE | EARLKREEIS | GVKLESIGTY | QILSIYSTVA |
| 2095-46/B1 | NGCFEFYHKC | DNECMESVRN | GTYDYPQYSK | EARLKREEIS | GVKLESIGTY | QILSIYSTVA |
| 2095-49/B1 | NGCFEFYHKC | DNECMESVRN | GTYDYPQYSK | EARLKREEIS | GVKLESIGTY | QILSIYSTVA |
| 2095-65/B1 | NGCFEFYHKC | DNECMESVRN | GTYDYPQYSK | EARLKREEIS | GVKLESIGTY | QILSIYSTVA |
| 2095-68/B2 | NGCFEFYHRC | DNECMESVRN | GTYDYPQYSE | EARLNREEIS | GVKLESIGTY | QILSIYSTVA |
| 2095-70/B2 | NGCFEFYHRC | DNECMESVRN | GTYDYPQYSE | EARLKREEIS | GVKLESIGTY | QILSIYSTVA |
| 2095-73/B2 | NGCFEFYHRC | DNECMESVRN | GTYDYPQYSE | EARLKREEIS | GVKLESIGTY | QILSIYSTVA |
| 2095-75/B2 | NGCFEFYHRC | DNECMESVRN | GTYDYPQYSE | EARLKREEIS | GVKLESIGTY | QILSIYSTVA |
| 3982-3/B1 | NGCFEFYHKC | DNECMESVRN | GTYDYPQYSK | EARLKREEIS | GVKLESIGTY | QILSIYSTVA |
| 3982-4/B1 | NGCFEFYHKC | DNECMESVRN | GTYDYPQYSK | EARLKREEIS | GVKLESIGTY | QILSIYSTVA |
| 3982-13/B1 | NGCFEFYHKC | DNECMESVRN | GTYDYPQYSK | EARLKREEIS | GVKLESIGTY | QILSIYSTVA |
| 3982-14/B2 | NGCFEFYHRC | DNECMESVRN | GTYDYPQYSE | EARLKREEIS | GVKLESIGTY | QILSIYSTVA |
| 3982-19/B3 | NGCFEFYHRC | DNECMESVRN | GTYDYPQYSE | EARLKREEIS | GAKLESIGTY | QIX------- |
| 3982-21/B2 | NGCFEFYHRC | DNECMESVKN | GTYDYPQYSE | EARLKREEIS | GVKLESIGTY | QILSIYSTVA |
| 3982-43/B1 | NGCFEFYHRC | DNECMESVRN | GTYDYPQYSK | EARLKREEIS | GVKLESIGTY | QILSIYSTVA |
| 3982-50/B1 | NGCFEFYHRC | DNECIESVRN | GTYDYPQYSE | EARLKREEIS | GVKLESIGTY | QILSIYSTVA |
| 3982-52/B1 | NGCFEFYHRC | DNECMESVRN | GTYDYPQYSE | EARLKREEIS | GVKLEX---- | ---------- |
| 3982-55/A1 | NGCFEFYHRC | DNECMESVRN | GTYDYPQYSE | EARLKREEIS | GVKLESIGTX | QI-------- |
| 3982-56/A1 | NGCFEFYHRC | DNECMESVRN | GTYDYPQYSE | EARLKREEIS | GVKLESIGTX | QIX------- |
| 3982-78/B2 | NGCFEFYHRC | DNECMESVRN | GTYDYPQYSE | EARLKREEIS | GVKLESIGTY | QIL*IYSTVA |
| 4794-17/B | NGCFEFYHRC | DNECMESVRN | GTYDYPQYSE | EARLKREEIS | GVKLESIGTY | QILSIYSTVA |
| 4794-18/B | NGCFEFYHRC | DNECMESVRN | GTYDYPQYSE | EARLKREEIS | GVKLESIGTY | QILSIYSTVA |
| | ....\|....\| | ....\|....\| | ....\|.... | | | |
| | 545 | 555 | 565 | | | |
| 1553-1/A1 | SSLALAIMVA | GLFLWMCSNG | SLQCRICI* | | | |
| 1553-15/A1 | SSLALAIMVA | GLFLWMCSNG | SLQCRICI* | | | |
| 2095-50/A1 | SSLALAIMVA | GLFLWMCSNG | X-------- | | | |
| 3982-2/A1 | SSLALAIMVX | ---------- | --------- | | | |
| 3982-5/A1 | SSLALAIM-- | ---------- | --------- | | | |
| 3982-7/A1 | SSLALAIMVA | GLFLWMCSNG | X-------- | | | |
| 3982-8/A1 | SSLALAIMVX | ---------- | --------- | | | |
| 3982-9/A1 | ---------- | ---------- | --------- | | | |
| 3982-12/A1 | SSLALAIX-- | ---------- | --------- | | | |
| 3982-20/A1 | ---------- | ---------- | --------- | | | |
| 3982-44/A1 | SSLALAIX-- | ---------- | --------- | | | |
| 1553-2/B1 | SSLALAIMVA | GLFLWMCSNG | SLQCRICI* | | | |
| 1553-6/B1 | SSLALAIIVA | GLFLWMCSNG | SLQCRICI* | | | |
| 1553-13/B2 | SSLALAIMMA | GLFLWMCSNG | SLQCRICI* | | | |
| 1553-26/B2 | SSLALAIMMA | GLFLWMCSNG | SLQCRICI* | | | |
| 1553-28/B1 | SSLALAIMVA | GLSLWMCSNG | SLQCRICI* | | | |
| 2095-39/B2 | SSLALAIMMA | GLFLWX---- | --------- | | | |
| 2095-46/B1 | SSLALAIMVA | GLSLWMCSX- | --------- | | | |
| 2095-49/B1 | SSLALAIMVA | GLSLWMCSNG | SLQCRICI- | | | |
| 2095-65/B1 | SSLALAIMVA | GLSLWM---- | --------- | | | |
| 2095-68/B2 | SSLALAIMMA | GLFLWMCSNG | X-------- | | | |
| 2095-70/B2 | GSLALAIMMA | GLFLWMCSNG | SLQ------ | | | |
| 2095-73/B2 | SSLALAIMVX | ---------- | --------- | | | |
| 2095-75/B2 | GSLALAIMVX | ---------- | --------- | | | |
| 3982-3/B1 | SSLALAIMVA | GLSLWMCSNG | SLQ------ | | | |
| 3982-4/B1 | SSLALAIMVA | GLSLWMCSNG | SLQCRX--- | | | |
| 3982-13/B1 | SSLALAIMVA | X--------- | --------- | | | |
| 3982-14/B2 | SSLALAIMVA | GLFLWMCSNG | SLQCRICI* | | | |
| 3982-19/B3 | ---------- | ---------- | --------- | | | |
| 3982-21/B2 | SSLALAIM-- | ---------- | --------- | | | |
| 3982-43/B1 | SSLALAIM-- | ---------- | --------- | | | |
| 3982-50/B1 | SSLALAIM-- | ---------- | --------- | | | |
| 3982-52/B1 | ---------- | ---------- | --------- | | | |
| 3982-55/A1 | ---------- | ---------- | --------- | | | |
| 3982-56/A1 | ---------- | ---------- | --------- | | | |
| 3982-78/B2 | SSLALAX--- | ---------- | --------- | | | |
| 4794-17/B | SSLALAI--- | ---------- | --------- | | | |
| 4794-18/B | SSLALAIMMA | GLFLWMCSNG | X-------- | | | |

TABLE B
The following shows SEQ ID NOs: 62 to 99, which correspond to the
variant sequences as set forth in SEQ ID NOs: 9 to 46 starting with
the N-terminal amino acid D17, wherein the numbering of said first
N-terminal amino acid position (D17) refers to the amino acid
position 17 as exemplarily given in SEQ ID NO 8:

| ....\|....\| | ....\|....\| | ....\|....\| | ....\|....\| | ....\|....\| | ....\|....\| | |
|---|---|---|---|---|---|---|
| | 5 | 15 | 25 | 35 | 45 | 55 |
| 1553-1/A1 | (SEQ ID NO: 62) | DQIC | IGYHANNSTE | QVDTIMEKNV | TVTHAQDILE | KTHNGKLCNL |
| 1553-15/A1 | (SEQ ID NO: 63) | DQIC | IGYHANNSTE | QVDTIMEKNV | TVTHAQDILE | KTHNGKLCNL |
| 2095-50/A1 | (SEQ ID NO: 64) | DQIC | IGYHANNSTE | QVDTIMEKNV | TVTHAQDILE | KTHNGKLCNL |
| 3982-2/A1 | (SEQ ID NO: 65) | DQIC | IGYHANNSTE | QVDTIMEKNV | TVTHAQDILE | KTHNGKLCNL |
| 3982-5/A1 | (SEQ ID NO: 66) | DQIC | IGYHANNSTE | QVDTIMEKNV | TVTHAQDILE | KTHNGKLCNL |
| 3982-7/A1 | (SEQ ID NO: 67) | DQIC | IGYHANNSTE | QVDTIMEKNV | TVTHAQDILE | KTHNGKLCNL |
| 3982-8/A1 | (SEQ ID NO: 68) | DQIC | IGYHANNSTE | QVDTIMEKNV | TVTHAQDILE | KTHNGKLCNL |
| 3982-9/A1 | (SEQ ID NO: 69) | DQIC | IGYHANNSTE | QVDTIMEKNV | TVTHAQDILE | KTHNGKLCNL |
| 3982-12/A1 | (SEQ ID NO: 70) | DQIC | IGYHANNSTE | QVDTIMEKNV | TVTHAQDILE | KTHNGKLCNL |
| 3982-20/A1 | (SEQ ID NO: 71) | DQIC | IGYHANNSTE | QVDTIMEKNV | TVTHAQDILE | KTHNGKLCNL |
| 3982-44/A1 | (SEQ ID NO: 72) | DQIC | IGYHANNSTE | QVDTIMEKNV | TVTHAQDILE | KTHNGKLCNL |
| 1553-2/B1 | (SEQ ID NO: 73) | DQIC | IGYHANNSTE | QVDTIMEKNV | TVTHAQDILE | KTHNGKLCDL |
| 1553-6/B1 | (SEQ ID NO: 74) | DQIC | IGYHANNSTE | QVDTIMEKNV | TVTHAQDILE | KTHNGKLCDL |
| 1553-13/B2 | (SEQ ID NO: 75) | DQIC | IGYHANNSTE | QVDTIMEKNV | TVTHAQDILE | KTHNGKLCDL |
| 1553-26/B2 | (SEQ ID NO: 76) | DQIC | IGYHANNSTE | QVDTIMEKNV | TVTHAQDILE | KTHNGKLCDL |
| 1553-28/B1 | (SEQ ID NO: 77) | DQIC | IGYHANNSTE | QVDTIMEKNV | TVTHAQDILE | KTHNGKLCDL |
| 2095-39/B2 | (SEQ ID NO: 78) | DQIC | IGYHANNSTE | QVDTIMEKNV | TVTHAQDILE | KTHNGKLCDL |
| 2095-46/B1 | (SEQ ID NO: 79) | DQIC | IGYHANNSTE | QVDTIMEKNV | TVTHAQDILE | KTHNGKLCDL |
| 2095-49/B1 | (SEQ ID NO: 80) | DQIC | IGYHANNSTE | QVDTIMEKNV | TVTHAQDILE | KTHNGKLCDL |
| 2095-65/B1 | (SEQ ID NO: 81) | DQIC | IGYHANNSTE | QVDTIMEKNV | TVTHAQDILE | KTHNGKLCDL |
| 2095-68/B2 | (SEQ ID NO: 82) | DQIC | IGYHANNSTE | QVDTIMEKNV | TVTHAQDILE | KTHNGKLCDL |
| 2095-70/B2 | (SEQ ID NO: 83) | DQIC | IGYHANNSTE | QVDTIMEKNV | TVTHAQDILE | KTHNGKLCDL |
| 2095-73/B2 | (SEQ ID NO: 84) | DQIC | IGYHANNSTE | QVDTIMEKNV | TVTHAQDILE | KTHNGKLCDL |
| 2095-75/B2 | (SEQ ID NO: 85) | DQIC | IGYHANNSTE | QVDTIMEKNV | TVTHAQDILE | KTHNGKLCDL |
| 3982-3/B1 | (SEQ ID NO: 86) | DQIC | IGYHANNSTE | QVDTIMEKNV | TVTHAQDILE | KTHNGKLCDL |
| 3982-4/B1 | (SEQ ID NO: 87) | DQIC | IGYHANNSTE | QVDTIMEKNV | TVTHAQDILE | KTHNGKLCDL |
| 3982-13/B1 | (SEQ ID NO: 88) | DQIC | IGYHANNSTE | QVDTIMEKNV | TVTHAQDILE | KTHNGKLCDL |
| 3982-14/B2 | (SEQ ID NO: 89) | DQIC | IGYHANNSTE | QVDTIMEKNV | TVTHAQDILE | KTHNGKLCDL |
| 3982-19/B3 | (SEQ ID NO: 90) | DQIC | IGXHANNSTE | QVDTIMEKNI | TVTHAQDILE | KTHNGKLCDL |
| 3982-21/B2 | (SEQ ID NO: 91) | DQIC | IGYHANNSTE | QVDTIMEKNV | TVTHAQDILE | KTHNGKLCDL |
| 3982-43/B1 | (SEQ ID NO: 92) | DQIC | IGYHANNSTE | QVDTIMEKNV | TVTHAQDILE | KTHNGKLCDL |
| 3982-50/B1 | (SEQ ID NO: 93) | DQIC | IGYHANNSTE | QVDTIMEKNV | TVTHAQDILE | KTHNGKLCDL |
| 3982-52/B1 | (SEQ ID NO: 94) | DQIC | IGYHANNSTE | QVDTIMEKNV | TVTHAQDILE | KTHNGKLCDL |
| 3982-55/A1 | (SEQ ID NO: 95) | DQIC | IGYHANNSTE | QVDTIMEKNV | TVTHAQDILE | KTHNGKLCNL |
| 3982-56/A1 | (SEQ ID NO: 96) | DQIC | IGXHANNSTE | QVDTIMEKNV | TVTHAQDILE | KTHNGKLCNL |
| 3982-78/B2 | (SEQ ID NO: 97) | DQIC | IGYHANNSTE | QVDTIMEKNV | TVTHAQDILE | KTHNGKLCDL |
| 4794-17/B | (SEQ ID NO: 98) | DQIC | IGYHANNSTE | QVDTIMEKNV | TVTHAQDILE | KTHNGKLCDL |
| 4794-18/B | (SEQ ID NO: 99) | DQIC | IGYHANNSTE | QVDTIMEKNV | TVTHAQDILE | KTHNGKLCDL |
| | ....\|....\| | ....\|....\| | ....\|....\| | ....\|....\| | ....\|....\| | ....\|....\| |
| | 65 | 75 | 85 | 95 | 105 | 115 |
| 1553-1/A1 | DGVKPLILRD | CSVAGWLLGN | PMCDEFLNVP | EWSYIVEKIN | PANDLCYPGK | FNDYEELKHL |
| 1553-15/A1 | DGVKPLILRD | CSVAGWLLGN | PMCDEFLNVP | EWSYIVEKIN | PANDLCYPGN | FNDYEELKHL |
| 2095-50/A1 | DGVKPLILRD | CSVAGWLLGN | PMCDEFLNVP | EWSYIVEKIY | PANDLCYPGN | FNDYEELKHL |
| 3982-2/A1 | DGVKPLILRD | CSVAGWLLGN | PMCDEFLDVP | EWSYIVEKIN | PANDLCYPGN | FNDYEELKHL |
| 3982-5/A1 | DGVKPLILRD | CSVAGWLLGN | PMCDEFLNV- | EWSYIVEKIN | PTNDLCYPGN | FNDYEELKHL |
| 3982-7/A1 | DGVKPLILRD | CSVAGWLLGN | PMCDKFLNVP | EWSYIVEKIN | PTNDLCYPGN | FNDYEELKHL |
| 3982-8/A1 | DGVKPLILRD | CSVAGWLLGN | PMCDEFLNVP | EWSYIVEKIN | PTNDLCYPGN | FNDYEELKHL |
| 3982-9/A1 | DGVKPLILRD | CSVAGWLLGN | PMCDEFLNVP | EWSYIVEKIN | PANDLCYPGN | FNDYEELKHL |
| 3982-12/A1 | DGVKPLILRD | CSVAGWLLGN | PMCDEFLNVP | EWSYIVEKIN | PANDLCYPGN | FNDYEELKHL |
| 3982-20/A1 | DGVKPLILRD | CSVAGWLLGN | PMCDEFLNVP | EWSYIVEKIN | PANDLCYPGN | FNDYEELKHL |
| 3982-44/A1 | DGVKPLILRD | CSVAGWLLGN | PMCDEFLNVP | EWSYIVEKIN | PANDLCYPGN | FNDYEELKHL |
| 1553-2/B1 | GGVKPLILRD | CSVAGWLLGN | PMCDEFPNVS | EWSYIVEKIN | PANDLCYPGN | FNNYEELKHL |
| 1553-6/B1 | DGVKPLILRD | CSVAGWLLGN | PMCDEFPNVS | EWSYIVEKTN | PANDLCYPGN | FNNYEELKHL |
| 1553-13/B2 | DGVKPLILRD | CSVAGWLLGN | PMCDEFPNVS | EWSYIVEKIN | PANDLCYPGN | FNNYEELKHL |
| 1553-26/B2 | DGVKPLILRD | CSVAGWLLGN | PMCDEFPNVS | EWSYIVEKIN | PANDLCYPGN | FNNYEELKHL |
| 1553-28/B1 | DGVKPLILRD | CSVAGWLLGN | PMCDEFPNVS | EWSYIVEKIN | PANDLCYPGN | FNNYEELKHL |
| 2095-39/B2 | DGVKPLILRD | CSVAGWLLGN | PMCDEFPNVS | EWSYIVEKTN | PANDLCYPGN | FNNYEELKHL |
| 2095-46/B1 | DGVKPLILRD | CSVAGWLLGN | PMCDEFPNVS | EWSYIVEKIN | PANDLCYPGN | FNNYEELKHL |
| 2095-49/B1 | DGVKPLILRD | CSVAGWLLGN | PMCDEFPNVS | EWSYIVEKIN | PANDLCYPGN | FNNYEELKHL |
| 2095-65/B1 | DGVKPLILRG | CSVAGWLLGN | PMCDEFPNVS | EWSYIVEKIN | PANDLCYPGN | FNNYEELKHL |
| 2095-68/B2 | DGVKPLILRD | CSVAGWLLGN | PMCDEFPNVS | EWSYIVEKIN | PANDLCYPGN | FNNYEELKHL |
| 2095-70/B2 | DGVKPLILRD | CSVAGWLLGN | PMCDEFPNVS | EWSYIVEKIN | PANDLCYPGN | FNNYEELKHL |
| 2095-73/B2 | DGVKPLILRD | CSVAGWLLGN | PMCDEFPNVS | EWSYIVEKIN | PANDLCYPGN | FNNYEELKHL |
| 2095-75/B2 | DGVKPLILRD | CSVAGWLLGN | PMCDEFPNVS | EWSYIVEKIN | PANDLCYPGN | FNNYEELKHL |
| 3982-3/B1 | DGVKPLILRD | CSVAGWLLGN | PMCDEFPNVS | EWSYIVEKIN | PANDLCYPGN | FNNYEELKHL |
| 3982-4/B1 | DGVKPLILRD | CSVAGWLLGN | PMCDEFPNVS | EWSYIVEKIN | PANDLCYPGN | FNNYEELKHL |
| 3982-13/B1 | DGVKPLILRD | CSVAGWLLGN | PMCDEFPNVS | EWSYIVEKIN | PANDLCYPGN | FNNYEELKHL |
| 3982-14/B2 | DGVKPLILRD | CSVAGWLLGN | PMCDEFPNVS | EWSYIVEKIN | PANDLCYPGN | FNNYEELKHL |
| 3982-19/B3 | DGVKPLILRD | CSVAGWLLGN | PMCDEFLNVS | EWSYIVEKIN | PANDLCYPGN | FNNYEELKHL |
| 3982-21/B2 | DGVKPLILRD | CSVAGWLLGN | PMCDEFPNVS | EWSYIVEKIN | PANDLCYPGN | FNNYEELKHL |
| 3982-43/B1 | DGVKPLILRD | CSVAGWLLGN | PMCDEFPNVS | EWSYIVEKIN | PANDLCYPGN | FNNYEELKHL |
| 3982-50/B1 | DGVKPLILRD | CSVAGWLLGN | PMCDEFPNVS | EWSYIVEKTN | PANDLCYPGN | FNNYEELKHL |
| 3982-52/B1 | DGVKPLILRD | CSVAGWLLGN | PMCDEFPNVS | EWSYIVEKIN | PANDLCYPGN | FNNYEELKHL |
| 3982-55/A1 | DGVKPLILRD | CSVAGWLLGN | PMCDKFLNVP | EWSYIVEKIN | PANDLCYPGD | FNDYEELKHL |
| 3982-56/A1 | DGVKPLILRD | CSVAGWLLGN | PMCDEFPNVL | EWSYIVEKIN | PANDLCYPGN | FNDYEELKHL |
| 3982-78/B2 | DGVKPLILRD | CSVAGWLLGN | PMCDEFPNVS | EWSYIVEKIN | PANDLCYPGN | FNNYEELKHL |
| 4794-17/B | DGVKPLILRD | CSVAGWLLGN | PMCDEFPNVS | EWSYIVEKIN | PANDLCYPGN | FNNYEELKHL |
| 4794-18/B | DGVKPLILRD | CSVAGWLLGN | PMCDEFPNVS | EWSYIVEKIN | PANDLCYPGN | FNNYEELKHL |
| | ....\|....\| | ....\|....\| | ....\|....\| | ....\|....\| | ....\|....\| | ....\|....\| |
| | 125 | 135 | 145 | 155 | 165 | 175 |
| 1553-1/A1 | LSRINHFEKI | QIIPRNSWSD | HETS-GVSSA | CQYQGRSSFF | RNVVWLTKKD | NAYSTIKRSY |
| 1553-15/A1 | LSRINHFEKI | QIIPKGSWSD | HEAS-GVSSA | CPYQGRSSFF | RNVVWLTKKN | NAYPTIKKSY |
| 2095-50/A1 | LSRINHFEKI | QIIPKSSWSD | HEAS-GVSSA | CPYQGRSSFF | RNVVWLTKKD | NAYPTIKKSY |
| 3982-2/A1 | LSRINHFEKI | QIIPKNSWSD | HETS-GVSSA | CPYQGRSSFF | RNVVWLTKKN | TAYPTIKKSY |
| 3982-5/A1 | LSRINHFEKI | QIIPKNYWSD | HETS-GVSSA | CPYQGRPSFF | RNVVWLTKKN | NAYPTIKKSY |
| 3982-7/A1 | LSRINHFEKI | QIIPKNSWSD | HEAS-GVSSA | CPYQGRSSFF | RNVVWLTKKN | NAYPTIKKSY |
| 3982-8/A1 | LSRINHFEKI | QIIPKNSWSD | HEAS-GVSSA | CPYQGRSSFF | RNVVWLTKKN | NAYPTIKKSY |
| 3982-9/A1 | LSRINHFEKI | QIIPKNSWSD | HEAS-GVSSA | CPYQGRSSFF | RNVVWLTKKN | NXYPTIKKSY |
| 3982-12/A1 | LSRINHFEKI | QIIPKNSWSD | HEAS-GVSSA | CPYQGRSSFF | RNVVWLTKKN | NAYPTIKKSY |
| 3982-20/A1 | LSRINHFEKI | QIIPKNSWSD | HEAS-GVSSA | CPYQGRSSFF | RNVVWLTKKN | NAYPTIKKSY |
| 3982-44/A1 | LSRINHFEKI | QIIPKNSWSD | HEAS-GVSSA | CPYQGRSSFF | RNVVWLTKKD | NAYPTIKRSY |
| 1553-2/B1 | LSRINRFEKI | QIIPKSSWPD | HEASLGVSSA | CPYQGGPSFY | RNVVWLIKKN | DTYPTIKESY |
| 1553-6/B1 | LSRINRFEKI | KIIPKSSWPD | HEASLGVSSA | CPYQGEPSFY | RNVVWLIKKN | NTYPTIKESY |
| 1553-13/B2 | LSRINRFEKI | KIIPKSSWPD | HEASLGVSSA | CPYQRGPSFY | RNVVWLIKKN | NTYPTIKKSY |
| 1553-26/B2 | LSRINRFEKI | KIIPKSSWPD | HEASLGVSSA | CPYQGGPSFY | RNVVWLIKKN | NTYPTIKESY |
| 1553-28/B1 | LSRINRFEKI | QIIPKSSWPD | HEASLGVSSA | CPYQGEPSFY | RNVVWLIKKN | NTYPTIKESY |
| 2095-39/B2 | LSRINRFEKI | KIIPKSSWPD | HEASLGVSSA | CPYQGRPSFY | RNVVWLIKKN | NTYPTIKESY |
| 2095-46/B1 | LSRINRFEKI | QIIPKSSWPD | HEASLGVSSA | CPYQGEPSFY | RNVVWLIKKN | NTYPTIKENY |
| 2095-49/B1 | LSRINRFEKI | QIIPKSSWPD | HEASLGVSSA | CPYQGEPSFY | RNVVWLIKKN | NTYPTIKENY |
| 2095-65/B1 | LSRINRFEKI | QIIPKSSWPD | HEASLGVSSA | CPYQGEPSFY | RNVVWLIKKN | NTYPTIKESY |
| 2095-68/B2 | LSRINRFEKI | KIIPKSSWPD | HEASLGVSSA | CPYQGGPSFY | RNVVWLIKKN | NTYPTIKKSY |
| 2095-70/B2 | LSRINRFEKI | KIIPKXSWPD | HEASLGVSSA | CPYQGGPSFY | RNVVWLIKKN | NTYPTIKESY |
| 2095-73/B2 | LSRINRFEKI | KIIPKSSWPD | HEASLGVSSA | CPYQEGPSFY | RNVVWLIKKN | NTYPTIKKSY |
| 2095-75/B2 | LSRINRFEKI | KIIPKSSWPD | HEASLGVSSA | CPYQGGXSFY | RNVVWLIKKN | NTYPTIKESY |
| 3982-3/B1 | LSRINRFEKI | QIIPKSSWPD | HEASLGVSSA | CPYQGEPSFY | RNVVWLIKKN | NTYPTIKENY |
| 3982-4/B1 | LSRINRFEKI | QIIPKSSWPD | HEASLGVSSA | CPYQGEPSFY | RNVVWLIKKN | NTYPTIKESY |
| 3982-13/B1 | LSRINRFEKI | QIIPKSSWPD | HEASLGVSSA | CPYQGEPSFY | RNVVWLIKKN | NTYPTIKENY |
| 3982-14/B2 | LSRINRFEKI | KIIPKSSWPD | HEASLGVSSA | CPYQGGPSFY | RNVVWLIKKN | NTYPTIKESY |
| 3982-19/B3 | LSRINRFEKI | QIISKNSWPD | HEASLGVSAA | CPYQGGLSFY | RNVVWLIEKN | NTYPLIKKNY |
| 3982-21/B2 | LSRINRFEKI | KIIPKSSWPD | HEASLGVSSA | CPYQRGPSFY | RNVVWLIKKN | NTYPTIKKSY |
| 3982-43/B1 | LSRINRFEKI | QIIPKSSWPD | HEASLGVSSA | CPYQGGPSFY | RNVVWLIKKN | DTYPTIKESY |
| 3982-50/B1 | LSRINRFEKI | KIIPKSSWPD | HEASLGVSSA | CPYQGEPSFY | RNVVWLIKKN | NTYPTIKESY |
| 3982-52/B1 | LSRINRFEKI | QIIPKSSWPD | HEASLGVSSA | CPYQGGPSFY | RNVVWLIKKN | DTYPTIKESY |
| 3982-55/A1 | LSRINHFEKI | QIIPKNSWSD | HEAS-GVSSA | CPYQGRSSFF | RNVVWLTKKN | NAYPTIKKSY |
| 3982-56/A1 | LSRINHFEKI | QIIPKNSWSD | HEAS-GVSSA | CPYQRRSSFF | RNVVWLTKKN | NAYPTIKKSY |
| 3982-78/B2 | LSRINRFEKI | KIIPKSSWPD | HEASLGVSSA | CPYQGGPSFY | RNVVWLIKKN | NTYPTIKKSY |
| 4794-17/B | LSRINRFEKI | KIIPKSSWPD | HEASLGVSSA | CPYQGGPSFY | RNVVWLIKKN | NTYPTIKESY |
| 4794-18/B | LSRINRFEKI | KIIPKSSWPD | HEASLGVSSA | CPYQGGPSFY | RNVVWLTKKN | NTYPTIKKSY |
| | ....\|....\| | ....\|....\| | ....\|....\| | ....\|....\| | ....\|....\| | ....\|....\| |
| | 185 | 195 | 205 | 215 | 225 | 235 |
| 1553-1/A1 | NNTNQEDLLV | LWGIHHPNDA | AEQTRLYQNP | TTYISVGTST | LNQRLVPKIA | TRSKVNGQSG |
| 1553-15/A1 | NNTNQEDLLV | LWGIHHPNDA | AEQTRLYQNP | TTYISVGTST | LNQRLVPKIA | TRSKVNGQSG |
| 2095-50/A1 | NNTNQEDLLI | LWGIHHPNDA | AEQTRLYQNP | TTYISVGTST | LNQRLVPKIA | TRSKVNGQSG |
| 3982-2/A1 | NNTNQEDLLV | LWGIHHPNDA | AEQTRLYQNP | TTYISVGTST | LNQRLVPKIA | TRSKVNGQSG |
| 3982-5/A1 | NNTNQEDLLV | LWGIHHPNDA | AEQTRLYQNP | TTYISVGTST | LNQRLVPKIA | TRSKVNGQSG |
| 3982-7/A1 | NNTNQEDLLV | LWGIHHPNDA | AEQTRLYQNP | TTYISVGTST | LNQRLVPKIA | TRSKVNGQSG |
| 3982-8/A1 | NNTNQEDLLV | LWGIHHPNDA | AEQTRLYQNP | TTYISVGTST | LNQRLVPKIA | TRSKVNGQSG |
| 3982-9/A1 | NNTNQEDLLV | LWGIHHPNDA | AEQTKLYQNP | TTYISVGTST | LNQRLVPKIA | TRSKVNGQSG |
| 3982-12/A1 | NNTNQEDLLV | LWGIHHPNDE | AEQTRLYQNP | TTYISVGTST | LNQRLVPKIA | TRSKVNGQSG |
| 3982-20/A1 | NNTNQEDLLV | LWGIHHPNDA | AEQTRLYQNP | TTYISVGTST | LNQRLVPKIA | NRSKVNGQSG |
| 3982-44/A1 | NNTNQEDLLV | LWGIHHPNDA | AEQTRLYQNP | TTYISVGTST | LNQRLVPKIA | TRSKVNGQSG |
| 1553-2/B1 | HNTNQEDLLV | LWGIHHPNNE | EEQKRIYKNP | TTYVSVGTST | LNQRLVPKIA | TRSKVNGQSG |
| 1553-6/B1 | HNTNQEDLLV | LWGIHHPNDE | EEQTRIYKNP | TTYISVGTST | LNQRLVPKIA | TRSKVNGQSG |
| 1553-13/B2 | HNTNQEDLLV | LWGIHHPNDE | EEQTRIYKNP | TTYISVGTST | LNQRLVPKIA | TRSKVNGQSG |
| 1553-26/B2 | HNTNQEDLLV | LWGIHHPNDE | EEQTRIYKNP | TTYISIGTST | LNQRLVPKIA | TRSKVNGQRG |
| 1553-28/B1 | HNTNQEDLLV | LWGIHHPNDE | EEQKRIYKNP | TTYISVGTST | LNQRLVPKIA | TRPKVNGQSG |
| 2095-39/B2 | HNTNQEDLLV | LWGIHHPNDE | EEQTRIYKNP | TTYISIGTST | LNQRLIPKIA | TRSKVNGQSG |
| 2095-46/B1 | HNTNQEDLLV | LWGIHHPNDE | EEQKRIYKNP | TTYISVGTST | LNQRLVPKIA | TRPKVNGQSG |
| 2095-49/B1 | HNTNQEDLLV | LWGIHHPNDE | EEQKRIYKNP | TTYISVGTST | LNQRLVPKIA | TRPKVNGQSG |
| 2095-65/B1 | HNTNQEDLLV | LWGIHHPNDE | EEQKRIYKNP | TTYISVGTST | LNQRLVPKIA | TRPKVNGQSG |
| 2095-68/B2 | HNTNQEDLLV | LWGIHHPNDE | EEQTRIYKNP | TTYISVGTST | LNQRLVPKIA | TRSKVNGQSG |
| 2095-70/B2 | HNTNQEDLLV | LWGIHHPNDE | EEQTRIYKNP | TTYISIGTST | LNQRLVPKIA | TRSKVNGQSG |
| 2095-73/B2 | HNTNQEDLLV | LWGIHHPNDE | EEQTRIYKNP | TTYISVGTST | LNQRLVPKIA | TRSKVNGQSG |
| 2095-75/B2 | HNTNQEDLLV | LWGIHHPNDE | EEQTRIYKNP | TTYISIGTST | LNQRLVPKIA | TRSKVNGQSG |
| 3982-3/B1 | HNTNQEDLLV | LWGIHHPNDE | EEQKRIYKNP | TTYISVGTST | LTQRLVPKIA | TRPKVNGQSG |
| 3982-4/B1 | HNTNQEDLLV | LWGIHHPNDE | EEQKRIYKNP | TTYISVGTST | LNQRLVPKIA | TRPKVNGQSG |
| 3982-13/B1 | HNTNQEDLLV | LWGIHHPNDE | EEQKRIYKNP | TTYISVGTST | LNQRLVPKIA | TRPKVNGQSG |
| 3982-14/B2 | HNTNQEDLLV | LWGIHHPNDE | EEQTRIYKNP | NTYISVGTST | LNQRLVPKIA | TRSKVNGQSG |
| 3982-19/B3 | HNTNQEDLLV | LWGIHHPNDE | AEQXRLYKNS | TTYISVGTST | LXQRLVPKIA | TRPKVNGQSG |
| 3982-21/B2 | HNTNQEDLLV | LWGIHHPNDE | EEQTRIYKNP | TTYISVGTST | LNQRLVPKIA | TRSKVNGQSG |
| 3982-43/B1 | HNTNQEDLLV | LWGIHHPNDE | EEQKRIYKNP | TTYISVGTST | LNQRLVPKIA | TRSKVNGQSG |
| 3982-50/B1 | HNTNQEDLLV | LWGIHHPNDE | EEQTRIYKNP | TTYISVGTST | LNQRLVPKIA | TRSKVNGQSG |
| 3982-52/B1 | HNTNQEDLLV | LWGIHHPNDE | EEQKRIYKNP | TTYVSVGTST | LNQRLVPKIA | TRSKVNGQSG |
| 3982-55/A1 | NNTNQEDLLV | LWGIHHPNDA | AEQTMLYQNP | TTYVSVGTST | LNQRLVPKIA | TRSKVNGQSG |
| 3982-56/A1 | NNTNQEDLLV | LWGIHHPNDA | AEQTRLYQNP | TTYISVGTST | LNQRLVPKIA | TRSKVNGQSG |
| 3982-78/B2 | HNTNQEDLLV | LWGIHHPNDE | EEQTRIYKNP | TTYISVGTST | LNQRLVPKIA | TRSKVNGQSG |
| 4794-17/B | HNINKEDLLV | LWGIHHPNDE | EEQIRIYKNP | TTYISVGTST | LNQRLVPKIA | TRSKVNGQSG |
| 4794-18/B | HNINKEDLLV | LWGIHHPNDE | EEQIRIYKNP | TTYISVGTST | LNQRLVPKIA | TRSKVNGQSG |
| | ....\|....\| | ....\|....\| | ....\|....\| | ....\|....\| | ....\|....\| | ....\|....\| |
| | 245 | 255 | 265 | 275 | 285 | 295 |
| 1553-1/A1 | RMEFFWTILK | SNDAINFESN | GNFIAPENAY | KIVKKGDSTI | MKSELEYGNC | NTKCQTPIGA |
| 1553-15/A1 | RMEFFWTILK | SNDAINFESN | GNFIAPENAY | KIVKKGDSTI | MKSELEYGNC | NTKCQTPIGA |
| 2095-50/A1 | RMEFFWTILK | SNDAINFESN | GNFIAPENAY | KIVKKGDSTI | MKSELEYGNC | NTKCQTPIGA |
| 3982-2/A1 | RMEFFWTILK | SNDAINFESN | GNFIAPENAY | KIVKKGDSTI | MKSELEYGNC | NTKCQTPIGA |
| 3982-5/A1 | RMEFFWTILK | SNDAINFESN | GNFIAPENAY | KIVKKGDSTI | MKSELEYGNC | NTKCQTPIGA |
| 3982-7/A1 | RMEFFWTILK | SNDAINFESN | GNFIAPENAY | KIVKKGDSTI | MKSELEYGDC | NTKCQTPIGA |
| 3982-8/A1 | RMEFFWTILK | SNDAINFESN | GNFIAPENAY | KIVKKGDSTI | MKSELEYGDC | NTKCQTPIGA |
| 3982-9/A1 | RMEFFWTILK | SNDAINFESN | GNFIAPENAY | KIVKKGDSTI | MKSELEYGNC | NTKCQTPIGA |
| 3982-12/A1 | RMEFFWTILK | SNDAINFESN | GNFIAPENAY | KIVKKGDSTI | MKSELEYGNC | NTKCQTPIGA |
| 3982-20/A1 | RMEFFWTILK | SNDAINFESN | GNFIAPENAY | KIVKKGDSTI | MKSELEYGNC | NTKCQTPIGA |
| 3982-44/A1 | RMEFFWTILK | SNDAINFESN | GNFIAPENAY | KIVKKGDSTI | MKSELEYGNC | NTKCQTPIGA |
| 1553-2/B1 | RVEFFWTILK | SNDTINFESN | GNFIAPENAY | KIVKKGDSTI | MKSELEYGNC | STKCQTPIGA |
| 1553-6/B1 | RVEFFWTILK | SNDTINFESN | GNFIAPENAY | KIVKKGDSTI | MKSELEYGNC | STKCQTPVGA |
| 1553-13/B2 | RVEFFWTILK | SNDTINFESN | GNFIAPENAY | KIVKKGDSTI | MKSELEYGNC | NTKCQTPIGA |
| 1553-26/B2 | RVEFFWTILK | SNDTINFESN | GNFIAPENAY | KIVKKGDSTI | MKSELEYGNC | NTKCQTPIGA |
| 1553-28/B1 | RVEFFWTILK | SNDTINFESN | GNFIAPKNAY | KIVKKGSSTI | MKSELEYGNC | STKCQTPIGA |
| 2095-39/B2 | RVEFFWTILK | SNDTINFESN | GNFIAPENAY | KIVKKGDSTI | MKSELEYGNC | NTKCQTPIGA |
| 2095-46/B1 | RVEFFWTILK | SNDTINFESN | GNFIAPKNAY | KIVKKGSSTI | MKSELEYGNC | STKCQTPIGA |
| 2095-49/B1 | RVEFFWTILK | SNDTINFESN | GNFIAPKNAY | KIVKKGSSTI | MKSELEYGNC | STKCQTPIGA |
| 2095-65/B1 | RVEFFWTILK | SNDTINFESN | GNFIAPKNAY | KIVKKGSSTI | MKSELEYGNC | STKCQTPIGA |
| 2095-68/B2 | RVEFFWTILK | SNDTINFESN | GNFIAPENAY | KIVKKGDSTI | MKSELEHGNC | NTKCQTPIGA |
| 2095-70/B2 | RVEFFWTILK | SNDTINFESN | GNFIAPENAY | KIVKKGDSTI | MKSELEYGNC | NTKCQTPIGA |
| 2095-73/B2 | RVEFFWTILK | SNDTINFESN | GNFIAPENAY | KIVKKGDSTI | MKSELEYGNC | NTKCQTPIGA |
| 2095-75/B2 | RVEFFWTILK | SNDTINFESN | GNFIAPENAY | KIVKKGDSTI | MKSELEYGNC | NTKCQTPIGA |
| 3982-3/B1 | RVEFFWTILK | SNDTINFESN | GNFIAPKNAY | KIVKKGSSTI | MKSELEYGNC | STKCQTPIGA |
| 3982-4/B1 | RVEFFWTILK | SNDTINFESN | GNFIAPKNAY | KIVKKGSSTI | MKSELEYGNC | STKCQTPIGA |
| 3982-13/B1 | RVEFFWTILK | SNDTINFESN | GNFIAPKNAY | KIVKKGSSTI | MKSELEYGNC | STKCQTPIGA |
| 3982-14/B2 | RVEFFWTILK | SNDTINFESN | GNFIAPENAY | KIVKKGDSTI | MKSELEYGNC | STKCQTPIGA |
| 3982-19/B3 | RVEFFWTILK | SNDVINFESN | GNFIAPENAY | KIVKKGDSTI | MKSDLEYGNC | STKCQTPIGA |
| 3982-21/B2 | RVEFFWTILK | SNDTINFESN | GNFIAPENAY | KIVKKGDSTI | MKSELEYGNC | NTKCQTPIGA |
| 3982-43/B1 | RVEFFWTILK | SNDTINFESN | GNFIAPENAY | KIVKKGDSTI | MKSELEYGNC | STKCQTPIGA |
| 3982-50/B1 | RVEFFWTILK | SNDTINFESN | GNFIAPENAY | KIVKKGDSTI | MKSELEYGNC | STKCQTPVGA |
| 3982-52/B1 | RVEFFWTILK | SNDTINFESN | GNFIAPENAY | KIVKKGDSTI | MKSELEYGNC | STKCQTPIGA |
| 3982-55/A1 | RMEFFWTILK | SNDAINFESN | GNFIAPENAY | KIVKKGDSTI | MKSELEYGNC | NTKCQTPIGA |
| 3982-56/A1 | RMEFFWTILK | SNDAINFESN | GNFIAPENAY | KIVKKGDSTI | MKSELEYGNC | NTKCQTPIGA |
| 3982-78/B2 | RVEFFWTILK | SNDTINFESN | GNFIAPENAY | KIVKKGDSTI | MKSELEYGNC | NTKCQTPIGA |
| 4794-17/B | RVEFFWTILK | SNDTINFESN | GNFIAPENAY | KIVKKGDSTI | MKSELEYGNC | NTKCQTPIGA |
| 4794-18/B | RVEFFWTILK | SNDTINFESN | GNFIAPENAY | KIVKKGDSTI | MKSELEYGNC | NTKCQTPIGA |
| | ....\|....\| | ....\|....\| | ....\|....\| | ....\|....\| | ....\|....\| | ....\|....\| |
| | 305 | 315 | 325 | 335 | 345 | 355 |
| 1553-1/A1 | INSSMPFHNI | HPLTIGECPK | YVKSNRLVLA | TGLRNSPQEE | RRRKKRGLFG | AIAGFIEGGW |
| 1553-15/A1 | INSSMPFHNI | HPLTIGECPK | YVKSNRLVLA | TGLRNSPQGE | RRRKKRGLFG | AIAGFIEGGW |
| 2095-50/A1 | INSSMPFHNI | HPLTIGECPK | YVKSNRLVLA | TGLRNSPQGE | RRRKKRGLFG | AIAGFIEGGW |
| 3982-2/A1 | INSSMPFHNI | HPLTIGECPK | YVKSNRLVLA | TGLRNSPHGE | RRRKKRGLFG | AIAGFIEGGW |
| 3982-5/A1 | INSSMPFHNI | HPLTIGECPK | YVKSNRLVLA | TGLRNSPQGE | RRRKKRGLFG | AIAGFIEGGW |
| 3982-7/A1 | INSSMPFHNI | HPLTIGECPK | YVKSNRLVLA | TGLRNSPQGE | RRRKKRGLFG | AIAGFIEGGW |
| 3982-8/A1 | INSSMPFHNI | HPLTIGECPK | YVKSNRLVLA | TGLRNSPQGE | RRRKKRGLFG | AIAGFIEGGW |
| 3982-9/A1 | INSSMPFHNI | HPLTIGECPK | YVKSNRLVLA | TGLRNSPQGE | RRRKKRGLFG | AIAGFIEGGW |
| 3982-12/A1 | INSSMPFHNI | HPLTIGECPK | YVKSNRLVLA | TGLRNSPQGE | RRRKKRGLFG | AIAGFIEGGW |
| 3982-20/A1 | INSSMPFHNI | HPLTIGECPK | YVKSNRLVLA | TGLRNSPQGE | RRRKKRGLFG | AIAGFIEGGW |
| 3982-44/A1 | INSSMPFHNI | HPLTIGECPK | YVKSNRLVLA | TGLRNSPQGE | RRRKKRGLFG | AIAGFIEGGW |
| 1553-2/B1 | INTSMPFHNI | HPLTIGECPK | YVKSNRLVLA | TGLRNSPQGE | GRRKKRGLFG | AIAGFIEGGW |
| 1553-6/B1 | INSSMPFHNI | HPLTIGECPK | YVKSNRLVLA | TGLRNSPQGE | GRRKKRGLFG | AIAGFIEGGW |
| 1553-13/B2 | INSSMPFHNI | HPLTIGECPK | YVKSNRLVLA | TGLRNSPQGE | GRRKKRGLFG | AIAGFIEGGW |
| 1553-26/B2 | INSSMPFHNI | HPLTIGECPK | YVKSNRLVLA | TGLRNSPQGE | GRRKKRGLFG | AIAGFIEGGW |
| 1553-28/B1 | INSSMPFHNI | HPLTIGECPK | YVKSNRLVLA | TGLRNSPQGE | GRRKKRGLFG | AIAGFIEGGW |
| 2095-39/B2 | INSSMPFHNI | HPLTIGECPK | YVKSNRLVLA | TGLRNSPQGE | GRRKKRGLFG | AIAGFIEGGW |
| 2095-46/B1 | INSSMPFHNI | HPLTIGECPK | YVKSNRLVLA | TGLRNSPQGE | GRRKKRGLFG | AIAGFIEGGW |
| 2095-49/B1 | INSSMPFHNI | HPLTIGECPK | YVKSNRLVLA | TGLRNSPQGE | GRRKKRGLFG | AIAGFIEGGW |
| 2095-65/B1 | INSSMPFHNI | HPLTIGECPK | YVKSNRLVLA | TGLRNSPQGE | GRRKKRGLFG | AIAGFIEGGW |
| 2095-68/B2 | INSSMPFHNI | HPLTIGECPK | YVKSNRLVLA | TGLRNSPQGE | GRRKKRGLFG | AIAGFIEGGW |
| 2095-70/B2 | INSSMPFHNI | HPLTIGECPK | YVKSNRLVLA | TGLRNSPQGE | GRRKKRGLFG | AIAGFIEGGW |
| 2095-73/B2 | INSSMPFHNI | HPLTIGECPK | YVKSNRLVLA | TGLRNSPQGE | GRRKKRGLFG | AIAGFIEGGW |
| 2095-75/B2 | INSSMPFHNI | HPLTIGECPK | YVKSNRLVLA | TXLRNSPQGE | GRRKKRGLFG | AIAGFIEGGW |
| 3982-3/B1 | INSSMPFHNI | HPLTIGECPK | YVKSNRLVLA | TGLRNSPQGE | GRRKKRGLFG | AIAGFIEGGW |
| 3982-4/B1 | INSSMPFHNI | HPLTIGECPK | YVKSNRLVLA | TGLRNSPQGE | GRRKKRGLFG | AIAGFIEGGW |
| 3982-13/B1 | INSSMPFHNI | HPLTIGECPK | YVKSNRLVLA | TGLRNSPQGE | GRRKKRGLFG | AIAGFIEGGW |
| 3982-14/B2 | INSSMPFHNI | HPLTIGECPK | YVKSNRLVLA | TGLRNSPQEE | GRRKKRGLFG | AIAGFIEGGW |
| 3982-19/B3 | INSSMPFHNI | HPLTIGECPK | YVKSNRLVLA | TGLRNSPQGE | RRRKKRGLFG | AIAGFIEGGW |
| 3982-21/B2 | INSSMPFHNI | HPLTIGECPK | YVKSNRLVLA | TGLRNSPQGE | GRRKKRGLFG | AIAGFIEGGW |
| 3982-43/B1 | INTSMPFHNI | HPLTIGECPK | YVKSNRLVLA | TGLRNSPQGE | GRRKKRGLFG | AIAGFIEGGW |
| 3982-50/B1 | INSSMPFHNI | HPLTIGECPK | YVXSNRLVLA | TGLRNSPQGE | GRRKKRGLFG | AIAGFIEGGW |
| 3982-52/B1 | INTSMPFHNI | HPLTIGECPK | YVKSNRLVLA | TGLRNSPQGE | GRRKKRGLFG | AIAGFIEGGW |
| 3982-55/A1 | INSSMPFHNI | HPLTIGECPK | YVKSNRLVLA | TGLRNSPQGE | RRRKKRGLFG | AIAGFIEGGW |
| 3982-56/A1 | INSSMPFHNI | HPLTIGECPK | YVKSNRLVLA | TGLRNSPQGE | RRRKKRGLFG | AIAGFIEGGW |
| 3982-78/B2 | INSSMPFHNI | HPLTIGECPK | YVKSNRLVLA | TGLRNSPQGE | GRRKKRGLFG | AIAGFIEGGW |
| 4794-17/B | INSSMPFHNI | HPLTIGECPK | YVKSNRLVLA | TGLRNSPQGE | GRRKKRGLFG | AIAGFIEGGW |
| 4794-18/B | INSSMPFHNI | HPLTIGECPK | YVKSNRLVLA | TGLRNSPQGE | GRRKKRGLFG | AIAGFIEGGW |
| | ....\|....\| | ....\|....\| | ....\|....\| | ....\|....\| | ....\|....\| | ....\|....\| |
| | 365 | 375 | 385 | 395 | 405 | 415 |
| 1553-1/A1 | QGMVDGWYGY | HHSNEQGSGY | AADKESTQKA | IDGVTNKVNS | IIDKMNTQFE | AVGREFNNLE |
| 1553-15/A1 | QGMVDGWYGY | HHSNEQGSGY | AADKESTQKA | IDGVTNKVNS | IIDKMNTQFE | AVGREFNNLE |
| 2095-50/A1 | QGMVDGWYGY | HHSNEQGSGY | AADKESTQKA | IDGVTNKVNS | IIDKMNTQFE | AVGREFNNLE |
| 3982-2/A1 | QGMVDGWYGY | HHSNEQGSGY | AADKESTQKA | IDGVTNKVNS | IIDKMNTQFE | AVGREFNNLE |
| 3982-5/A1 | QGMVDGWYGY | HHSNEQGSGY | AADKESTQKA | IDGVTNKVNS | IIDKMNTQFE | AVGREFNNLE |
| 3982-7/A1 | QGMVDGWYGY | HHSNEQGSGY | AADKESTQKA | IDGVTNKVNS | IIDKMNTQFE | AVGREFNNLE |
| 3982-8/A1 | QGMVDGWYGY | HHSNEQGSGY | AADKESTQKA | IDGVTNKVNS | IIDKMNTQFE | AVGREFNNLE |
| 3982-9/A1 | QGMVDGWYGY | HHSNEQGSGY | AADKESTQKA | IDGVTNKVNS | IIDKMNTQFE | AVGREFNNLE |
| 3982-12/A1 | QGMVDGWYGY | HHSNEQGSGY | AADKESTQKA | IDGVTNKVNS | IIDKMNTQFE | AVGREFNNLE |
| 3982-20/A1 | QGMVDGWYGY | HHSNEQGSGY | AADKESTQKA | IDGVTNKVNS | IIDKMNTQFE | AVGREFNNLE |
| 3982-44/A1 | QGMVDGWYGY | HHSNEQGSGY | AADKESTQKA | IDGVTNKVNS | IIDKMNTQFE | AVGREFNNLE |
| 1553-2/B1 | QGMVDGWYGY | HHSNEQGSGY | AADKESTQKA | IDGVTNKVNS | IIDKMNTQFE | AVGREFNNLE |
| 1553-6/B1 | QGMVDGWYGY | HHSNXQGSGY | AADRESTQKA | IDGVTNKVNS | IIDKMNTQFE | AVGREFNNLE |
| 1553-13/B2 | QGMVDGWYGY | HHSNEQGSGY | AADKESTQKA | IDGVTNKVNS | IIDKMNTQFE | AVGREFNNLE |
| 1553-26/B2 | QGMVDGWYGY | HHSNEQGSGY | AADKESTQKA | IDGVTNKVNS | IIDKMNTQFE | AVGREFNNLE |
| 1553-28/B1 | QGMVDGWYGY | HHSNEQGTGY | AADKESTQKA | IDGVTNKVNS | IIDKMNTQFE | AVGREFNNLE |
| 2095-39/B2 | QGMVDGWYGY | HHSNEQGSGY | AADKESTQKA | IDGVTNKVNS | IIDKMNTQFE | AVGREFNNLE |
| 2095-46/B1 | QGMVDGWYGY | HHSNEQGTGY | AADKESTQKA | IDGVTNKVNS | IIDKMNTQFE | AVGREFNNLE |
| 2095-49/B1 | QGMVDGWYGY | HHSNEQGTGY | AADKESTQKA | IDGVTNKVNS | IIDKMNTQFE | AVGREFNNLE |
| 2095-65/B1 | QGMVDGWYGY | HHSNEQGTGY | AADKESTQKA | IDGVTNKVNS | IIDKMNTQFE | AVGREFNNLE |
| 2095-68/B2 | QGMVDGWYGY | HHSNEQGSGY | AADKESTQKA | IDGVTNKVNS | IIDKMNTQFE | AVGREFNNLE |
| 2095-70/B2 | QGMVDGWYGY | HHSNEQGSGY | AADKESTQKA | IDGVTNKVNS | IIDKMNTQFE | AVGREFNNLE |
| 2095-73/B2 | QGMVDGWYGY | HHSNEQGSGY | AADKESTQKA | IDGVTNKVNS | IIDKMNTQFE | AVGREFNNLE |
| 2095-75/B2 | QGMVDGWYGY | HHSNEQGSGY | AADKESTQKA | IDGVTNKVNS | IIDKMNTQFE | AVGREFNNLE |
| 3982-3/B1 | QGMVDGWYGY | HHSNEQGTGY | AADKESTQKA | IDGVTNKVNS | IIDKMNTQFE | AVGREFNNLE |
| 3982-4/B1 | QGMVDGWYGY | HHSNEQGTGY | AADKESTQKA | IDGVTNKVNS | IIDKMNTQFE | AVGREFNNLE |
| 3982-13/B1 | QGMVDGWYGY | HHSNEQGTGY | AADKESTQKA | IDGVTNKVNS | IIDKMNTQFE | AVGREFNNLE |
| 3982-14/B2 | QGMVDGWYGY | HHSNEQGSGY | AADKESTQKA | IDGVTNKVNS | IIDKMNTQFE | AVGREFNNLE |
| 3982-19/B3 | QGMVDGWYGY | HHSNEQGSGY | AADKESTQKA | IDGVTNKVNS | IIDKMNTQFE | AVGREFNNLE |
| 3982-21/B2 | QGMVDGWYGY | HHSNEQGSGY | AADKESTQKA | IDGVTNKVNS | IIDKMNTQFE | AVGREFNNLE |
| 3982-43/B1 | QGMVDGWYGY | HHSNEQGSGY | AADKESTQKA | INGVTNKVNS | IIDKMNTQFE | AVGREFNNLE |
| 3982-50/B1 | QGMVDGWYGY | HHSNEQGSGY | AADRESTQKA | IDGVTNKVNS | IIDKMNTQFE | AVGREFNNLE |
| 3982-52/B1 | QGMVDGWYGY | HHSNEQGSGY | AADKESTQKA | IDGVTNKVNS | IIDKMNTQFE | AVGREFNNLE |
| 3982-55/A1 | QGMVDGWYGY | HHSNEQGSGY | AADKESTQKA | IDGVTNKVNS | IIDKMNTQFE | AVGREFNNLE |
| 3982-56/A1 | QGMVDGWYGY | HHSNEQGSGY | AADKESTQKA | IDGVTNKVNS | IIDKMNTQFE | AVGREFNNLE |
| 3982-78/B2 | QGMVDGWYGY | HHSNEQGSGY | AADKESTQKA | IDGVTNKVNS | IIDKMNTQFE | AVGREFNNLE |
| 4794-17/B | QGMVDGWYGY | HHSNEQGSGY | AADKESTQKA | IDGVTNKVNS | IIDKMNTQFE | AVGREFNNLE |
| 4794-18/B | QGMVDGWYGY | HHSNEQGSGY | AADKESTQKA | IDGVTNKVNS | IIDKMNTQFE | AVGREFNNLE |
| | ....\|....\| | ....\|....\| | ....\|....\| | ....\|....\| | ....\|....\| | ....\|....\| |
| | 425 | 435 | 445 | 455 | 465 | 475 |
| 1553-1/A1 | RRIENLNKKM | EDGFLDVWTY | NAELLVLMEN | ERTLDFHDSN | VKNLYDKVRL | QLRDNAKELG |
| 1553-15/A1 | RRIENLNKKM | EDGFLDVWTY | NAELLVLMEN | ERTLDFHDSN | VKNLYDRVRL | QLRDNAKELG |
| 2095-50/A1 | RRIENLNKKM | EDGFLDVWTY | NAELLVLMEN | ERTLDFHDSN | VKNLYDKVRL | QLRDNAKELG |
| 3982-2/A1 | RRIENLNKKM | EDGFLDVWTY | NAELLVLMEN | ERTLDFHDSN | VKNLYDKVRL | QLRDNAKELG |
| 3982-5/A1 | RRIENLNKKM | EDGFLDVWTY | NAELLVLMEN | ERTLDFHDSN | VKNLYDKVRL | QLRDNAKELG |
| 3982-7/A1 | RRIENLNKKM | EDGFLDVWTY | NAELLVLMEN | ERTLDFHDSN | VKNLYDKVRL | QLRDNAKELG |
| 3982-8/A1 | RRIENLNKKM | EDGFLDVWTY | NAELLVLMEN | ERTLDFHDSN | VKNLYDKVRL | QLRDNAKELG |
| 3982-9/A1 | RRIENLNKKM | EDGFLDVWTY | NAELLVLMEN | ERTLDFHDSN | VKNLYDKVRL | QLRDNAKELG |
| 3982-12/A1 | RRIENLNKKM | EDGFLDVWTY | NAELLVLMEN | ERTLDFHDSN | VKNLYDKVRL | QLRDNAKELG |
| 3982-20/A1 | RRIENLNKKM | EDGFLDVWTY | NAELLVLMEN | ERTLDFHDSN | VKNLYDKVRL | QLRDNAKELG |
| 3982-44/A1 | RRIENLNKKM | EDGFLDVWTY | NAELLVLMEN | ERTLDFHDSN | VKNLYDKVRL | QLRDNAKELG |
| 1553-2/B1 | KRIENLNKKM | EDGFLDVWTY | NAELLVLMEN | ERTLDFHDSN | VRNLYDKVRL | QLRDNAKELG |
| 1553-6/B1 | KRIENLNKKM | EDGFLDVWTY | NAELLVLMEN | ERTLDFHDSN | VKNLYDKVRL | QLRDNAKELG |
| 1553-13/B2 | KRIENLNKKM | EDGFLDVWTY | NAELLVLMEN | ERTLDFHDSN | VKNLYDKVRL | QLRDNAKELG |
| 1553-26/B2 | KRIENLNKKM | EDGFLDVWTY | NAELLVLMEN | ERTLDFHDSN | VKNLYDKVRL | QLRDNAKELG |
| 1553-28/B1 | KRIENLNKKM | EDGFLDVWTY | NAELLVLMEN | ERTLDFHDSN | VRNLYDKVRL | QLRDNAKELG |
| 2095-39/B2 | KRIENLNKKM | EDGFLDVWTY | NAELLVLMEN | ERTLDFHDSN | VKNLYDKVRL | QLRDNAKELG |
| 2095-46/B1 | KRIENLNKKM | EDGFLDVWTY | NAELLVLMEN | ERTLDFHDSN | VRNLYDKVRL | QLRDNAKELG |
| 2095-49/B1 | KRIENLNKKM | EDGFLDVWTY | NAELLVLMEN | ERTLDFHDSN | VRNLYDKVRL | QLRDNAKELG |
| 2095-65/B1 | KRIENLNKKM | EDGFLDVWTY | NAELLVLMEN | ERTLDFHDSN | VRNLYDKVRL | QLRDNAKELG |
| 2095-68/B2 | KRIENLNKKM | EDGFLDVWTY | NAELLVLMEN | ERTLDFHDSN | VKNLYDKVRL | QLRDNAKELG |
| 2095-70/B2 | KRIENLNKKM | EDGFLDVWTY | NAELLVLMEN | ERTLDFHDSN | VKNLYDKVRL | QLRDNAKELG |
| 2095-73/B2 | KRIENLNKKM | EDGFLDVWTY | NAELLVLMEN | ERTLDFHDSN | VKNLYDKVRL | QLRDNAKELG |
| 2095-75/B2 | KRIENLNKKM | EDGFLDVWTY | NAELLVLMEN | ERTLDFHDSN | VKNLYDKVRL | QLRDNAKELG |
| 3982-3/B1 | KRIENLNKKM | EDGFLDVWTY | NAELLVLMEN | ERTLDFHDSN | VRNLYDKVRL | QLRDNAKELG |
| 3982-4/B1 | KRIENLNKKM | EDGFLDVWTY | NAELLVLMEN | ERTLDFHDSN | VRNLYDKVRL | QLRDNAKELG |
| 3982-13/B1 | KRIENLNKKM | EDGFLDVWTY | NAELLVLMEN | ERTLDFHDSN | VRNLYDKVRL | QLRDNAKELG |
| 3982-14/B2 | KRIENLNKKM | EDGFLDVWTY | NAELLVLMEN | ERTLDFHDSN | VKNLYDKVRL | QLRDNAKELG |
| 3982-19/B3 | RRIENLNKKM | EDGFLDVWTY | NAELLVLMEN | ERTLDFHDSN | VKNLYDKVRL | QLRDNAKELG |
| 3982-21/B2 | KRIENLNKKM | EDGFLDVWTY | NAELLVLMEN | ERTLDFHDSN | VKNLYDKVRL | QLRDNAKELG |
| 3982-43/B1 | KRIENLNKKM | EDGFLDVWTY | NAELLVLMEN | ERTLDFHDSN | VRNLYDKVRL | QLRDNAKELG |
| 3982-50/B1 | KRIENLNKKM | EDGFLDVWTY | NAELLVLMEN | ERTLDFHDSN | VKNLYDKVRL | QLRDNAKELG |
| 3982-52/B1 | KRIENLNKKM | EDGFLDVWTY | NAELLVLMEN | ERTLDFHDSN | VRNLYDKVRL | QLRDNAKELG |
| 3982-55/A1 | RRIENLNKKM | EDGFLDVWTY | NAELLVLMEN | ERTLDFHDSN | VKNLYDKVRL | QLRDNAKELG |
| 3982-56/A1 | RRIENLNKKM | EDGFLDVWTY | NAELLVLMEN | ERTLDFHDSN | VKNLYDKVRL | QLRDNAKELG |
| 3982-78/B2 | KRIENLNKKM | EDGFLDVWTY | NAELLVLMEN | ERTLDFHDSN | VKNLYDKVRL | QLRDNAKELG |
| 4794-17/B | KRIENLNKKM | EDGFLDVWTY | NAELLVLMEN | ERTLDFHDSN | VKNLYDKVRL | QLRDNAKELG |
| 4794-18/B | KRIENLNKKM | EDGFLDVWTY | NAELLVLMEN | ERTLDFHDSN | VKNLYDKVRL | QLRDNAKELG |
| | ....\|....\| | ....\|....\| | ....\|....\| | ....\|....\| | ....\|....\| | ....\|....\| |
| | 485 | 495 | 505 | 515 | 525 | 535 |
| 1553-1/A1 | NGCFEFYHRC | DNECMESVRN | GTYDYPQYSE | EARLKREEIS | GVKLESIGTY | QILSIYSTVA |
| 1553-15/A1 | NGCFEFYHRC | DNECMESVRN | GTYDYPQYSE | EARLKREEIS | GVKLESIGTY | QILSIYSTVA |
| 2095-50/A1 | NGCFEFYHRC | DNECMESVRN | GTYDYPQYSE | EARLKREEIS | GVKLESIGTY | QILSIYSTVA |
| 3982-2/A1 | NGCFEFYHRC | DNECMESVRN | GTYDYPQYSE | EARLKREEIS | GVKLESIGTY | QILSIYSTVA |
| 3982-5/A1 | NGCFEFYHRC | DNECMESVRN | GTYDYPQYSE | EARLKREEIS | GVKLESIGTY | QILSIYSTVA |
| 3982-7/A1 | NGCFEFYHRC | DNECMESVRN | GTYDYPQYSE | EARLKREEIS | GVKLESIGTY | QILSIYSTVA |
| 3982-8/A1 | NGCFEFYHRC | DNECMESVRN | GTYDYPQYSE | EARLKREEIS | GVKLESIGTY | QILSIYSTVA |
| 3982-9/A1 | NGCFEFYHRC | DNECMESVRN | GTYDYPQYSE | EARLKREEIS | GVKLESIGTY | QIL------- |
| 3982-12/A1 | NGCFEFYHRC | DNECMESVRN | GTYDYPQYSE | EARLKREEIS | GVKLESIGTY | QILSIYSTVA |
| 3982-20/A1 | NGCFEFYHRC | DNECMESVRN | GTYDYPQYSE | EARLKREEIS | GVKLESIGTY | QILSIYX--- |
| 3982-44/A1 | NGCFEFYHRC | DNECMESVRN | GTYDYPQYSE | EARLKREEIS | GVKLESIGTY | QILSIYSTVA |
| 1553-2/B1 | NGCFEFYHRC | DNECMESVRN | GTYDYPQYSE | EARLKREEIS | GVKLESIGTY | QILSIYSTVA |
| 1553-6/B1 | NGCFEFYHRC | DNECIESVRN | GTYDYPQYSE | EARLKREEIS | GVKLESIGTY | QILSIYSTVA |
| 1553-13/B2 | NGCFEFYHRC | DNECMESVRN | GTYDYPQYSE | EARLKREEIS | GVKLESIGTY | QILSIYSTVA |
| 1553-26/B2 | NGCFEFYHRC | DNECMESVRN | GTYDYPQYSE | EARLKREEIS | GVKLESIGTY | QILSIYSTVA |
| 1553-28/B1 | NGCFEFYHKC | DNECMESVRN | GTYDYPQYSK | EARLKREEIS | GVKLESIGTY | QILSIYSTVT |
| 2095-39/B2 | NGCFEFYHRC | DNECMESVRN | GTYDYPQYSE | EARLKREEIS | GVKLESIGTY | QILSIYSTVA |
| 2095-46/B1 | NGCFEFYHKC | DNECMESVRN | GTYDYPQYSK | EARLKREEIS | GVKLESIGTY | QILSIYSTVA |
| 2095-49/B1 | NGCFEFYHKC | DNECMESVRN | GTYDYPQYSK | EARLKREEIS | GVKLESIGTY | QILSIYSTVA |
| 2095-65/B1 | NGCFEFYHKC | DNECMESVRN | GTYDYPQYSK | EARLKREEIS | GVKLESIGTY | QILSIYSTVA |
| 2095-68/B2 | NGCFEFYHRC | DNECMESVRN | GTYDYPQYSE | EARLNREEIS | GVKLESIGTY | QILSIYSTVA |
| 2095-70/B2 | NGCFEFYHRC | DNECMESVRN | GTYDYPQYSE | EARLKREEIS | GVKLESIGTY | QILSIYSTVA |
| 2095-73/B2 | NGCFEFYHRC | DNECMESVRN | GTYDYPQYSE | EARLKREEIS | GVKLESIGTY | QILSIYSTVA |
| 2095-75/B2 | NGCFEFYHRC | DNECMESVRN | GTYDYPQYSE | EARLKREEIS | GVKLESIGTY | QILSIYSTVA |
| 3982-3/B1 | NGCFEFYHKC | DNECMESVRN | GTYDYPQYSK | EARLKREEIS | GVKLESIGTY | QILSIYSTVA |
| 3982-4/B1 | NGCFEFYHKC | DNECMESVRN | GTYDYPQYSK | EARLKREEIS | GVKLESIGTY | QILSIYSTVA |
| 3982-13/B1 | NGCFEFYHKC | DNECMESVRN | GTYDYPQYSK | EARLKREEIS | GVKLESIGTY | QILSIYSTVA |
| 3982-14/B2 | NGCFEFYHRC | DNECMESVRN | GTYDYPQYSE | EARLKREEIS | GVKLESIGTY | QILSIYSTVA |
| 3982-19/B3 | NGCFEFYHRC | DNECMESVRN | GTYDYPQYSE | EARLKREEIS | GAKLESIGTY | QIX------- |
| 3982-21/B2 | NGCFEFYHRC | DNECMESVKN | GTYDYPQYSE | EARLKREEIS | GVKLESIGTY | QILSIYSTVA |
| 3982-43/B1 | NGCFEFYHRC | DNECMESVRN | GTYDYPQYSK | EARLKREEIS | GVKLESIGTY | QILSIYSTVA |
| 3982-50/B1 | NGCFEFYHRC | DNECIESVRN | GTYDYPQYSE | EARLKREEIS | GVKLESIGTY | QILSIYSTVA |
| 3982-52/B1 | NGCFEFYHRC | DNECMESVRN | GTYDYPQYSE | EARLKREEIS | GVKLEX---- | ---------- |
| 3982-55/A1 | NGCFEFYHRC | DNECMESVRN | GTYDYPQYSE | EARLKREEIS | GVKLESIGTX | QI-------- |
| 3982-56/A1 | NGCFEFYHRC | DNECMESVRN | GTYDYPQYSE | EARLKREEIS | GVKLESIGTX | QIX------- |
| 3982-78/B2 | NGCFEFYHRC | DNECMESVRN | GTYDYPQYSE | EARLKREEIS | GVKLESIGTY | QIL*IYSTVA |
| 4794-17/B | NGCFEFYHRC | DNECMESVRN | GTYDYPQYSE | EARLKREEIS | GVKLESIGTY | QILSIYSTVA |
| 4794-18/B | NGCFEFYHRC | DNECMESVRN | GTYDYPQYSE | EARLKREEIS | GVKLESIGTY | QILSIYSTVA |
| | ....\|....\| | ....\|....\| | ....\|.... | | | |
| | 545 | 555 | 565 | | | |
| 1553-1/A1 | SSLALAIMVA | GLFLWMCSNG | SLQCRICI* | | | |
| 1553-15/A1 | SSLALAIMVA | GLFLWMCSNG | SLQCRICI* | | | |
| 2095-50/A1 | SSLALAIMVA | GLFLWMCSNG | X-------- | | | |
| 3982-2/A1 | SSLALAIMVX | ---------- | --------- | | | |
| 3982-5/A1 | SSLALAIM-- | ---------- | --------- | | | |
| 3982-7/A1 | SSLALAIMVA | GLFLWMCSNG | X-------- | | | |
| 3982-8/A1 | SSLALAIMVX | ---------- | --------- | | | |
| 3982-9/A1 | ---------- | ---------- | --------- | | | |
| 3982-12/A1 | SSLALAIX-- | ---------- | --------- | | | |
| 3982-20/A1 | ---------- | ---------- | --------- | | | |
| 3982-44/A1 | SSLALAIX-- | ---------- | --------- | | | |
| 1553-2/B1 | SSLALAIMVA | GLFLWMCSNG | SLQCRICI* | | | |
| 1553-6/B1 | SSLALAIIVA | GLFLWMCSNG | SLQCRICI* | | | |
| 1553-13/B2 | SSLALAIMMA | GLFLWMCSNG | SLQCRICI* | | | |
| 1553-26/B2 | SSLALAIMMA | GLFLWMCSNG | SLQCRICI* | | | |
| 1553-28/B1 | SSLALAIMVA | GLSLWMCSNG | SLQCRICI* | | | |
| 2095-39/B2 | SSLALAIMMA | GLFLWX---- | --------- | | | |
| 2095-46/B1 | SSLALAIMVA | GLSLWMCSX- | --------- | | | |
| 2095-49/B1 | SSLALAIMVA | GLSLWMCSNG | SLQCRICI- | | | |
| 2095-65/B1 | SSLALAIMVA | GLSLWM---- | --------- | | | |
| 2095-68/B2 | SSLALAIMMA | GLFLWMCSNG | X-------- | | | |
| 2095-70/B2 | GSLALAIMMA | GLFLWMCSNG | SLQ------ | | | |
| 2095-73/B2 | SSLALAIMVX | ---------- | --------- | | | |
| 2095-75/B2 | GSLALAIMVX | ---------- | --------- | | | |
| 3982-3/B1 | SSLALAIMVA | GLSLWMCSNG | SLQ------ | | | |
| 3982-4/B1 | SSLALAIMVA | GLSLWMCSNG | SLQCRX--- | | | |
| 3982-13/B1 | SSLALAIMVA | X--------- | --------- | | | |
| 3982-14/B2 | SSLALAIMVA | GLFLWMCSNG | SLQCRICI* | | | |
| 3982-19/B3 | ---------- | ---------- | --------- | | | |
| 3982-21/B2 | SSLALAIM-- | ---------- | --------- | | | |
| 3982-43/B1 | SSLALAIM-- | ---------- | --------- | | | |
| 3982-50/B1 | SSLALAIM-- | ---------- | --------- | | | |
| 3982-52/B1 | ---------- | ---------- | --------- | | | |
| 3982-55/A1 | ---------- | ---------- | --------- | | | |
| 3982-56/A1 | ---------- | ---------- | --------- | | | |
| 3982-78/B2 | SSLALAX--- | ---------- | --------- | | | |
| 4794-17/B | SSLALAI--- | ---------- | --------- | | | |
| 4794-18/B | SSLALAIMMA | GLFLWMCSNG | X-------- | | | |

TABLE C
The following shows SEQ ID NOs: 100 to 137, which correspond to
the variant sequences as set forth in SEQ ID NOs: 9 to 46 starting with
the N-terminal amino acid D17 and having a full length C-terminal sequence,
wherein the numbering of said first N-terminal amino acid position (D17)
refers to the amino acid position 17 as exemplarily given in SEQ ID NO 8,
and wherein the full length C-terminal sequence corresponds to amino
acids 527 to 568, wherein the numbering of said amino acid positions
refers to the amino acid positions 527 to 568 as exemplarily given
in SEQ ID NO: 47:

| | ....\|....\| | ....\|....\| | ....\|....\| | ....\|....\| | ....\|....\| | ....\|....\| |
|---|---|---|---|---|---|---|
| | 5 | 15 | 25 | 35 | 45 | 55 |
| 1553-1/A1 | (SEQ ID NO: 100) | DQIC | IGYHANNSTE | QVDTIMEKNV | TVTHAQDILE | KTHNGKLCNL |
| 1553-15/A1 | (SEQ ID NO: 101) | DQIC | IGYHANNSTE | QVDTIMEKNV | TVTHAQDILE | KTHNGKLCNL |
| 2095-50/A1 | (SEQ ID NO: 102) | DQIC | IGYHANNSTE | QVDTIMEKNV | TVTHAQDILE | KTHNGKLCNL |
| 3982-2/A1 | (SEQ ID NO: 103) | DQIC | IGYHANNSTE | QVDTIMEKNV | TVTHAQDILE | KTHNGKLCNL |
| 3982-5/A1 | (SEQ ID NO: 104) | DQIC | IGYHANNSTE | QVDTIMEKNV | TVTHAQDILE | KTHNGKLCNL |
| 3982-7/A1 | (SEQ ID NO: 105) | DQIC | IGYHANNSTE | QVDTIMEKNV | TVTHAQDILE | KTHNGKLCNL |
| 3982-8/A1 | (SEQ ID NO: 106) | DQIC | IGYHANNSTE | QVDTIMEKNV | TVTHAQDILE | KTHNGKLCNL |
| 3982-9/A1 | (SEQ ID NO: 107) | DQIC | IGYHANNSTE | QVDTIMEKNV | TVTHAQDILE | KTHNGKLCNL |
| 3982-12/A1 | (SEQ ID NO: 108) | DQIC | IGYHANNSTE | QVDTIMEKNV | TVTHAQDILE | KTHNGKLCNL |
| 3982-20/A1 | (SEQ ID NO: 109) | DQIC | IGYHANNSTE | QVDTIMEKNV | TVTHAQDILE | KTHNGKLCNL |
| 3982-44/A1 | (SEQ ID NO: 110) | DQIC | IGYHANNSTE | QVDTIMEKNV | TVTHAQDILE | KTHNGKLCNL |
| 1553-2/B1 | (SEQ ID NO: 111) | DQIC | IGYHANNSTE | QVDTIMEKNV | TVTHAQDILE | KTHNGKLCDL |
| 1553-6/B1 | (SEQ ID NO: 112) | DQIC | IGYHANNSTE | QVDTIMEKNV | TVTHAQDILE | KTHNGKLCDL |
| 1553-13/B2 | (SEQ ID NO: 113) | DQIC | IGYHANNSTE | QVDTIMEKNV | TVTHAQDILE | KTHNGKLCDL |
| 1553-26/B2 | (SEQ ID NO: 114) | DQIC | IGYHANNSTE | QVDTIMEKNV | TVTHAQDILE | KTHNGKLCDL |
| 1553-28/B1 | (SEQ ID NO: 115) | DQIC | IGYHANNSTE | QVDTIMEKNV | TVTHAQDILE | KTHNGKLCDL |
| 2095-39/B2 | (SEQ ID NO: 116) | DQIC | IGYHANNSTE | QVDTIMEKNV | TVTHAQDILE | KTHNGKLCDL |
| 2095-46/B1 | (SEQ ID NO: 117) | DQIC | IGYHANNSTE | QVDTIMEKNV | TVTHAQDILE | KTHNGKLCDL |
| 2095-49/B1 | (SEQ ID NO: 118) | DQIC | IGYHANNSTE | QVDTIMEKNV | TVTHAQDILE | KTHNGKLCDL |
| 2095-65/B1 | (SEQ ID NO: 119) | DQIC | IGYHANNSTE | QVDTIMEKNV | TVTHAQDILE | KTHNGKLCDL |
| 2095-68/B2 | (SEQ ID NO: 120) | DQIC | IGYHANNSTE | QVDTIMEKNV | TVTHAQDILE | KTHNGKLCDL |
| 2095-70/B2 | (SEQ ID NO: 121) | DQIC | IGYHANNSTE | QVDTIMEKNV | TVTHAQDILE | KTHNGKLCDL |
| 2095-73/B2 | (SEQ ID NO: 122) | DQIC | IGYHANNSTE | QVDTIMEKNV | TVTHAQDILE | KTHNGKLCDL |
| 2095-75/B2 | (SEQ ID NO: 123) | DQIC | IGYHANNSTE | QVDTIMEKNV | TVTHAQDILE | KTHNGKLCDL |
| 3982-3/B1 | (SEQ ID NO: 124) | DQIC | IGYHANNSTE | QVDTIMEKNV | TVTHAQDILE | KTHNGKLCDL |
| 3982-4/B1 | (SEQ ID NO: 125) | DQIC | IGYHANNSTE | QVDTIMEKNV | TVTHAQDILE | KTHNGKLCDL |
| 3982-13/B1 | (SEQ ID NO: 126) | DQIC | IGYHANNSTE | QVDTIMEKNV | TVTHAQDILE | KTHNGKLCDL |
| 3982-14/B2 | (SEQ ID NO: 127) | DQIC | IGYHANNSTE | QVDTIMEKNV | TVTHAQDILE | KTHNGKLCDL |
| 3982-19/B3 | (SEQ ID NO: 128) | DQIC | IGXHANNSTE | QVDTIMEKNI | TVTHAQDILE | KTHNGKLCDL |
| 3982-21/B2 | (SEQ ID NO: 129) | DQIC | IGYHANNSTE | QVDTIMEKNV | TVTHAQDILE | KTHNGKLCDL |
| 3982-43/B1 | (SEQ ID NO: 130) | DQIC | IGYHANNSTE | QVDTIMEKNV | TVTHAQDILE | KTHNGKLCDL |
| 3982-50/B1 | (SEQ ID NO: 131) | DQIC | IGYHANNSTE | QVDTIMEKNV | TVTHAQDILE | KTHNGKLCDL |
| 3982-52/B1 | (SEQ ID NO: 132) | DQIC | IGYHANNSTE | QVDTIMEKNV | TVTHAQDILE | KTHNGKLCDL |
| 3982-55/A1 | (SEQ ID NO: 133) | DQIC | IGYHANNSTE | QVDTIMEKNV | TVTHAQDILE | KTHNGKLCNL |
| 3982-56/A1 | (SEQ ID NO: 134) | DQIC | IGXHANNSTE | QVDTIMEKNV | TVTHAQDILE | KTHNGKLCNL |
| 3982-78/B2 | (SEQ ID NO: 135) | DQIC | IGYHANNSTE | QVDTIMEKNV | TVTHAQDILE | KTHNGKLCDL |
| 4794-17/B | (SEQ ID NO: 136) | DQIC | IGYHANNSTE | QVDTIMEKNV | TVTHAQDILE | KTHNGKLCDL |
| 4794-18/B | (SEQ ID NO: 137) | DQIC | IGYHANNSTE | QVDTIMEKNV | TVTHAQDILE | KTHNGKLCDL |
| | ....\|....\| | ....\|....\| | ....\|....\| | ....\|....\| | ....\|....\| | ....\|....\| |
| | 65 | 75 | 85 | 95 | 105 | 115 |
| 1553-1/A1 | DGVKPLILRD | CSVAGWLLGN | PMCDEFLNVP | EWSYIVEKIN | PANDLCYPGK | FNDYEELKHL |
| 1553-15/A1 | DGVKPLILRD | CSVAGWLLGN | PMCDEFLNVP | EWSYIVEKIN | PANDLCYPGN | FNDYEELKHL |
| 2095-50/A1 | DGVKPLILRD | CSVAGWLLGN | PMCDEFLNVP | EWSYIVEKIY | PANDLCYPGN | FNDYEELKHL |
| 3982-2/A1 | DGVKPLILRD | CSVAGWLLGN | PMCDEFLDVP | EWSYIVEKIN | PANDLCYPGN | FNDYEELKHL |
| 3982-5/A1 | DGVKPLILRD | CSVAGWLLGN | PMCDEFLNV | EWSYIVEKIN | PTNDLCYPGN | FNDYEELKHL |
| 3982-7/A1 | DGVKPLILRD | CSVAGWLLGN | PMCDKFLNVP | EWSYIVEKIN | PTNDLCYPGN | FNDYEELKHL |
| 3982-8/A1 | DGVKPLILRD | CSVAGWLLGN | PMCDEFLNVP | EWSYIVEKIN | PTNDLCYPGN | FNDYEELKHL |
| 3982-9/A1 | DGVKPLILRD | CSVAGWLLGN | PMCDEFLNVP | EWSYIVEKIN | PANDLCYPGN | FNDYEELKHL |
| 3982-12/A1 | DGVKPLILRD | CSVAGWLLGN | PMCDEFLNVP | EWSYIVEKIN | PANDLCYPGN | FNDYEELKHL |
| 3982-20/A1 | DGVKPLILRD | CSVAGWLLGN | PMCDEFLNVP | EWSYIVEKIN | PANDLCYPGN | FNDYEELKHL |
| 3982-44/A1 | DGVKPLILRD | CSVAGWLLGN | PMCDEFLNVP | EWSYIVEKIN | PANDLCYPGN | FNDYEELKHL |
| 1553-2/B1 | GGVKPLILRD | CSVAGWLLGN | PMCDEFPNVS | EWSYIVEKIN | PANDLCYPGN | FNNYEELKHL |
| 1553-6/B1 | DGVKPLILRD | CSVAGWLLGN | PMCDEFPNVS | EWSYIVEKTN | PANDLCYPGN | FNNYEELKHL |
| 1553-13/B2 | DGVKPLILRD | CSVAGWLLGN | PMCDEFPNVS | EWSYIVEKIN | PANDLCYPGN | FNNYEELKHL |
| 1553-26/B2 | DGVKPLILRD | CSVAGWLLGN | PMCDEFPNVS | EWSYIVEKIN | PANDLCYPGN | FNNYEELKHL |
| 1553-28/B1 | DGVKPLILRD | CSVAGWLLGN | PMCDEFPNVS | EWSYIVEKIN | PANDLCYPGN | FNNYEELKHL |
| 2095-39/B2 | DGVKPLILRD | CSVAGWLLGN | PMCDEFPNVS | EWSYIVEKTN | PANDLCYPGN | FNNYEELKHL |
| 2095-46/B1 | DGVKPLILRD | CSVAGWLLGN | PMCDEFPNVS | EWSYIVEKIN | PANDLCYPGN | FNNYEELKHL |
| 2095-49/B1 | DGVKPLILRD | CSVAGWLLGN | PMCDEFPNVS | EWSYIVEKIN | PANDLCYPGN | FNNYEELKHL |
| 2095-65/B1 | DGVKPLILRG | CSVAGWLLGN | PMCDEFPNVS | EWSYIVEKIN | PANDLCYPGN | FNNYEELKHL |
| 2095-68/B2 | DGVKPLILRD | CSVAGWLLGN | PMCDEFPNVS | EWSYIVEKIN | PANDLCYPGN | FNNYEELKHL |
| 2095-70/B2 | DGVKPLILRD | CSVAGWLLGN | PMCDEFPNVS | EWSYIVEKIN | PANDLCYPGN | FNNYEELKHL |
| 2095-73/B2 | DGVKPLILRD | CSVAGWLLGN | PMCDEFPNVS | EWSYIVEKIN | PANDLCYPGN | FNNYEELKHL |
| 2095-75/B2 | DGVKPLILRD | CSVAGWLLGN | PMCDEFPNVS | EWSYIVEKIN | PANDLCYPGN | FNNYEELKHL |
| 3982-3/B1 | DGVKPLILRD | CSVAGWLLGN | PMCDEFPNVS | EWSYIVEKIN | PANDLCYPGN | FNNYEELKHL |
| 3982-4/B1 | DGVKPLILRD | CSVAGWLLGN | PMCDEFPNVS | EWSYIVEKIN | PANDLCYPGN | FNNYEELKHL |
| 3982-13/B1 | DGVKPLILRD | CSVAGWLLGN | PMCDEFPNVS | EWSYIVEKIN | PANDLCYPGN | FNNYEELKHL |
| 3982-14/B2 | DGVKPLILRD | CSVAGWLLGN | PMCDEFPNVS | EWSYIVEKIN | PANDLCYPGN | FNNYEELKHL |
| 3982-19/B3 | DGVKPLILRD | CSVAGWLLGN | PMCDEFLNVS | EWSYIVEKIN | PANDLCYPGN | FNNYEELKHL |
| 3982-21/B2 | DGVKPLILRD | CSVAGWLLGN | PMCDEFPNVS | EWSYIVEKIN | PANDLCYPGN | FNNYEELKHL |
| 3982-43/B1 | DGVKPLILRD | CSVAGWLLGN | PMCDEFPNVS | EWSYIVEKIN | PANDLCYPGN | FNNYEELKHL |
| 3982-50/B1 | DGVKPLILRD | CSVAGWLLGN | PMCDEFPNVS | EWSYIVEKTN | PANDLCYPGN | FNNYEELKHL |
| 3982-52/B1 | DGVKPLILRD | CSVAGWLLGN | PMCDEFPNVS | EWSYIVEKIN | PANDLCYPGN | FNNYEELKHL |
| 3982-55/A1 | DGVKPLILRD | CSVAGWLLGN | PMCDKFLNVP | EWSYIVEKIN | PANDLCYPGD | FNDYEELKHL |
| 3982-56/A1 | DGVKPLILRD | CSVAGWLLGN | PMCDEFPNVL | EWSYIVEKIN | PANDLCYPGN | FNDYEELKHL |
| 3982-78/B2 | DGVKPLILRD | CSVAGWLLGN | PMCDEFPNVS | EWSYIVEKIN | PANDLCYPGN | FNNYEELKHL |
| 4794-17/B | DGVKPLILRD | CSVAGWLLGN | PMCDEFPNVS | EWSYIVEKIN | PANDLCYPGN | FNNYEELKHL |
| 4794-18/B | DGVKPLILRD | CSVAGWLLGN | PMCDEFPNVS | EWSYIVEKIN | PANDLCYPGN | FNNYEELKHL |
| | ....\|....\| | ....\|....\| | ....\|....\| | ....\|....\| | ....\|....\| | ....\|....\| |
| | 125 | 135 | 145 | 155 | 165 | 175 |
| 1553-1/A1 | LSRINHFEKI | QIIPRNSWSD | HETS-GVSSA | CQYQGRSSFF | RNVVWLTKKD | NAYSTIKRSY |
| 1553-15/A1 | LSRINHFEKI | QIIPKGSWSD | HEAS-GVSSA | CPYQGRSSFF | RNVVWLTKKN | NAYPTIKKSY |
| 2095-50/A1 | LSRINHFEKI | QIIPKSSWSD | HEAS-GVSSA | CPYQGRSSFF | RNVVWLTKKD | NAYPTIKKSY |
| 3982-2/A1 | LSRINHFEKI | QIIPKNSWSD | HETS-GVSSA | CPYQGRSSFF | RNVVWLTKKN | TAYPTIKKSY |
| 3982-5/A1 | LSRINHFEKI | QIIPKNYWSD | HETS-GVSSA | CPYQGRPSFF | RNVVWLTKKN | NAYPTIKKSY |
| 3982-7/A1 | LSRINHFEKI | QIIPKNSWSD | HEAS-GVSSA | CPYQGRSSFF | RNVVWLTKKN | NAYPTIKKSY |
| 3982-8/A1 | LSRINHFEKI | QIIPKNSWSD | HEAS-GVSSA | CPYQGRSSFF | RNVVWLTKKN | NAYPTIKKSY |
| 3982-9/A1 | LSRINHFEKI | QIIPKNSWSD | HEAS-GVSSA | CPYQGRSSFF | RNVVWLTKKN | NXYPTIKKSY |
| 3982-12/A1 | LSRINHFEKI | QIIPKNSWSD | HEAS-GVSSA | CPYQGRSSFF | RNVVWLTKKN | NAYPTIKKSY |
| 3982-20/A1 | LSRINHFEKI | QIIPKNSWSD | HEAS-GVSSA | CPYQGRSSFF | RNVVWLTKKN | NAYPTIKKSY |
| 3982-44/A1 | LSRINHFEKI | QIIPKNSWSD | HEAS-GVSSA | CPYQGRSSFF | RNVVWLTKKD | NAYPTIKRSY |
| 1553-2/B1 | LSRINRFEKI | QIIPKSSWPD | HEASLGVSSA | CPYQGGPSFY | RNVVWLIKKN | DTYPTIKESY |
| 1553-6/B1 | LSRINRFEKI | KIIPKSSWPD | HEASLGVSSA | CPYQGEPSFY | RNVVWLIKKN | NTYPTIKESY |
| 1553-13/B2 | LSRINRFEKI | KIIPKSSWPD | HEASLGVSSA | CPYQRGPSFY | RNVVWLIKKN | NTYPTIKKSY |
| 1553-26/B2 | LSRINRFEKI | KIIPKSSWPD | HEASLGVSSA | CPYQGGPSFY | RNVVWLIKKN | NTYPTIKESY |
| 1553-28/B1 | LSRINRFEKI | QIIPKSSWPD | HEASLGVSSA | CPYQGEPSFY | RNVVWLIKKN | NTYPTIKESY |
| 2095-39/B2 | LSRINRFEKI | KIIPKSSWPD | HEASLGVSSA | CPYQGRPSFY | RNVVWLIKKN | NTYPTIKESY |
| 2095-46/B1 | LSRINRFEKI | QIIPKSSWPD | HEASLGVSSA | CPYQGEPSFY | RNVVWLIKKN | NTYPTIKENY |
| 2095-49/B1 | LSRINRFEKI | QIIPKSSWPD | HEASLGVSSA | CPYQGEPSFY | RNVVWLIKKN | NTYPTIKENY |
| 2095-65/B1 | LSRINRFEKI | QIIPKSSWPD | HEASLGVSSA | CPYQGEPSFY | RNVVWLIKKN | NTYPTIKESY |
| 2095-68/B2 | LSRINRFEKI | KIIPKSSWPD | HEASLGVSSA | CPYQGGPSFY | RNVVWLIKKN | NTYPTIKKSY |
| 2095-70/B2 | LSRINRFEKI | KIIPKXSWPD | HEASLGVSSA | CPYQGGPSFY | RNVVWLIKKN | NTYPTIKESY |
| 2095-73/B2 | LSRINRFEKI | KIIPKSSWPD | HEASLGVSSA | CPYQEGPSFY | RNVVWLIKKN | NTYPTIKKSY |
| 2095-75/B2 | LSRINRFEKI | KIIPKSSWPD | HEASLGVSSA | CPYQGGXSFY | RNVVWLIKKN | NTYPTIKESY |
| 3982-3/B1 | LSRINRFEKI | QIIPKSSWPD | HEASLGVSSA | CPYQGEPSFY | RNVVWLIKKN | NTYPTIKENY |
| 3982-4/B1 | LSRINRFEKI | QIIPKSSWPD | HEASLGVSSA | CPYQGEPSFY | RNVVWLIKKN | NTYPTIKESY |
| 3982-13/B1 | LSRINRFEKI | QIIPKSSWPD | HEASLGVSSA | CPYQGEPSFY | RNVVWLIKKN | NTYPTIKENY |
| 3982-14/B2 | LSRINRFEKI | KIIPKSSWPD | HEASLGVSSA | CPYQGGPSFY | RNVVWLIKKN | NTYPTIKESY |
| 3982-19/B3 | LSRINRFEKI | QIISKNSWPD | HEASLGVSAA | CPYQGGLSFY | RNVVWLIEKN | NTYPLIKKNY |
| 3982-21/B2 | LSRINRFEKI | KIIPKSSWPD | HEASLGVSSA | CPYQRGPSFY | RNVVWLIKKN | NTYPTIKKSY |
| 3982-43/B1 | LSRINRFEKI | QIIPKSSWPD | HEASLGVSSA | CPYQGGPSFY | RNVVWLIKKN | DTYPTIKESY |
| 3982-50/B1 | LSRINRFEKI | KIIPKSSWPD | HEASLGVSSA | CPYQGEPSFY | RNVVWLIKKN | NTYPTIKESY |
| 3982-52/B1 | LSRINRFEKI | QIIPKSSWPD | HEASLGVSSA | CPYQGGPSFY | RNVVWLIKKN | DTYPTIKESY |
| 3982-55/A1 | LSRINHFEKI | QIIPKNSWSD | HEAS-GVSSA | CPYQGRSSFF | RNVVWLTKKN | NAYPTIKKSY |
| 3982-56/A1 | LSRINHFEKI | QIIPKNSWSD | HEAS-GVSSA | CPYQRRSSFF | RNVVWLTKKN | NAYPTIKKSY |
| 3982-78/B2 | LSRINRFEKI | KIIPKSSWPD | HEASLGVSSA | CPYQGGPSFY | RNVVWLIKKN | NTYPTIKKSY |
| 4794-17/B | LSRINRFEKI | KIIPKSSWPD | HEASLGVSSA | CPYQGGPSFY | RNVVWLIKKN | NTYPTIKESY |
| 4794-18/B | LSRINRFEKI | KIIPKSSWPD | HEASLGVSSA | CPYQGGPSFY | RNVVWLTKKN | NTYPTIKKSY |
| | ....\|....\| | ....\|....\| | ....\|....\| | ....\|....\| | ....\|....\| | ....\|....\| |
| | 185 | 195 | 205 | 215 | 225 | 235 |
| 1553-1/A1 | NNTNQEDLLV | LWGIHHPNDA | AEQTRLYQNP | TTYISVGTST | LNQRLVPKIA | TRSKVNGQSG |
| 1553-15/A1 | NNTNQEDLLV | LWGIHHPNDA | AEQTRLYQNP | TTYISVGTST | LNQRLVPKIA | TRSKVNGQSG |
| 2095-50/A1 | NNTNQEDLLI | LWGIHHPNDA | AEQTRLYQNP | TTYISVGTST | LNQRLVPKIA | TRSKVNGQSG |
| 3982-2/A1 | NNTNQEDLLV | LWGIHHPNDA | AEQTRLYQNP | TTYISVGTST | LNQRLVPKIA | TRSKVNGQSG |
| 3982-5/A1 | NNTNQEDLLV | LWGIHHPNDA | AEQTRLYQNP | TTYISVGTST | LNQRLVPKIA | TRSKVNGQSG |
| 3982-7/A1 | NNTNQEDLLV | LWGIHHPNDA | AEQTRLYQNP | TTYISVGTST | LNQRLVPKIA | TRSKVNGQSG |
| 3982-8/A1 | NNTNQEDLLV | LWGIHHPNDA | AEQTRLYQNP | TTYISVGTST | LNQRLVPKIA | TRSKVNGQSG |
| 3982-9/A1 | NNTNQEDLLV | LWGIHHPNDA | AEQTKLYQNP | TTYISVGTST | LNQRLVPKIA | TRSKVNGQSG |
| 3982-12/A1 | NNTNQEDLLV | LWGIHHPNDE | AEQTRLYQNP | TTYISVGTST | LNQRLVPKIA | TRSKVNGQSG |
| 3982-20/A1 | NNTNQEDLLV | LWGIHHPNDA | AEQTRLYQNP | TTYISVGTST | LNQRLVPKIA | NRSKVNGQSG |
| 3982-44/A1 | NNTNQEDLLV | LWGIHHPNDA | AEQTRLYQNP | TTYISVGTST | LNQRLVPKIA | TRSKVNGQSG |
| 1553-2/B1 | HNTNQEDLLV | LWGIHHPNNE | EEQKRIYKNP | TTYVSVGTST | LNQRLVPKIA | TRSKVNGQSG |
| 1553-6/B1 | HNTNQEDLLV | LWGIHHPNDE | EEQTRIYKNP | TTYISVGTST | LNQRLVPKIA | TRSKVNGQSG |
| 1553-13/B2 | HNTNQEDLLV | LWGIHHPNDE | EEQTRIYKNP | TTYISVGTST | LNQRLVPKIA | TRSKVNGQSG |
| 1553-26/B2 | HNTNQEDLLV | LWGIHHPNDE | EEQTRIYKNP | TTYISIGTST | LNQRLVPKIA | TRSKVNGQRG |
| 1553-28/B1 | HNTNQEDLLV | LWGIHHPNDE | EEQKRIYKNP | TTYISVGTST | LNQRLVPKIA | TRPKVNGQSG |
| 2095-39/B2 | HNTNQEDLLV | LWGIHHPNDE | EEQTRIYKNP | TTYISIGTST | LNQRLIPKIA | TRSKVNGQSG |
| 2095-46/B1 | HNTNQEDLLV | LWGIHHPNDE | EEQKRIYKNP | TTYISVGTST | LNQRLVPKIA | TRPKVNGQSG |
| 2095-49/B1 | HNTNQEDLLV | LWGIHHPNDE | EEQKRIYKNP | TTYISVGTST | LNQRLVPKIA | TRPKVNGQSG |
| 2095-65/B1 | HNTNQEDLLV | LWGIHHPNDE | EEQKRIYKNP | TTYISVGTST | LNQRLVPKIA | TRPKVNGQSG |
| 2095-68/B2 | HNTNQEDLLV | LWGIHHPNDE | EEQTRIYKNP | TTYISVGTST | LNQRLVPKIA | TRSKVNGQSG |
| 2095-70/B2 | HNTNQEDLLV | LWGIHHPNDE | EEQTRIYKNP | TTYISIGTST | LNQRLVPKIA | TRSKVNGQSG |
| 2095-73/B2 | HNTNQEDLLV | LWGIHHPNDE | EEQTRIYKNP | TTYISVGTST | LNQRLVPKIA | TRSKVNGQSG |
| 2095-75/B2 | HNTNQEDLLV | LWGIHHPNDE | EEQTRIYKNP | TTYISIGTST | LNQRLVPKIA | TRSKVNGQSG |
| 3982-3/B1 | HNTNQEDLLV | LWGIHHPNDE | EEQKRIYKNP | TTYISVGTST | LTQRLVPKIA | TRPKVNGQSG |
| 3982-4/B1 | HNTNQEDLLV | LWGIHHPNDE | EEQKRIYKNP | TTYISVGTST | LNQRLVPKIA | TRPKVNGQSG |
| 3982-13/B1 | HNTNQEDLLV | LWGIHHPNDE | EEQKRIYKNP | TTYISVGTST | LNQRLVPKIA | TRPKVNGQSG |
| 3982-14/B2 | HNTNQEDLLV | LWGIHHPNDE | EEQTRIYKNP | NTYISVGTST | LNQRLVPKIA | TRSKVNGQSG |
| 3982-19/B3 | HNTNQEDLLV | LWGIHHPNDE | AEQXRLYKNS | TTYISVGTST | LXQRLVPKIA | TRPKVNGQSG |
| 3982-21/B2 | HNTNQEDLLV | LWGIHHPNDE | EEQTRIYKNP | TTYISVGTST | LNQRLVPKIA | TRSKVNGQSG |
| 3982-43/B1 | HNTNQEDLLV | LWGIHHPNDE | EEQKRIYKNP | TTYISVGTST | LNQRLVPKIA | TRSKVNGQSG |
| 3982-50/B1 | HNTNQEDLLV | LWGIHHPNDE | EEQTRIYKNP | TTYISVGTST | LNQRLVPKIA | TRSKVNGQSG |
| 3982-52/B1 | HNTNQEDLLV | LWGIHHPNDE | EEQKRIYKNP | TTYVSVGTST | LNQRLVPKIA | TRSKVNGQSG |
| 3982-55/A1 | NNTNQEDLLV | LWGIHHPNDA | AEQTMLYQNP | TTYVSVGTST | LNQRLVPKIA | TRSKVNGQSG |
| 3982-56/A1 | NNTNQEDLLV | LWGIHHPNDA | AEQTRLYQNP | TTYISVGTST | LNQRLVPKIA | TRSKVNGQSG |
| 3982-78/B2 | HNTNQEDLLV | LWGIHHPNDE | EEQTRIYKNP | TTYISVGTST | LNQRLVPKIA | TRSKVNGQSG |
| 4794-17/B | HNINKEDLLV | LWGIHHPNDE | EEQIRIYKNP | TTYISVGTST | LNQRLVPKIA | TRSKVNGQSG |
| 4794-18/B | HNINKEDLLV | LWGIHHPNDE | EEQIRIYKNP | TTYISVGTST | LNQRLVPKIA | TRSKVNGQSG |
| | ....\|....\| | ....\|....\| | ....\|....\| | ....\|....\| | ....\|....\| | ....\|....\| |
| | 245 | 255 | 265 | 275 | 285 | 295 |
| 1553-1/A1 | RMEFFWTILK | SNDAINFESN | GNFIAPENAY | KIVKKGDSTI | MKSELEYGNC | NTKCQTPIGA |
| 1553-15/A1 | RMEFFWTILK | SNDAINFESN | GNFIAPENAY | KIVKKGDSTI | MKSELEYGNC | NTKCQTPIGA |
| 2095-50/A1 | RMEFFWTILK | SNDAINFESN | GNFIAPENAY | KIVKKGDSTI | MKSELEYGNC | NTKCQTPIGA |
| 3982-2/A1 | RMEFFWTILK | SNDAINFESN | GNFIAPENAY | KIVKKGDSTI | MKSELEYGNC | NTKCQTPIGA |
| 3982-5/A1 | RMEFFWTILK | SNDAINFESN | GNFIAPENAY | KIVKKGDSTI | MKSELEYGNC | NTKCQTPIGA |
| 3982-7/A1 | RMEFFWTILK | SNDAINFESN | GNFIAPENAY | KIVKKGDSTI | MKSELEYGDC | NTKCQTPIGA |
| 3982-8/A1 | RMEFFWTILK | SNDAINFESN | GNFIAPENAY | KIVKKGDSTI | MKSELEYGDC | NTKCQTPIGA |
| 3982-9/A1 | RMEFFWTILK | SNDAINFESN | GNFIAPENAY | KIVKKGDSTI | MKSELEYGNC | NTKCQTPIGA |
| 3982-12/A1 | RMEFFWTILK | SNDAINFESN | GNFIAPENAY | KIVKKGDSTI | MKSELEYGNC | NTKCQTPIGA |
| 3982-20/A1 | RMEFFWTILK | SNDAINFESN | GNFIAPENAY | KIVKKGDSTI | MKSELEYGNC | NTKCQTPIGA |
| 3982-44/A1 | RMEFFWTILK | SNDAINFESN | GNFIAPENAY | KIVKKGDSTI | MKSELEYGNC | NTKCQTPIGA |
| 1553-2/B1 | RVEFFWTILK | SNDTINFESN | GNFIAPENAY | KIVKKGDSTI | MKSELEYGNC | STKCQTPIGA |
| 1553-6/B1 | RVEFFWTILK | SNDTINFESN | GNFIAPENAY | KIVKKGDSTI | MKSELEYGNC | STKCQTPVGA |
| 1553-13/B2 | RVEFFWTILK | SNDTINFESN | GNFIAPENAY | KIVKKGDSTI | MKSELEYGNC | NTKCQTPIGA |
| 1553-26/B2 | RVEFFWTILK | SNDTINFESN | GNFIAPENAY | KIVKKGDSTI | MKSELEYGNC | NTKCQTPIGA |
| 1553-28/B1 | RVEFFWTILK | SNDTINFESN | GNFIAPKNAY | KIVKKGSSTI | MKSELEYGNC | STKCQTPIGA |
| 2095-39/B2 | RVEFFWTILK | SNDTINFESN | GNFIAPENAY | KIVKKGDSTI | MKSELEYGNC | NTKCQTPIGA |
| 2095-46/B1 | RVEFFWTILK | SNDTINFESN | GNFIAPKNAY | KIVKKGSSTI | MKSELEYGNC | STKCQTPIGA |
| 2095-49/B1 | RVEFFWTILK | SNDTINFESN | GNFIAPKNAY | KIVKKGSSTI | MKSELEYGNC | STKCQTPIGA |
| 2095-65/B1 | RVEFFWTILK | SNDTINFESN | GNFIAPKNAY | KIVKKGSSTI | MKSELEYGNC | STKCQTPIGA |
| 2095-68/B2 | RVEFFWTILK | SNDTINFESN | GNFIAPENAY | KIVKKGDSTI | MKSELEHGNC | NTKCQTPIGA |
| 2095-70/B2 | RVEFFWTILK | SNDTINFESN | GNFIAPENAY | KIVKKGDSTI | MKSELEYGNC | NTKCQTPIGA |
| 2095-73/B2 | RVEFFWTILK | SNDTINFESN | GNFIAPENAY | KIVKKGDSTI | MKSELEYGNC | NTKCQTPIGA |
| 2095-75/B2 | RVEFFWTILK | SNDTINFESN | GNFIAPENAY | KIVKKGDSTI | MKSELEYGNC | NTKCQTPIGA |
| 3982-3/B1 | RVEFFWTILK | SNDTINFESN | GNFIAPKNAY | KIVKKGSSTI | MKSELEYGNC | STKCQTPIGA |
| 3982-4/B1 | RVEFFWTILK | SNDTINFESN | GNFIAPKNAY | KIVKKGSSTI | MKSELEYGNC | STKCQTPIGA |
| 3982-13/B1 | RVEFFWTILK | SNDTINFESN | GNFIAPKNAY | KIVKKGSSTI | MKSELEYGNC | STKCQTPIGA |
| 3982-14/B2 | RVEFFWTILK | SNDTINFESN | GNFIAPENAY | KIVKKGDSTI | MKSELEYGNC | STKCQTPIGA |
| 3982-19/B3 | RVEFFWTILK | SNDVINFESN | GNFIAPENAY | KIVKKGDSTI | MKSDLEYGNC | STKCQTPIGA |
| 3982-21/B2 | RVEFFWTILK | SNDTINFESN | GNFIAPENAY | KIVKKGDSTI | MKSELEYGNC | NTKCQTPIGA |
| 3982-43/B1 | RVEFFWTILK | SNDTINFESN | GNFIAPENAY | KIVKKGDSTI | MKSELEYGNC | STKCQTPIGA |
| 3982-50/B1 | RVEFFWTILK | SNDTINFESN | GNFIAPENAY | KIVKKGDSTI | MKSELEYGNC | STKCQTPVGA |
| 3982-52/B1 | RVEFFWTILK | SNDTINFESN | GNFIAPENAY | KIVKKGDSTI | MKSELEYGNC | STKCQTPIGA |
| 3982-55/A1 | RMEFFWTILK | SNDAINFESN | GNFIAPENAY | KIVKKGDSTI | MKSELEYGNC | NTKCQTPIGA |
| 3982-56/A1 | RMEFFWTILK | SNDAINFESN | GNFIAPENAY | KIVKKGDSTI | MKSELEYGNC | NTKCQTPIGA |
| 3982-78/B2 | RVEFFWTILK | SNDTINFESN | GNFIAPENAY | KIVKKGDSTI | MKSELEYGNC | NTKCQTPIGA |
| 4794-17/B | RVEFFWTILK | SNDTINFESN | GNFIAPENAY | KIVKKGDSTI | MKSELEYGNC | NTKCQTPIGA |
| 4794-18/B | RVEFFWTILK | SNDTINFESN | GNFIAPENAY | KIVKKGDSTI | MKSELEYGNC | NTKCQTPIGA |
| | ....\|....\| | ....\|....\| | ....\|....\| | ....\|....\| | ....\|....\| | ....\|....\| |
| | 305 | 315 | 325 | 335 | 345 | 355 |
| 1553-1/A1 | INSSMPFHNI | HPLTIGECPK | YVKSNRLVLA | TGLRNSPQEE | RRRKKRGLFG | AIAGFIEGGW |
| 1553-15/A1 | INSSMPFHNI | HPLTIGECPK | YVKSNRLVLA | TGLRNSPQGE | RRRKKRGLFG | AIAGFIEGGW |
| 2095-50/A1 | INSSMPFHNI | HPLTIGECPK | YVKSNRLVLA | TGLRNSPQGE | RRRKKRGLFG | AIAGFIEGGW |
| 3982-2/A1 | INSSMPFHNI | HPLTIGECPK | YVKSNRLVLA | TGLRNSPHGE | RRRKKRGLFG | AIAGFIEGGW |
| 3982-5/A1 | INSSMPFHNI | HPLTIGECPK | YVKSNRLVLA | TGLRNSPQGE | RRRKKRGLFG | AIAGFIEGGW |
| 3982-7/A1 | INSSMPFHNI | HPLTIGECPK | YVKSNRLVLA | TGLRNSPQGE | RRRKKRGLFG | AIAGFIEGGW |
| 3982-8/A1 | INSSMPFHNI | HPLTIGECPK | YVKSNRLVLA | TGLRNSPQGE | RRRKKRGLFG | AIAGFIEGGW |
| 3982-9/A1 | INSSMPFHNI | HPLTIGECPK | YVKSNRLVLA | TGLRNSPQGE | RRRKKRGLFG | AIAGFIEGGW |
| 3982-12/A1 | INSSMPFHNI | HPLTIGECPK | YVKSNRLVLA | TGLRNSPQGE | RRRKKRGLFG | AIAGFIEGGW |
| 3982-20/A1 | INSSMPFHNI | HPLTIGECPK | YVKSNRLVLA | TGLRNSPQGE | RRRKKRGLFG | AIAGFIEGGW |
| 3982-44/A1 | INSSMPFHNI | HPLTIGECPK | YVKSNRLVLA | TGLRNSPQGE | RRRKKRGLFG | AIAGFIEGGW |
| 1553-2/B1 | INTSMPFHNI | HPLTIGECPK | YVKSNRLVLA | TGLRNSPQGE | GRRKKRGLFG | AIAGFIEGGW |
| 1553-6/B1 | INSSMPFHNI | HPLTIGECPK | YVKSNRLVLA | TGLRNSPQGE | GRRKKRGLFG | AIAGFIEGGW |
| 1553-13/B2 | INSSMPFHNI | HPLTIGECPK | YVKSNRLVLA | TGLRNSPQGE | GRRKKRGLFG | AIAGFIEGGW |
| 1553-26/B2 | INSSMPFHNI | HPLTIGECPK | YVKSNRLVLA | TGLRNSPQGE | GRRKKRGLFG | AIAGFIEGGW |
| 1553-28/B1 | INSSMPFHNI | HPLTIGECPK | YVKSNRLVLA | TGLRNSPQGE | GRRKKRGLFG | AIAGFIEGGW |
| 2095-39/B2 | INSSMPFHNI | HPLTIGECPK | YVKSNRLVLA | TGLRNSPQGE | GRRKKRGLFG | AIAGFIEGGW |
| 2095-46/B1 | INSSMPFHNI | HPLTIGECPK | YVKSNRLVLA | TGLRNSPQGE | GRRKKRGLFG | AIAGFIEGGW |
| 2095-49/B1 | INSSMPFHNI | HPLTIGECPK | YVKSNRLVLA | TGLRNSPQGE | GRRKKRGLFG | AIAGFIEGGW |
| 2095-65/B1 | INSSMPFHNI | HPLTIGECPK | YVKSNRLVLA | TGLRNSPQGE | GRRKKRGLFG | AIAGFIEGGW |
| 2095-68/B2 | INSSMPFHNI | HPLTIGECPK | YVKSNRLVLA | TGLRNSPQGE | GRRKKRGLFG | AIAGFIEGGW |
| 2095-70/B2 | INSSMPFHNI | HPLTIGECPK | YVKSNRLVLA | TGLRNSPQGE | GRRKKRGLFG | AIAGFIEGGW |
| 2095-73/B2 | INSSMPFHNI | HPLTIGECPK | YVKSNRLVLA | TGLRNSPQGE | GRRKKRGLFG | AIAGFIEGGW |
| 2095-75/B2 | INSSMPFHNI | HPLTIGECPK | YVKSNRLVLA | TXLRNSPQGE | GRRKKRGLFG | AIAGFIEGGW |
| 3982-3/B1 | INSSMPFHNI | HPLTIGECPK | YVKSNRLVLA | TGLRNSPQGE | GRRKKRGLFG | AIAGFIEGGW |
| 3982-4/B1 | INSSMPFHNI | HPLTIGECPK | YVKSNRLVLA | TGLRNSPQGE | GRRKKRGLFG | AIAGFIEGGW |
| 3982-13/B1 | INSSMPFHNI | HPLTIGECPK | YVKSNRLVLA | TGLRNSPQGE | GRRKKRGLFG | AIAGFIEGGW |
| 3982-14/B2 | INSSMPFHNI | HPLTIGECPK | YVKSNRLVLA | TGLRNSPQEE | GRRKKRGLFG | AIAGFIEGGW |
| 3982-19/B3 | INSSMPFHNI | HPLTIGECPK | YVKSNRLVLA | TGLRNSPQGE | RRRKKRGLFG | AIAGFIEGGW |
| 3982-21/B2 | INSSMPFHNI | HPLTIGECPK | YVKSNRLVLA | TGLRNSPQGE | GRRKKRGLFG | AIAGFIEGGW |
| 3982-43/B1 | INTSMPFHNI | HPLTIGECPK | YVKSNRLVLA | TGLRNSPQGE | GRRKKRGLFG | AIAGFIEGGW |
| 3982-50/B1 | INSSMPFHNI | HPLTIGECPK | YVXSNRLVLA | TGLRNSPQGE | GRRKKRGLFG | AIAGFIEGGW |
| 3982-52/B1 | INTSMPFHNI | HPLTIGECPK | YVKSNRLVLA | TGLRNSPQGE | GRRKKRGLFG | AIAGFIEGGW |
| 3982-55/A1 | INSSMPFHNI | HPLTIGECPK | YVKSNRLVLA | TGLRNSPQGE | RRRKKRGLFG | AIAGFIEGGW |
| 3982-56/A1 | INSSMPFHNI | HPLTIGECPK | YVKSNRLVLA | TGLRNSPQGE | RRRKKRGLFG | AIAGFIEGGW |
| 3982-78/B2 | INSSMPFHNI | HPLTIGECPK | YVKSNRLVLA | TGLRNSPQGE | GRRKKRGLFG | AIAGFIEGGW |
| 4794-17/B | INSSMPFHNI | HPLTIGECPK | YVKSNRLVLA | TGLRNSPQGE | GRRKKRGLFG | AIAGFIEGGW |
| 4794-18/B | INSSMPFHNI | HPLTIGECPK | YVKSNRLVLA | TGLRNSPQGE | GRRKKRGLFG | AIAGFIEGGW |
| | ....\|....\| | ....\|....\| | ....\|....\| | ....\|....\| | ....\|....\| | ....\|....\| |
| | 365 | 375 | 385 | 395 | 405 | 415 |
| 1553-1/A1 | QGMVDGWYGY | HHSNEQGSGY | AADKESTQKA | IDGVTNKVNS | IIDKMNTQFE | AVGREFNNLE |
| 1553-15/A1 | QGMVDGWYGY | HHSNEQGSGY | AADKESTQKA | IDGVTNKVNS | IIDKMNTQFE | AVGREFNNLE |
| 2095-50/A1 | QGMVDGWYGY | HHSNEQGSGY | AADKESTQKA | IDGVTNKVNS | IIDKMNTQFE | AVGREFNNLE |
| 3982-2/A1 | QGMVDGWYGY | HHSNEQGSGY | AADKESTQKA | IDGVTNKVNS | IIDKMNTQFE | AVGREFNNLE |
| 3982-5/A1 | QGMVDGWYGY | HHSNEQGSGY | AADKESTQKA | IDGVTNKVNS | IIDKMNTQFE | AVGREFNNLE |
| 3982-7/A1 | QGMVDGWYGY | HHSNEQGSGY | AADKESTQKA | IDGVTNKVNS | IIDKMNTQFE | AVGREFNNLE |
| 3982-8/A1 | QGMVDGWYGY | HHSNEQGSGY | AADKESTQKA | IDGVTNKVNS | IIDKMNTQFE | AVGREFNNLE |
| 3982-9/A1 | QGMVDGWYGY | HHSNEQGSGY | AADKESTQKA | IDGVTNKVNS | IIDKMNTQFE | AVGREFNNLE |
| 3982-12/A1 | QGMVDGWYGY | HHSNEQGSGY | AADKESTQKA | IDGVTNKVNS | IIDKMNTQFE | AVGREFNNLE |
| 3982-20/A1 | QGMVDGWYGY | HHSNEQGSGY | AADKESTQKA | IDGVTNKVNS | IIDKMNTQFE | AVGREFNNLE |
| 3982-44/A1 | QGMVDGWYGY | HHSNEQGSGY | AADKESTQKA | IDGVTNKVNS | IIDKMNTQFE | AVGREFNNLE |
| 1553-2/B1 | QGMVDGWYGY | HHSNEQGSGY | AADKESTQKA | IDGVTNKVNS | IIDKMNTQFE | AVGREFNNLE |
| 1553-6/B1 | QGMVDGWYGY | HHSNXQGSGY | AADRESTQKA | IDGVTNKVNS | IIDKMNTQFE | AVGREFNNLE |
| 1553-13/B2 | QGMVDGWYGY | HHSNEQGSGY | AADKESTQKA | IDGVTNKVNS | IIDKMNTQFE | AVGREFNNLE |
| 1553-26/B2 | QGMVDGWYGY | HHSNEQGSGY | AADKESTQKA | IDGVTNKVNS | IIDKMNTQFE | AVGREFNNLE |
| 1553-28/B1 | QGMVDGWYGY | HHSNEQGTGY | AADKESTQKA | IDGVTNKVNS | IIDKMNTQFE | AVGREFNNLE |
| 2095-39/B2 | QGMVDGWYGY | HHSNEQGSGY | AADKESTQKA | IDGVTNKVNS | IIDKMNTQFE | AVGREFNNLE |
| 2095-46/B1 | QGMVDGWYGY | HHSNEQGTGY | AADKESTQKA | IDGVTNKVNS | IIDKMNTQFE | AVGREFNNLE |
| 2095-49/B1 | QGMVDGWYGY | HHSNEQGTGY | AADKESTQKA | IDGVTNKVNS | IIDKMNTQFE | AVGREFNNLE |
| 2095-65/B1 | QGMVDGWYGY | HHSNEQGTGY | AADKESTQKA | IDGVTNKVNS | IIDKMNTQFE | AVGREFNNLE |
| 2095-68/B2 | QGMVDGWYGY | HHSNEQGSGY | AADKESTQKA | IDGVTNKVNS | IIDKMNTQFE | AVGREFNNLE |
| 2095-70/B2 | QGMVDGWYGY | HHSNEQGSGY | AADKESTQKA | IDGVTNKVNS | IIDKMNTQFE | AVGREFNNLE |
| 2095-73/B2 | QGMVDGWYGY | HHSNEQGSGY | AADKESTQKA | IDGVTNKVNS | IIDKMNTQFE | AVGREFNNLE |
| 2095-75/B2 | QGMVDGWYGY | HHSNEQGSGY | AADKESTQKA | IDGVTNKVNS | IIDKMNTQFE | AVGREFNNLE |
| 3982-3/B1 | QGMVDGWYGY | HHSNEQGTGY | AADKESTQKA | IDGVTNKVNS | IIDKMNTQFE | AVGREFNNLE |
| 3982-4/B1 | QGMVDGWYGY | HHSNEQGTGY | AADKESTQKA | IDGVTNKVNS | IIDKMNTQFE | AVGREFNNLE |
| 3982-13/B1 | QGMVDGWYGY | HHSNEQGTGY | AADKESTQKA | IDGVTNKVNS | IIDKMNTQFE | AVGREFNNLE |
| 3982-14/B2 | QGMVDGWYGY | HHSNEQGSGY | AADKESTQKA | IDGVTNKVNS | IIDKMNTQFE | AVGREFNNLE |
| 3982-19/B3 | QGMVDGWYGY | HHSNEQGSGY | AADKESTQKA | IDGVTNKVNS | IIDKMNTQFE | AVGREFNNLE |
| 3982-21/B2 | QGMVDGWYGY | HHSNEQGSGY | AADKESTQKA | IDGVTNKVNS | IIDKMNTQFE | AVGREFNNLE |
| 3982-43/B1 | QGMVDGWYGY | HHSNEQGSGY | AADKESTQKA | INGVTNKVNS | IIDKMNTQFE | AVGREFNNLE |
| 3982-50/B1 | QGMVDGWYGY | HHSNEQGSGY | AADRESTQKA | IDGVTNKVNS | IIDKMNTQFE | AVGREFNNLE |
| 3982-52/B1 | QGMVDGWYGY | HHSNEQGSGY | AADKESTQKA | IDGVTNKVNS | IIDKMNTQFE | AVGREFNNLE |
| 3982-55/A1 | QGMVDGWYGY | HHSNEQGSGY | AADKESTQKA | IDGVTNKVNS | IIDKMNTQFE | AVGREFNNLE |
| 3982-56/A1 | QGMVDGWYGY | HHSNEQGSGY | AADKESTQKA | IDGVTNKVNS | IIDKMNTQFE | AVGREFNNLE |
| 3982-78/B2 | QGMVDGWYGY | HHSNEQGSGY | AADKESTQKA | IDGVTNKVNS | IIDKMNTQFE | AVGREFNNLE |
| 4794-17/B | QGMVDGWYGY | HHSNEQGSGY | AADKESTQKA | IDGVTNKVNS | IIDKMNTQFE | AVGREFNNLE |
| 4794-18/B | QGMVDGWYGY | HHSNEQGSGY | AADKESTQKA | IDGVTNKVNS | IIDKMNTQFE | AVGREFNNLE |
| | ....\|....\| | ....\|....\| | ....\|....\| | ....\|....\| | ....\|....\| | ....\|....\| |
| | 425 | 435 | 445 | 455 | 465 | 475 |
| 1553-1/A1 | RRIENLNKKM | EDGFLDVWTY | NAELLVLMEN | ERTLDFHDSN | VKNLYDKVRL | QLRDNAKELG |
| 1553-15/A1 | RRIENLNKKM | EDGFLDVWTY | NAELLVLMEN | ERTLDFHDSN | VKNLYDRVRL | QLRDNAKELG |
| 2095-50/A1 | RRIENLNKKM | EDGFLDVWTY | NAELLVLMEN | ERTLDFHDSN | VKNLYDKVRL | QLRDNAKELG |
| 3982-2/A1 | RRIENLNKKM | EDGFLDVWTY | NAELLVLMEN | ERTLDFHDSN | VKNLYDKVRL | QLRDNAKELG |
| 3982-5/A1 | RRIENLNKKM | EDGFLDVWTY | NAELLVLMEN | ERTLDFHDSN | VKNLYDKVRL | QLRDNAKELG |
| 3982-7/A1 | RRIENLNKKM | EDGFLDVWTY | NAELLVLMEN | ERTLDFHDSN | VKNLYDKVRL | QLRDNAKELG |
| 3982-8/A1 | RRIENLNKKM | EDGFLDVWTY | NAELLVLMEN | ERTLDFHDSN | VKNLYDKVRL | QLRDNAKELG |
| 3982-9/A1 | RRIENLNKKM | EDGFLDVWTY | NAELLVLMEN | ERTLDFHDSN | VKNLYDKVRL | QLRDNAKELG |
| 3982-12/A1 | RRIENLNKKM | EDGFLDVWTY | NAELLVLMEN | ERTLDFHDSN | VKNLYDKVRL | QLRDNAKELG |
| 3982-20/A1 | RRIENLNKKM | EDGFLDVWTY | NAELLVLMEN | ERTLDFHDSN | VKNLYDKVRL | QLRDNAKELG |
| 3982-44/A1 | RRIENLNKKM | EDGFLDVWTY | NAELLVLMEN | ERTLDFHDSN | VKNLYDKVRL | QLRDNAKELG |
| 1553-2/B1 | KRIENLNKKM | EDGFLDVWTY | NAELLVLMEN | ERTLDFHDSN | VRNLYDKVRL | QLRDNAKELG |
| 1553-6/B1 | KRIENLNKKM | EDGFLDVWTY | NAELLVLMEN | ERTLDFHDSN | VKNLYDKVRL | QLRDNAKELG |
| 1553-13/B2 | KRIENLNKKM | EDGFLDVWTY | NAELLVLMEN | ERTLDFHDSN | VKNLYDKVRL | QLRDNAKELG |
| 1553-26/B2 | KRIENLNKKM | EDGFLDVWTY | NAELLVLMEN | ERTLDFHDSN | VKNLYDKVRL | QLRDNAKELG |
| 1553-28/B1 | KRIENLNKKM | EDGFLDVWTY | NAELLVLMEN | ERTLDFHDSN | VRNLYDKVRL | QLRDNAKELG |
| 2095-39/B2 | KRIENLNKKM | EDGFLDVWTY | NAELLVLMEN | ERTLDFHDSN | VKNLYDKVRL | QLRDNAKELG |
| 2095-46/B1 | KRIENLNKKM | EDGFLDVWTY | NAELLVLMEN | ERTLDFHDSN | VRNLYDKVRL | QLRDNAKELG |
| 2095-49/B1 | KRIENLNKKM | EDGFLDVWTY | NAELLVLMEN | ERTLDFHDSN | VRNLYDKVRL | QLRDNAKELG |
| 2095-65/B1 | KRIENLNKKM | EDGFLDVWTY | NAELLVLMEN | ERTLDFHDSN | VRNLYDKVRL | QLRDNAKELG |
| 2095-68/B2 | KRIENLNKKM | EDGFLDVWTY | NAELLVLMEN | ERTLDFHDSN | VKNLYDKVRL | QLRDNAKELG |
| 2095-70/B2 | KRIENLNKKM | EDGFLDVWTY | NAELLVLMEN | ERTLDFHDSN | VKNLYDKVRL | QLRDNAKELG |
| 2095-73/B2 | KRIENLNKKM | EDGFLDVWTY | NAELLVLMEN | ERTLDFHDSN | VKNLYDKVRL | QLRDNAKELG |
| 2095-75/B2 | KRIENLNKKM | EDGFLDVWTY | NAELLVLMEN | ERTLDFHDSN | VKNLYDKVRL | QLRDNAKELG |
| 3982-3/B1 | KRIENLNKKM | EDGFLDVWTY | NAELLVLMEN | ERTLDFHDSN | VRNLYDKVRL | QLRDNAKELG |
| 3982-4/B1 | KRIENLNKKM | EDGFLDVWTY | NAELLVLMEN | ERTLDFHDSN | VRNLYDKVRL | QLRDNAKELG |
| 3982-13/B1 | KRIENLNKKM | EDGFLDVWTY | NAELLVLMEN | ERTLDFHDSN | VRNLYDKVRL | QLRDNAKELG |
| 3982-14/B2 | KRIENLNKKM | EDGFLDVWTY | NAELLVLMEN | ERTLDFHDSN | VKNLYDKVRL | QLRDNAKELG |
| 3982-19/B3 | RRIENLNKKM | EDGFLDVWTY | NAELLVLMEN | ERTLDFHDSN | VKNLYDKVRL | QLRDNAKELG |
| 3982-21/B2 | KRIENLNKKM | EDGFLDVWTY | NAELLVLMEN | ERTLDFHDSN | VKNLYDKVRL | QLRDNAKELG |
| 3982-43/B1 | KRIENLNKKM | EDGFLDVWTY | NAELLVLMEN | ERTLDFHDSN | VRNLYDKVRL | QLRDNAKELG |
| 3982-50/B1 | KRIENLNKKM | EDGFLDVWTY | NAELLVLMEN | ERTLDFHDSN | VKNLYDKVRL | QLRDNAKELG |
| 3982-52/B1 | KRIENLNKKM | EDGFLDVWTY | NAELLVLMEN | ERTLDFHDSN | VRNLYDKVRL | QLRDNAKELG |
| 3982-55/A1 | RRIENLNKKM | EDGFLDVWTY | NAELLVLMEN | ERTLDFHDSN | VKNLYDKVRL | QLRDNAKELG |
| 3982-56/A1 | RRIENLNKKM | EDGFLDVWTY | NAELLVLMEN | ERTLDFHDSN | VKNLYDKVRL | QLRDNAKELG |
| 3982-78/B2 | KRIENLNKKM | EDGFLDVWTY | NAELLVLMEN | ERTLDFHDSN | VKNLYDKVRL | QLRDNAKELG |
| 4794-17/B | KRIENLNKKM | EDGFLDVWTY | NAELLVLMEN | ERTLDFHDSN | VKNLYDKVRL | QLRDNAKELG |
| 4794-18/B | KRIENLNKKM | EDGFLDVWTY | NAELLVLMEN | ERTLDFHDSN | VKNLYDKVRL | QLRDNAKELG |
| | ....\|....\| | ....\|....\| | ....\|....\| | ....\|....\| | ....\|....\| | ....\|....\| |
| | 485 | 495 | 505 | 515 | 525 | 535 |
| 1553-1/A1 | NGCFEFYHRC | DNECMESVRN | GTYDYPQYSE | EARLKREEIS | GVKLESIGTY | QILSIYSTVA |
| 1553-15/A1 | NGCFEFYHRC | DNECMESVRN | GTYDYPQYSE | EARLKREEIS | GVKLESIGTY | QILSIYSTVA |
| 2095-50/A1 | NGCFEFYHRC | DNECMESVRN | GTYDYPQYSE | EARLKREEIS | GVKLESIGTY | QILSIYSTVA |
| 3982-2/A1 | NGCFEFYHRC | DNECMESVRN | GTYDYPQYSE | EARLKREEIS | GVKLESIGTY | QILSIYSTVA |
| 3982-5/A1 | NGCFEFYHRC | DNECMESVRN | GTYDYPQYSE | EARLKREEIS | GVKLESIGTY | QILSIYSTVA |
| 3982-7/A1 | NGCFEFYHRC | DNECMESVRN | GTYDYPQYSE | EARLKREEIS | GVKLESIGTY | QILSIYSTVA |
| 3982-8/A1 | NGCFEFYHRC | DNECMESVRN | GTYDYPQYSE | EARLKREEIS | GVKLESIGTY | QILSIYSTVA |
| 3982-9/A1 | NGCFEFYHRC | DNECMESVRN | GTYDYPQYSE | EARLKREEIS | GVKLESIGTY | QILSIYSTVA |
| 3982-12/A1 | NGCFEFYHRC | DNECMESVRN | GTYDYPQYSE | EARLKREEIS | GVKLESIGTY | QILSIYSTVA |
| 3982-20/A1 | NGCFEFYHRC | DNECMESVRN | GTYDYPQYSE | EARLKREEIS | GVKLESIGTY | QILSIYXTVA |
| 3982-44/A1 | NGCFEFYHRC | DNECMESVRN | GTYDYPQYSE | EARLKREEIS | GVKLESIGTY | QILSIYSTVA |
| 1553-2/B1 | NGCFEFYHRC | DNECMESVRN | GTYDYPQYSE | EARLKREEIS | GVKLESIGTY | QILSIYSTVA |
| 1553-6/B1 | NGCFEFYHRC | DNECIESVRN | GTYDYPQYSE | EARLKREEIS | GVKLESIGTY | QILSIYSTVA |
| 1553-13/B2 | NGCFEFYHRC | DNECMESVRN | GTYDYPQYSE | EARLKREEIS | GVKLESIGTY | QILSIYSTVA |
| 1553-26/B2 | NGCFEFYHRC | DNECMESVRN | GTYDYPQYSE | EARLKREEIS | GVKLESIGTY | QILSIYSTVA |
| 1553-28/B1 | NGCFEFYHKC | DNECMESVRN | GTYDYPQYSK | EARLKREEIS | GVKLESIGTY | QILSIYSTVT |
| 2095-39/B2 | NGCFEFYHRC | DNECMESVRN | GTYDYPQYSE | EARLKREEIS | GVKLESIGTY | QILSIYSTVA |
| 2095-46/B1 | NGCFEFYHKC | DNECMESVRN | GTYDYPQYSK | EARLKREEIS | GVKLESIGTY | QILSIYSTVA |
| 2095-49/B1 | NGCFEFYHKC | DNECMESVRN | GTYDYPQYSK | EARLKREEIS | GVKLESIGTY | QILSIYSTVA |
| 2095-65/B1 | NGCFEFYHKC | DNECMESVRN | GTYDYPQYSK | EARLKREEIS | GVKLESIGTY | QILSIYSTVA |
| 2095-68/B2 | NGCFEFYHRC | DNECMESVRN | GTYDYPQYSE | EARLNREEIS | GVKLESIGTY | QILSIYSTVA |
| 2095-70/B2 | NGCFEFYHRC | DNECMESVRN | GTYDYPQYSE | EARLKREEIS | GVKLESIGTY | QILSIYSTVA |
| 2095-73/B2 | NGCFEFYHRC | DNECMESVRN | GTYDYPQYSE | EARLKREEIS | GVKLESIGTY | QILSIYSTVA |
| 2095-75/B2 | NGCFEFYHRC | DNECMESVRN | GTYDYPQYSE | EARLKREEIS | GVKLESIGTY | QILSIYSTVA |
| 3982-3/B1 | NGCFEFYHKC | DNECMESVRN | GTYDYPQYSK | EARLKREEIS | GVKLESIGTY | QILSIYSTVA |
| 3982-4/B1 | NGCFEFYHKC | DNECMESVRN | GTYDYPQYSK | EARLKREEIS | GVKLESIGTY | QILSIYSTVA |
| 3982-13/B1 | NGCFEFYHKC | DNECMESVRN | GTYDYPQYSK | EARLKREEIS | GVKLESIGTY | QILSIYSTVA |
| 3982-14/B2 | NGCFEFYHRC | DNECMESVRN | GTYDYPQYSE | EARLKREEIS | GVKLESIGTY | QILSIYSTVA |
| 3982-19/B3 | NGCFEFYHRC | DNECMESVRN | GTYDYPQYSE | EARLKREEIS | GAKLESIGTY | QIXSIYSTVA |
| 3982-21/B2 | NGCFEFYHRC | DNECMESVKN | GTYDYPQYSE | EARLKREEIS | GVKLESIGTY | QILSIYSTVA |
| 3982-43/B1 | NGCFEFYHRC | DNECMESVRN | GTYDYPQYSK | EARLKREEIS | GVKLESIGTY | QILSIYSTVA |
| 3982-50/B1 | NGCFEFYHRC | DNECIESVRN | GTYDYPQYSE | EARLKREEIS | GVKLESIGTY | QILSIYSTVA |
| 3982-52/B1 | NGCFEFYHRC | DNECMESVRN | GTYDYPQYSE | EARLKREEIS | GVKLEXIGTY | QILSIYSTVA |
| 3982-55/A1 | NGCFEFYHRC | DNECMESVRN | GTYDYPQYSE | EARLKREEIS | GVKLESIGTX | QILSIYSTVA |
| 3982-56/A1 | NGCFEFYHRC | DNECMESVRN | GTYDYPQYSE | EARLKREEIS | GVKLESIGTX | QIXSIYSTVA |
| 3982-78/B2 | NGCFEFYHRC | DNECMESVRN | GTYDYPQYSE | EARLKREEIS | GVKLESIGTY | QIL*IYSTVA |
| 4794-17/B | NGCFEFYHRC | DNECMESVRN | GTYDYPQYSE | EARLKREEIS | GVKLESIGTY | QILSIYSTVA |
| 4794-18/B | NGCFEFYHRC | DNECMESVRN | GTYDYPQYSE | EARLKREEIS | GVKLESIGTY | QILSIYSTVA |
| | ....\|....\| | ....\|....\| | ....\|.... | | | |
| | 545 | 555 | 565 | | | |
| 1553-1/A1 | SSLALAIMVA | GLFLWMCSNG | SLQCRICI | | | |
| 1553-15/A1 | SSLALAIMVA | GLFLWMCSNG | SLQCRICI | | | |
| 2095-50/A1 | SSLALAIMVA | GLFLWMCSNG | XLQCRICI | | | |
| 3982-2/A1 | SSLALAIMVX | GLFLWMCSNG | SLQCRICI | | | |
| 3982-5/A1 | SSLALAIMVA | GLFLWMCSNG | SLQCRICI | | | |
| 3982-7/A1 | SSLALAIMVA | GLFLWMCSNG | XLQCRICI | | | |
| 3982-8/A1 | SSLALAIMVX | GLFLWMCSNG | SLQCRICI | | | |
| 3982-9/A1 | SSLALAIMVA | GLFLWMCSNG | SLQCRICI | | | |
| 3982-12/A1 | SSLALAIXVA | GLFLWMCSNG | SLQCRICI | | | |
| 3982-20/A1 | SSLALAIMVA | GLFLWMCSNG | SLQCRICI | | | |
| 3982-44/A1 | SSLALAIXVA | GLFLWMCSNG | SLQCRICI | | | |
| 1553-2/B1 | SSLALAIMVA | GLFLWMCSNG | SLQCRICI | | | |
| 1553-6/B1 | SSLALAIIVA | GLFLWMCSNG | SLQCRICI | | | |
| 1553-13/B2 | SSLALAIMMA | GLFLWMCSNG | SLQCRICI | | | |
| 1553-26/B2 | SSLALAIMMA | GLFLWMCSNG | SLQCRICI | | | |
| 1553-28/B1 | SSLALAIMVA | GLSLWMCSNG | SLQCRICI | | | |

TABLE D
The sequence SEQ ID NO: 138 is shown, which correspond to the
variant sequence as set forth in SEQ ID NO: 47 starting with the N-
terminal amino acid D17, wherein the numbering of said first N-
terminal amino acid position (D17) refers to the amino acid position
17 as exemplarily given in SEQ ID NO 8:

```
                10 20 30 40 50 60
(SEQ ID NO: 47) DQIC IGYHANNSTE QVDTIMEKNV TVTHAQDILE KTHNGKLCDL
                70 80 90 100 110 120
DGVKPLILRD CSVAGWLLGN PMCDEFPNVS EWSYIVEKIN PANDLCYPGN FNNYEELKHL
              130 140 150 160 170 180
LSRINRFEKI QIIPKSSWPD HEASLGVSSA CPYQGGPSFY RNVVWLIKKN NTYPTIKESY
              190 200 210 220 230 240
HNTNQEDLLV LWGIHHPNDE EEQTRIYKNP TTYISVGTST LNQRLVPKIA TRSKVNGQSG
              250 260 270 280 290 300
RVEFFWTILK SNDTINFESN GNFIAPENAY KIVKKGDSTI MKSELEYGNC NTKCQTPIGA
              310 320 330 340 350 360
INSSMPFHNI HPLTIGECPK YVKSNRLVLA TGLRNSPQGE GRRKKRGLFG AIAGFIEGGW
              370 380 390 400 410 420
QGMVDGWYGY HHSNEQGSGY AADKESTQKA IDGVTNKVNS IIDKMNTQFE AVGREFNNLE
              430 440 450 460 470 480
KRIENLNKKM EDGFLDVWTY NAELLVLMEN ERTLDFHDSN VKNLYDKVRL QLRDNAKELG
              490 500 510 520 530 540
NGCFEFYHRC DNECMESVRN GTYDYPQYSE EARLKREEIS GVKLESIGTY QILSIYSTVA
              550 560
SSLALAIMVA GLFLWMCSNG SLQCRICI
```

It is understood that the sequence provided in Table D (SEQ ID NO: 138) is a variant of the sequence SEQ ID NO: 47 starting with amino acid D17 and having the full length C-terminal sequence, as mentioned herein.

1. A method of treating or preventing infections with H5N1 virus of a different clade comprising administering to a subject an H5 protein of clade 1 H5N1 virus, wherein said H5 protein comprises a polypeptide sequence having at least 98% sequence identity with the polypeptide sequence of SEQ ID NO:1, wherein said H5N1 virus of a different clade is a H5N1 virus comprising:
a. a polynucleotide encoding a second H5 protein of influenza virus, wherein said second H5 protein encodes an amino acid sequence comprising:
i. the amino acids 113D, 126H, 145(−), 156R, 160F, 167T, and 181N, wherein the modification 145(−) means that amino acid position 145 of H5 is deleted, or
ii. the amino acids 87P, 145L, 172T, 201E, 206I, 208K, 254T, 341G and 421K, or
iii. the amino acids 145L, 172T, and 254V,
wherein the numbering of the amino acid positions of the second H5 protein of group (i), (ii), and (iii) refers to the amino acid position as exemplarily given in SEQ ID NO:8, or
iv. an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46; or
b. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137.

2. The method according to claim 1, wherein said H5 protein comprises a polypeptide sequence having at least 98.1%, preferably at least 98.2%, more preferably at least 98.3%, and most preferably at least 98.4% sequence identity with the polypeptide sequence of SEQ ID NO:1.

3. The method according to claim 1, wherein said H5 protein has the amino acid 223N and the modification 328K+, wherein numbering of the amino acid positions of the H5 protein refers to the amino acid position as exemplarily given in SEQ ID NO:2, and wherein the modification 328K+ means that at amino acid position 328 of the H5 protein a second Lysine (K+) is inserted.

4. The method according to claim 1, wherein such H5 protein has the amino acid 94N.

5. The method according to claim 1, wherein such H5 protein has the amino acid 120N.

6. The method according to claim 1, wherein such H5 protein has the amino acid 155N.

7. The method according to claim 1, wherein such H5 protein has one or more of the following amino acid clusters selected from the group consisting of:
a. aa 93-95: GNF
b. aa 123-125: SDH
c. aa 128-130: SSG
d. aa 138-140: GSS
e. aa 226-228: MDF
f. aa 270-272: EVE
g. aa 309-311: NKL.

8. The method according to claim 1, wherein such H5 protein comprises a peptide comprising:
a. the amino acid sequences of SEQ ID NO:5; SEQ ID NO:6 or SEQ ID NO:7; or
b. any peptide that has at least 85% sequence homology to the polypeptide of i) and that comprises hemagglutinin inhibition in a standard hemagglutinin inhibition assay; or
c. any part of the polypeptides of i) or ii) comprising at least 8 contiguous amino acids of any of such peptides of i) or ii) and wherein any of such peptide comprises hemagglutinin inhibition in a standard hemagglutinin inhibition assay; or
d. any peptide of i), ii) or iii) having one of the amino acids 36T, 36K, 83A, 83T, 83D, 86A, 86V, 120S, 155S, 156A, 156T, 189R, 189K, 212K, 212R, 212E, 263A or 263T; or
e. any peptide of i), ii), iii) or iv) having one or more of the following amino acid clusters selected from the group consisting of:
i. aa 93-95: GNF
ii. aa 123-125: SDH
iii. aa 128-130: SSG
iv. aa 138-140: GSS
v. aa 226-228: MDF
vi. aa 270-272: EVE
vii. aa 309-311: NKL.

9. The method according to claim 1, wherein such H5 protein comprises the amino acid sequence of SEQ ID NO:5.

10. The method according to claim 1, wherein such H5 protein is recombinantly expressed and/or produced by a baculovirus expression system, preferably in cultured insect cells.

11. The method according to claim 1, wherein said H5N1 virus of a different clade is a clade 2.2.1 H5N1 virus.

12. The method according to claim 1, wherein said H5N1 virus of a different clade is a H5N1 virus of North African origin.

13. A method of treating or preventing infections with H5N1 virus of a different clade comprising administering to a subject an H5 protein of clade 1 H5N1 virus, wherein said H5 protein comprises a polypeptide sequence having at least 98% sequence identity with the polypeptide sequence of SEQ ID NO:1, wherein said H5N1 virus of a different clade is a H5N1 virus comprising:
a. a polynucleotide encoding a second H5 protein comprising:
i. the amino acids 87L, 113D, 126H, 145(−), 156R, 160F, 167T, and 181N, or
ii. the amino acids 87P, 113N, 126R, 145L, 160Y, 172T, 181H, 201E, 206I, 208K, 254T, 341G and 421K, or
iii. the amino acids 87L, 113N, 126R, 145L, 156G, 160Y, 172T, 181H, and 254V,
wherein the numbering of the amino acid positions of the second H5 protein of group (i), (ii), and (iii) refers to the amino acid position as exemplarily given in SEQ ID NO:8; or
iv. any one of the amino acid sequences of SEQ ID NOs: 62 to 137; or
b. a second H5 protein comprising any peptide that has at least 85%, preferably at least 95%, even more preferably at least 96%, even more preferably at least 97%, even more preferably at least 98%, even more preferably at least 99%, most preferably 100% sequence homology to the polypeptide of i) and that comprises hemagglutinin inhibition in a standard hemagglutinin inhibition assay; or
c. a second H5 protein comprising any part of the polypeptides of a.i) or a.ii) comprising at least 334 contiguous amino acids of any of such peptides of a.i) or a.ii) and wherein any of such peptide comprises hemagglutinin inhibition in a standard hemagglutinin inhibition assay; or
d. a second H5 protein of influenza virus, wherein said second H5 protein comprises a contiguous amino acid sequence which has at least 95% even more preferably at least 96%, even more preferably at least 97%, even more preferably at least 98%, even more preferably at least 99%, most preferably 100% sequence identity with any one of the sequences as set forth in SEQ ID NOs: 62 to 137.

14. The method according to claim 13, wherein said H5N1 virus of a different clade comprising a second H5 protein comprises:
a. the amino acids 87L, 113D, 126H, 145(−), 156R, 160F, 167T, and 181N, or
b. the amino acids 87P, 113N, 126R, 145L, 160Y, 172T, 181H, 201E, 206I, 208K, 254T, 341G and 421K, or
c. the amino acids 87L, 113N, 126R, 145L, 156G, 160Y, 172T, 181H, and 254V; or
d. a peptide comprising:
i. any one of the amino acid sequences of SEQ ID NOs: 62 to 137, or
ii. any peptide that has at least 85%, preferably at least 95%, even more preferably at least 96%, even more preferably at least 97%, even more preferably at least 98%, even more preferably at least 99%, most preferably 100% sequence homology to the polypeptide of i) and that comprises hemagglutinin inhibition in a standard hemagglutinin inhibition assay, or
iii. any part of the polypeptides of i) or ii) comprising at least 334 contiguous amino acids of any of such peptides of i) or ii) and wherein any of such peptide comprises hemagglutinin inhibition in a standard hemagglutinin inhibition assay; or
e. a contiguous amino acid sequence which has at least 95% even more preferably at least 96%, even more preferably at least 97%, even more preferably at least 98%, even more preferably at least 99%, most preferably 100% sequence identity with any one of the sequences as set forth in SEQ ID NOs: 62 to 137.

15. The method according to claim 13, wherein said H5N1 virus of a different clade is a H5N1 virus comprising:
a. a polynucleotide encoding a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 99; or
b. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 99.

16. The method according to claim 13, wherein said H5N1 virus of a different clade comprises:
a. a polynucleotide encoding a second H5 protein comprising an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 68, 73, 106, or 111, and wherein said second H5 protein comprising the amino acid sequence set forth in any of SEQ ID NOs: 73 or 111 are in particular more preferred; or
b. a second H5 protein comprising an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 68, 73, 106, or 111,
wherein such second H5 protein comprising the amino acid sequence set forth in any of SEQ ID NOs:73 or 111 are in particular more preferred.

17. The method according to claim 1, for use in treating or preventing infections, wherein said infections comprise:
a. a Subclade A H5N1 virus of North African origin, namely an infection with a H5N1 virus comprising a polynucleotide encoding a second H5 protein, wherein
i. said second H5 protein comprises the amino acids according to (a.i.) of claim 1, or
ii. said second H5 protein comprises any one of the sequences as set forth in SEQ ID NOs: 62 to 72, 100 to 110, 9 to 19, or 95 or 96, 133 or 134, 42 or 43; or
b. a Subclade B H5N1 virus of North African origin, namely an infection with a H5N1 virus comprising a polynucleotide encoding a second H5 protein, wherein
i. said second H5 protein comprises the amino acids according to (a.ii) or (a.iii) of claim 1, or
ii. said second H5 protein comprises any one of the sequences as set forth in SEQ ID NOs: 82 to 94, 111 to 132, 20 to 41, or 97 to 99, 135 to 137, or 44 to 46.

18. The method according claim 1, for use treating or preventing infections, wherein said infections comprise:
a. a Subclade A H5N1 virus of North African origin, namely an infection with a H5N1 virus comprising a second H5 protein wherein i. said second H5 protein comprises the amino acids according to
(a.i) of claim 1, or
ii. said second H5 protein comprises any one of the sequences as set forth in SEQ ID NOs: 62 to 72, 100 to 110, or 95 or 96, 133 or 134; or
b. a Subclade B H5N1 virus of North African origin, namely an infection with a H5N1 virus comprising a second H5 protein wherein
i. said second H5 protein comprises the amino acids according to (a.ii) or (a.iii) of claim 1, or
ii. said second H5 protein comprises any one of the sequences as set forth in SEQ ID NOs: 82 to 94, 111 to 132, or 97 to 99, 135 to 137.

19. A method of treating or preventing infections with H5N1 virus of a different clade comprising administering to a subject a combination of a H5 protein and an inactivated Newcastle disease virus, wherein said H5 protein comprises a polypeptide sequence having at least 98% sequence identity with the polypeptide sequence of SEQ ID NO:1.

20. The method of claim 19, wherein the inactivated Newcastle disease virus is an inactivated whole Newcastle disease virion.

21. The method of claim 19, wherein the inactivated Newcastle disease virus is an inactivated Newcastle disease virus obtained by inactivation of a Newcastle disease virus comprising a RNA polynucleotide having at least 70%, preferably at least 80%, more preferably at least 90%, still more preferably at least 95% or in particular 100% sequence identity with a RNA copy of the polynucleotide set forth in SEQ ID NO: 139, which has been inactivated.

22. The method of claim 19, wherein the Newcastle disease virus is a Newcastle disease LaSota strain virus.

23. The method of claim 19, wherein the Newcastle Disease Virus is inactivated with a reagent selected from the group consisting of Formaldehyde, BEI, Beta-Propio-Lactone (BPL), and combinations thereof.

24. A method of treating or preventing infections with H5N1 virus of a clade other than clade 1, comprising administration to a subject a vaccine comprising:
a. an H5 protein comprising a polypeptide sequence having at least 98% sequence identity with the polypeptide sequence of SEQ ID NO:1, and
b. a pharmaceutical acceptable carrier and/or excipient.

25. The method according to claim 24, wherein the excipient is one or more adjuvants.

26. The method according to claim 25, wherein the adjuvant is an Emulsigen-based adjuvant.

27. The method according to claim 24, wherein the vaccine comprises one or more further antigens.

28. The method according to claim 27, wherein the one or more further antigen is an antigen of a poultry pathogen.

29. The method according to claim 28, wherein the one or more further antigen is H5, H7, or H9 of influenza virus.

30. The method according to claim 29, wherein said H5 of influenza virus is H5 protein of a H5N1 virus of a clade different than clade 1.

31. A method for the prophylaxis or treatment of infections caused by H5N1 virus of a clade other than clade 1 comprising administering to a subject a pharmaceutical composition, preferably of a single-shot vaccine or a one dose vaccine comprising an H5 protein comprising a polypeptide sequence having at least 98% sequence identity with the polypeptide sequence of SEQ ID NO:1, wherein said H5N1 virus of a different clade is a H5N1 virus comprising:
a. a polynucleotide encoding a second H5 protein of influenza virus, wherein said second H5 protein encodes an amino acid sequence comprising:
i. the amino acids 113D, 126H, 145(−), 156R, 160F, 167T, and 181N, wherein the modification 145(−) means that amino acid position 145 of H5 is deleted, or
ii. the amino acids 87P, 145L, 172T, 201E, 206I, 208K, 254T, 341G and 421K, or
iii. the amino acids 145L, 172T, and 254V, wherein the numbering of the amino acid positions of the second H5 protein of group (i), (ii), and (iii) refers to the amino acid position as exemplarily given in SEQ ID NO:8; or
iv. an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46; or
b. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137.

32. A method for the treatment or prophylaxis of influenza virus infections caused by H5N1 virus of a clade other than clade 1, comprising administration of a therapeutically effective amount of the H5 protein comprising a polypeptide sequence having at least 98% sequence identity with the polypeptide sequence of SEQ ID NO:1 to a subject in need of such a treatment, wherein said H5N1 virus of a clade other than clade 1 is a H5N1 virus comprising:
a. a polynucleotide encoding a second H5 protein of influenza virus, wherein said second H5 protein encodes an amino acid sequence comprising:
i. the amino acids 113D, 126H, 145(−), 156R, 160F, 167T, and 181N, wherein the modification 145(−) means that amino acid position 145 of H5 is deleted, or
ii. the amino acids 87P, 145L, 172T, 201E, 206I, 208K, 254T, 341G and 421K, or
iii. the amino acids 145L, 172T, and 254V,
wherein the numbering of the amino acid positions of the second H5 protein of group (i), (ii), and (iii) refers to the amino acid position as exemplarily given in SEQ ID NO:8, or
iv. an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46; or
b. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137.

33. A method for the treatment or prophylaxis of influenza virus infections caused by H5N1 virus of a clade other than clade 1, comprising administration of a therapeutically effective amount of a vaccine comprising an H5 protein comprising a polypeptide sequence having at least 98% sequence identity with the polypeptide sequence of SEQ ID NO:1, and a pharmaceutical acceptable carrier and/or excipient to a subject in need of such a treatment, wherein said H5N1 virus of a clade other than clade 1 is a H5N1 virus comprising:
a. a polynucleotide encoding a second H5 protein of influenza virus, wherein said second H5 protein encodes an amino acid sequence comprising:
i. the amino acids 113D, 126H, 145(−), 156R, 160F, 167T, and 181N, wherein the modification 145(−) means that amino acid position 145 of H5 is deleted, or
ii. the amino acids 87P, 145L, 172T, 201E, 206I, 208K, 254T, 341G and 421K, or
iii. the amino acids 145L, 172T, and 254V,
wherein the numbering of the amino acid positions of the second H5 protein of group (a), (b), and (c) refers to the amino acid position as exemplarily given in SEQ ID NO:8, or
iv. an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46; or
b. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137.

34. The method of claim 32, wherein said administration is a single-shot administration or a one dose administration.

35. A kit of parts, that comprises:
a. an H5 protein comprising a polypeptide sequence having at least 98% sequence identity with the polypeptide sequence of SEQ ID NO:1; and
b. a package leaflet indicating the use of such H5 protein according to a) for the treatment or prophylaxis of infections caused by H5N1 virus of a clade other than clade 1, wherein said H5N1 virus of a clade other than clade 1 is a H5N1 virus comprising:
i. a polynucleotide encoding a second H5 protein of influenza virus, wherein said second H5 protein encodes an amino acid sequence comprising:
1. the amino acids 113D, 126H, 145(−), 156R, 160F, 167T, and 181N, wherein the modification 145(−) means that amino acid position 145 of H5 is deleted, or
2. the amino acids 87P, 145L, 172T, 201E, 206I, 208K, 254T, 341G and 421K, or
3. the amino acids 145L, 172T, and 254V,
wherein the numbering of the amino acid positions of the second H5 protein of group (1), (2), and (3) refers to the amino acid position as exemplarily given in SEQ ID NO:8; or
4. an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46; or
ii. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137.

36. The kit according to claim 35, wherein such kit comprises at least one or more further antigens of poultry or mammalian pathogen.

37. The method according to claim 19, for use as a single-shot vaccine or in a one-dose vaccination.

38. A method of reducing the incidence of or severity of influenza infection, said influenza infection being with a H5N1 virus of a different clade, said method comprising the step of administering a composition comprising the H5 protein comprising a polypeptide sequence having at least 98% sequence identity with the polypeptide sequence of SEQ ID NO:1, wherein said H5N1 virus of a different clade is a H5N1 virus of a clade other than clade 1 comprising:
a. a polynucleotide encoding a second H5 protein of influenza virus, wherein said second H5 protein encodes an amino acid sequence comprising:
i. the amino acids 113D, 126H, 145(−), 156R, 160F, 167T, and 181N, wherein the modification 145(−) means that amino acid position 145 of H5 is deleted, or
ii. the amino acids 87P, 145L, 172T, 201E, 206I, 208K, 254T, 341G and 421K, or
iii. the amino acids 145L, 172T, and 254V, wherein the numbering of the amino acid positions of the second H5 protein of group (i), (ii), and (iii) refers to the amino acid position as exemplarily given in SEQ ID NO:8; or
iv. an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46, or
b. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137.

39. A method for reducing viral shedding in a subject comprising administering to said subject an H5 protein comprising a polypeptide sequence having at least 98% sequence identity with the polypeptide sequence of SEQ ID NO:1, wherein said H5 protein is to be administered to a subject infected with or at risk of a viral infection with H5N1 virus of a clade other than clade 1, and wherein said H5N1 virus of a clade other than clade 1 is a H5N1 virus comprising:
a. a polynucleotide encoding a second H5 protein of influenza virus, wherein said second H5 protein encodes an amino acid sequence comprising:
i. the amino acids 113D, 126H, 145(−), 156R, 160F, 167T, and 181N, wherein the modification 145(−) means that amino acid position 145 of H5 is deleted, or
ii. the amino acids 87P, 145L, 172T, 201E, 206I, 208K, 254T, 341G and 421K, or
iii. the amino acids 145L, 172T, and 254V, wherein the numbering of the amino acid positions of the second H5 protein of group (i), (ii), and (iii) refers to the amino acid position as exemplarily given in SEQ ID NO:8; or
iv. an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46; or
b. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137.

40. A method for reducing viral shedding in a subject infected with or at risk of a viral infection with H5N1 virus of a clade other than clade 1 comprising administering to said subject a medicament comprising an H5 protein comprising a polypeptide sequence having at least 98% sequence identity with the polypeptide sequence of SEQ ID NO:1, wherein said H5N1 virus of a clade other than clade 1 is a H5N1 virus comprising:
a. a polynucleotide encoding a second H5 protein of influenza virus, wherein said second H5 protein encodes an amino acid sequence comprising:
i. the amino acids 113D, 126H, 145(−), 156R, 160F, 167T, and 181N, wherein the modification 145(−) means that amino acid position 145 of H5 is deleted, or
ii. the amino acids 87P, 145L, 172T, 201E, 206I, 208K, 254T, 341G and 421K, or
iii. the amino acids 145L, 172T, and 254V,
wherein the numbering of the amino acid positions of the second H5 protein of group (i), (ii), and (iii) refers to the amino acid position as exemplarily given in SEQ ID NO:8; or
iv. an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137 or 9 to 46; or
b. a second H5 protein of influenza virus, wherein said second H5 protein comprises an amino acid sequence which is at least 95%, preferably at least 96%, more preferably at least 97%, still more preferably at least 98%, yet more preferably at least 99%, or in particular preferred 100% homolog with any one of the sequences as set forth in SEQ ID NOs: 62 to 137.
